SEQUENCE LISTING (86 sequences)

SEQ ID NO: 1 - DNA, 58 bp, Artificial Sequence
  OB1 Reverse adaptor
ctggggcacgggtaagaagcattctgtctctcttctaagaattcgagctcggtacccg

SEQ ID NO: 2 - DNA, 60 bp, Artificial Sequence
  OB2 AA-DTECT Adaptor
cgggtaccgagctcgaattcttagaagagagacagaatgcttcttacccgtgccccagaa

SEQ ID NO: 3 - DNA, 60 bp, Artificial Sequence
  OB3 AC-DTECT Adaptor
cgggtaccgagctcgaattcttagaagagagacagaatgcttcttacccgtgccccagac

SEQ ID NO: 4 - DNA, 60 bp, Artificial Sequence
  OB4 AG-DTECT Adaptor
cgggtaccgagctcgaattcttagaagagagacagaatgcttcttacccgtgccccagag

SEQ ID NO: 5 - DNA, 60 bp, Artificial Sequence
  OB5 AT-DTECT Adaptor
cgggtaccgagctcgaattcttagaagagagacagaatgcttcttacccgtgccccagat

SEQ ID NO: 6 - DNA, 60 bp, Artificial Sequence
  OB6 CA-DTECT Adaptor
cgggtaccgagctcgaattcttagaagagagacagaatgcttcttacccgtgccccagca

SEQ ID NO: 7 - DNA, 60 bp, Artificial Sequence
  OB7 CC-DTECT Adaptor
cgggtaccgagctcgaattcttagaagagagacagaatgcttcttacccgtgccccagcc

SEQ ID NO: 8 - DNA, 60 bp, Artificial Sequence
  OB8 CG-DTECT Adaptor
cgggtaccgagctcgaattcttagaagagagacagaatgcttcttacccgtgccccagcg

SEQ ID NO: 9 - DNA, 60 bp, Artificial Sequence
  OB9 CT-DTECT Adaptor
cgggtaccgagctcgaattcttagaagagagacagaatgcttcttacccgtgccccagct

SEQ ID NO: 10 - DNA, 60 bp, Artificial Sequence
  OB10 GA-DTECT Adaptor
cgggtaccgagctcgaattcttagaagagagacagaatgcttcttacccgtgccccagga

SEQ ID NO: 11 - DNA, 60 bp, Artificial Sequence
  OB11 GC-DTECT Adaptor
cgggtaccgagctcgaattcttagaagagagacagaatgcttcttacccgtgccccaggc

SEQ ID NO: 12 - DNA, 60 bp, Artificial Sequence
  OB12 GG-DTECT Adaptor
cgggtaccgagctcgaattcttagaagagagacagaatgcttcttacccgtgccccaggg

SEQ ID NO: 13 - DNA, 60 bp, Artificial Sequence
  OB13 GT-DTECT Adaptor
cgggtaccgagctcgaattcttagaagagagacagaatgcttcttacccgtgccccaggt

SEQ ID NO: 14 - DNA, 60 bp, Artificial Sequence
  OB14 TA-DTECT Adaptor
cgggtaccgagctcgaattcttagaagagagacagaatgcttcttacccgtgccccagta

SEQ ID NO: 15 - DNA, 60 bp, Artificial Sequence
  OB15 TC-DTECT Adaptor
cgggtaccgagctcgaattcttagaagagagacagaatgcttcttacccgtgccccagtc

SEQ ID NO: 16 - DNA, 60 bp, Artificial Sequence
  OB16 TG-DTECT Adaptor
cgggtaccgagctcgaattcttagaagagagacagaatgcttcttacccgtgccccagtg

SEQ ID NO: 17 - DNA, 60 bp, Artificial Sequence
  OB17 TT-DTECT Adaptor
cgggtaccgagctcgaattcttagaagagagacagaatgcttcttacccgtgccccagtt

SEQ ID NO: 18 - DNA, 25 bp, Artificial Sequence
  OB18 PB1072
gcaattcctcacgagacccgtcctg

SEQ ID NO: 19 - DNA, 27 bp, Artificial Sequence
  OB19 PB1073
cgggtaccgagctcgaattcttagaag

SEQ ID NO: 20 - DNA, 27 bp, Artificial Sequence
  OB20 Fwd del69-70
tttttcttgttttattgccactagtct

SEQ ID NO: 21 - DNA, 27 bp, Artificial Sequence
  OB21 Rev del69-70
caacttttgttgtttttgtggtaataa

SEQ ID NO: 22 - DNA, 60 bp, Artificial Sequence
  OB22 AcuI#1 del69-70
gcaattcctcacgagacccgtcctgtttccaatgttacttctgaagggttccatgctata

SEQ ID NO: 23 - DNA, 60 bp, Artificial Sequence
  OB23 AcuI#2 del69-70
gcaattcctcacgagacccgtcctgttttccaatgttactctgaagtggttccatgctat

SEQ ID NO: 24 - DNA, 60 bp, Artificial Sequence
  OB24 AcuI#3 del69-70
gcaattcctcacgagacccgtcctgtcaaacctcttagtactgaagccattggtcccaga

SEQ ID NO: 25 - DNA, 60 bp, Artificial Sequence
  OB25 AcuI#4 del69-70
gcaattcctcacgagacccgtcctgatcaaacctcttagtctgaagaccattggtcccag

SEQ ID NO: 26 - DNA, 27 bp, Artificial Sequence
  OB26 Fwd RBD
actgtgttgctgattattctgtcctat

SEQ ID NO: 27 - DNA, 27 bp, Artificial Sequence
  OB27 Rev RBD
gtaatgtcaagaatctcaagtgtctgt

SEQ ID NO: 28 - DNA, 60 bp, Artificial Sequence
  OB28 AcuI#1 K417N
gcaattcctcacgagacccgtcctgagacaaatcgctccactgaaggggcaaactggaaa

SEQ ID NO: 29 - DNA, 60 bp, Artificial Sequence
  OB29 AcuI#2 K417N AcuI#1 K417T
gcaattcctcacgagacccgtcctgcagacaaatcgctccctgaagagggcaaactggaa

SEQ ID NO: 30 - DNA, 60 bp, Artificial Sequence
  OB30 AcuI#2 K417T
gcaattcctcacgagacccgtcctgtcagacaaatcgctcctgaagcagggcaaactgga

SEQ ID NO: 31 - DNA, 60 bp, Artificial Sequence
  OB31 AcuI#3 K417T
gcaattcctcacgagacccgtcctgctggtaatttataatctgaagtataatcagcaatc

SEQ ID NO: 32 - DNA, 60 bp, Artificial Sequence
  OB32 AcuI#4 K417T AcuI#3 K417N
gcaattcctcacgagacccgtcctgtctggtaatttataactgaagttataatcagcaat

SEQ ID NO: 33 - DNA, 60 bp, Artificial Sequence
  OB33 AcuI#4 K417N
gcaattcctcacgagacccgtcctgatctggtaatttatactgaagattataatcagcaa

SEQ ID NO: 34 - DNA, 60 bp, Artificial Sequence
  OB34 AcuI#1 S477N
gcaattcctcacgagacccgtcctgtatttcaactgaaatctgaagctatcaggccggta

SEQ ID NO: 35 - DNA, 60 bp, Artificial Sequence
  OB35 AcuI#2 S477N
gcaattcctcacgagacccgtcctgatatttcaactgaaactgaagtctatcaggccggt

SEQ ID NO: 36 - DNA, 60 bp, Artificial Sequence
  OB36 AcuI#3 S477N
gcaattcctcacgagacccgtcctgtaaaaccttcaacacctgaagcattacaaggtgtg

SEQ ID NO: 37 - DNA, 60 bp, Artificial Sequence
  OB37 AcuI#4 S477N
gcaattcctcacgagacccgtcctgttaaaaccttcaacactgaagccattacaaggtgt

SEQ ID NO: 38 - DNA, 60 bp, Artificial Sequence
  OB38 AcuI#1 E484K
gcaattcctcacgagacccgtcctgaggccggtagcacacctgaagcttgtaatggtgtt

SEQ ID NO: 39 - DNA, 60 bp, Artificial Sequence
  OB39 AcuI#2 E484K
gcaattcctcacgagacccgtcctgcaggccggtagcacactgaagccttgtaatggtgt

SEQ ID NO: 40 - DNA, 60 bp, Artificial Sequence
  OB40 AcuI#3 E484K
gcaattcctcacgagacccgtcctgttgtaaaggaaagtactgaagacaattaaaacctt

SEQ ID NO: 41 - DNA, 60 bp, Artificial Sequence
  OB41 AcuI#4 E484K
gcaattcctcacgagacccgtcctgattgtaaaggaaagtctgaagaacaattaaaacct

SEQ ID NO: 42 - DNA, 60 bp, Artificial Sequence
  OB42 AcuI#1 Q498R
gcaattcctcacgagacccgtcctgttactttcctttacactgaagatcatatggtttcc

SEQ ID NO: 43 - DNA, 60 bp, Artificial Sequence
  OB43 AcuI#2 Q498R
gcaattcctcacgagacccgtcctggttactttcctttacctgaagaatcatatggtttc

SEQ ID NO: 44 - DNA, 60 bp, Artificial Sequence
  OB44 AcuI#3 Q498R
gcaattcctcacgagacccgtcctgatggttggtaaccaactgaagcaccattagtgggt

SEQ ID NO: 45 - DNA, 60 bp, Artificial Sequence
  OB45 AcuI#4 Q498R
gcaattcctcacgagacccgtcctgtatggttggtaaccactgaagacaccattagtggg

SEQ ID NO: 46 - DNA, 60 bp, Artificial Sequence
  OB46 AcuI#1 N501Y
gcaattcctcacgagacccgtcctgctttacaatcatatgctgaaggtttccaacccact

SEQ ID NO: 47 - DNA, 60 bp, Artificial Sequence
  OB47 AcuI#2 N501Y
gcaattcctcacgagacccgtcctgcctttacaatcatatctgaagggtttccaacccac

SEQ ID NO: 48 - DNA, 60 bp, Artificial Sequence
  AcuI#3 N501Y
gcaattcctcacgagacccgtcctgtactctgtatggttgctgaaggtaaccaacaccat

SEQ ID NO: 49 - DNA, 60 bp, Artificial Sequence
  OB49 AcuI#4 N501Y
gcaattcctcacgagacccgtcctgctactctgtatggttctgaagggtaaccaacacca

SEQ ID NO: 50 - DNA, 1151 bp, Artificial Sequence
  SARS-CoV-2 WT (encodes 2 regions of the reference S protein)
atcaagctttttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttgactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacggatccatc

SEQ ID NO: 51 - DNA, 1145 bp, Artificial Sequence
  SARS-CoV-2 del69-70 K417T S477N Q498R (encodes 2 regions of the S protein with 4 variants)
atcaagctttttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatatctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttgactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaacgattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtaacacaccttgtaatggtgttgaaggttttaattgttactttcctttacaatcatatggtttccgacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacggatccatc

SEQ ID NO: 52 - DNA, 703 bp, Artificial Sequence
  SARS-CoV-2 K417N, E484K and N501Y(encodes 2 regions of the S protein with 3 variants)
atcaagcttactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaatattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttaaaggttttaattgttactttcctttacaatcatatggtttccaacccacttatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacggatccatc

SEQ ID NO: 53 - DNA, 25 bp, Artificial Sequence
  a non-complementary handle sequence of 25 nt
gcaattcctcacgagacccgtcctg

SEQ ID NO: 54 - DNA, 35 bp, Artificial Sequence
  AcuI tagging primer
  misc_feature (1)..(15): n is a, c, g, or t
  misc_feature (22)..(35): n is a, c, g, or t
nnnnnnnnnnnnnnnctgaagnnnnnnnnnnnnnn

SEQ ID NO: 55 - DNA, 25 bp, Artificial Sequence
  non-complementary handle sequence of 25 nt
gcaattcctcacgagacccgtcctg

SEQ ID NO: 56 - DNA, 35 bp, Artificial Sequence; identical to SEQ ID NO: 54
  Acu tagging primer
  misc_feature (1)..(15): n is a, c, g, or t
  misc_feature (22)..(35): n is a, c, g, or t

SEQ ID NO: 57 - DNA, 40 bp, Artificial Sequence
  a non-complementary handle sequence
ctgcacctcatggtcatgttatggttgagctggtagcaga

SEQ ID NO: 58 - DNA, 39 bp, Artificial Sequence
  a non-complementary handle sequence for ORF1a detection
tggctactaccgaagagctaccagacgaattcgtggtgg

SEQ ID NO: 59 - DNA, 30 bp, Artificial Sequence
  .OB196
agcctgtggttcctgaagatcgcgtccgat

SEQ ID NO: 60 - DNA, 30 bp, Artificial Sequence
  OB197
atcggacgcgatcttcaggaaccacaggct

SEQ ID NO: 61 - DNA, 30 bp, Artificial Sequence
  control competitor
agcctgtggttcaaagtcatcgcgtccgat

SEQ ID NO: 62 - DNA, 30 bp, Artificial Sequence
  control competitor
atcggacgcgatgactttgaaccacaggct

SEQ ID NO: 63 - DNA, 75 bp, Artificial Sequence
  OB128 AcuI LAMP-1a E484K
ctgcacctcatggtcatgttatggttgagctggtagcagattgtaaaggaaagtactgaagacaattaaaacctt

SEQ ID NO: 64 - DNA, 20 bp, Artificial Sequence
  OB129 F3-1a
ctgcacctcatggtcatgtt

SEQ ID NO: 65 - DNA, 42 bp, Artificial Sequence
  OB130 FIP-1a
gagggacaaggacaccaagtgtatggttgagctggtagcaga

SEQ ID NO: 66 - DNA, 19 bp, Artificial Sequence
  OB131 B3-1a
agctcgtcgcctaagtcaa

SEQ ID NO: 67 - DNA, 39 bp, Artificial Sequence
  OB132 BIP-1a
ccagtggcttaccgcaaggttttagatcggcgccgtaac

SEQ ID NO: 68 - DNA, 25 bp, Artificial Sequence
  OB133 LB-1a
ttcgtaagaacggtaataaaggagc

SEQ ID NO: 69 - DNA, 74 bp, Artificial Sequence
  OB134 AcuI LAMP-N E484K
tggctactaccgaagagctaccagacgaattcgtggtggttgtaaaggaaagtactgaagacaattaaaacctt

SEQ ID NO: 70 - DNA, 19 bp, Artificial Sequence
  OB135 F3-N
tggctactaccgaagagct

SEQ ID NO: 71 - DNA, 41 bp, Artificial Sequence
  OB136 FIP-N
tctggcccagttcctaggtagtccagacgaattcgtggtgg

SEQ ID NO: 72 - DNA, 20 bp, Artificial Sequence
  OB137 B3-N
tgcagcattgttagcaggat

SEQ ID NO: 73 - DNA, 40 bp, Artificial Sequence
  OB138 BIP-N
agacggcatcatatgggttgcacgggtgccaatgtgatct

SEQ ID NO: 74 - DNA, 21 bp, Artificial Sequence
  OB139 LB-N
actgagggagccttgaataca

SEQ ID NO: 75 - DNA, 140 bp, Artificial Sequence
  OB140 DTECT_LAMP_1a_constant
acacttggtgtccttgtccctcatgtgggcgaaataccagtggcttaccgcaaggttcttcttcgtaagaacggtaataaaggagctggtggccatagttacggcgccgatctaaagtcatttgacttaggcgacgagct

SEQ ID NO: 76 - DNA, 142 bp, Artificial Sequence
  OB141 DTECT_LAMP_1a_TA
agctcgtcgcctaagtcaaatgactttagatcggcgccgtaactatggccaccagctcctttattaccgttcttacgaagaagaaccttgcggtaagccactggtatttcgcccacatgagggacaaggacaccaagtgtta

SEQ ID NO: 77 - DNA, 142 bp, Artificial Sequence
  OB142 DTECT_LAMP_1a_TG
agctcgtcgcctaagtcaaatgactttagatcggcgccgtaactatggccaccagctcctttattaccgttcttacgaagaagaaccttgcggtaagccactggtatttcgcccacatgagggacaaggacaccaagtgttg

SEQ ID NO: 78 - DNA, 137 bp, Artificial Sequence
  OB143 DTECT_LAMP_N_constant
actacctaggaactgggccagaagctggacttccctatggtgctaacaaagacggcatcatatgggttgcaactgagggagccttgaatacaccaaaagatcacattggcacccgcaatcctgctaacaatgctgca

SEQ ID NO: 79 - DNA, 139 bp, Artificial Sequence
  OB144 DTECT_LAMP_N_TA
tgcagcattgttagcaggattgcgggtgccaatgtgatcttttggtgtattcaaggctccctcagttgcaacccatatgatgccgtctttgttagcaccatagggaagtccagcttctggcccagttcctaggtagtta

SEQ ID NO: 80 - DNA, 139 bp, Artificial Sequence
  OB145 DTECT_LAMP_N_TG
tgcagcattgttagcaggattgcgggtgccaatgtgatcttttggtgtattcaaggctccctcagttgcaacccatatgatgccgtctttgttagcaccatagggaagtccagcttctggcccagttcctaggtagttg

SEQ ID NO: 81 - DNA, 30 bp, Artificial Sequence; identical to SEQ ID NO: 59
  OB196 Competitor AcuI for

SEQ ID NO: 82 - DNA, 30 bp, Artificial Sequence; identical to SEQ ID NO: 60
  OB197 Competitor AcuI rev

SEQ ID NO: 83 - DNA, 30 bp, Artificial Sequence; identical to SEQ ID NO: 61
  OB198 Competitor CTL for

SEQ ID NO: 84 - DNA, 30 bp, Artificial Sequence; identical to SEQ ID NO: 62
  OB199 Competitor CTL rev

SEQ ID NO: 85 - DNA, 20 bp, Artificial Sequence
  3′ end of OB1
gaattcgagctcggtacccg

SEQ ID NO: 86 - DNA, 120 bp, Artificial Sequence
  Figure 14a
gcaattcctcacgagacccgtcctgttgtaaaggaaagtactgaagacaattaaaacctttactggggcacgggtaagaagcattctgtctctcttctaagaattcgagctcggtacccg

(19) United States
(12) Patent Application Publication
(10) Pub. No.: US 2024/0279728 A1
(43) Pub. Date: Aug. 22, 2024

(54) DETECTING A DINUCLEOTIDE SEQUENCE IN A TARGET POLYNUCLEOTIDE

(71) Applicant: UTI Limited Partnership, Calgary (CA)

(72) Inventors: Pierre BILLON, Calgary (CA); Lou Anne Ghyslaine BAUDRIER, Calgary (CA); Orlena Aviela Camille BENAMOZIG, Rehovot (IL)

(21) Appl. No.: 18/568,668

(22) PCT Filed: Jun. 8, 2022
(86) PCT No.: PCT/CA2022/050914

§ 371 Date: Dec. 8, 2023

Related U.S. Application Data
(60) Provisional application No. 63/209,619, filed on Jun. 11, 2021.

(51) Int. Cl. C12Q 1/6853

(52) U.S. Cl. CPC C12Q 1/6853

(57) ABSTRACT

The present disclosure relates to improvements in the Dinucleotide signaTurE CapTure (DTECT) method. The improved detection of a dinucleotide sequence in a target polynucleotide generally involves the steps of Acu1 digestion, heat inactivation and ligation to unique adaptors containing overhangs of two bases complementary to the dinucleotide signature.

### CROSS REFERENCE TO RELATED APPLICATION

This application claims priority to United States Provisional Patent Application U.S. 63/209,619, filed on 11 Jun. 2021, the entire contents of which is hereby incorporated by reference.

### FIELD

The present disclosure relates generally to a detecting a dinucleotide sequence in a target polynucleotide.

### BACKGROUND

Sequencing technologies, such as Sanger or Next-Generation Sequencing, are the most common methods to detect genomic sequences and variants of interest. Despite their high accuracy, these technologies remain time-consuming and expensive. There is currently no rapid and cost-efficient method that can be efficiently conducted using all-in-one reactions to detect desired genetic signatures.

Identifying variations in DNA sequences is a routine task in basic research for genetic testing, clinical diagnostic, or forensic purposes. Over the past decades, several technologies utilizing Sanger or Next-Generation Sequencing (NGS) platforms have been developed to facilitate the sequencing of DNA molecules, enabling the determination of DNA sequences and the identification of variants. However, although these technologies are easily accessible, as companies offer genomic platforms and sample processing, they remain time-consuming (several days to weeks) and expensive (from several dollars to thousand dollars) for routine laboratory experiments. Moreover, it requires the processing of samples by third parties, which can cause errors and contaminations during sample manipulation. The past two decades have also witnessed the development of an accelerated number of new techniques using variant-specific primers or probes. However, these techniques are not robust because the efficacy and specificity of the detection are strongly dependent on the sequence and mutation. In addition, these approaches lack specificities as they rely on weak and transient nucleic acid interactions to distinguish between genetic variants, which often differ from the reference by only one nucleotide.

The severe acute respiratory syndrome coronavirus 2 (SARS-CoV-2), the etiologic agent of the coronavirus disease 2019 (COVID-19), has spread rapidly becoming a global pandemic¹⁻³. Despite the implementation of government-imposed mitigation measures, nationwide lockdowns, and worldwide travel bans, COVID-19 has caused more than 3 million deaths and is responsible for a high incidence of long-lasting COVID-19 symptoms⁴⁻⁶. Surveillance of circulating viruses revealed the emergence of variants carrying multiple concerning mutations^{7,8} capable of partially evading immune response, enhance virus transmission, and disease severity⁹⁻²².

The detection of SARS-CoV-2 nucleic acids in patient samples employs RT-PCR. However, RT-PCR does not identify variants²³.

Genomic surveillance strategies for SARS-CoV-2 variants are primarily limited to the sequencing of viral nucleic acids isolated from infected humans^{24,25}. Rapid detection methods to detect the presence of variants utilize mutation-specific primers and probes²⁶⁻²⁸. However, these approaches inherently exhibit low specificity because they rely on weak nucleic acid interactions to discriminate variants with only a single nucleotide difference to the reference.

The sequencing of SARS-CoV-2 genomes plays a fundamental role in the discovery of new emerging variants^{24,25}. However, sequencing cannot substitute for the development of rapid routine tests for circulating variants. Indeed, sequencing requires sophisticated technologies^{29,30}, has a high error rate requiring the deployment of complex bioinformatic pipelines³¹, is expensive, slow (several days), and susceptible to contaminations³². Therefore, the implementation of reliable, rapid, and cost-effective diagnostic tools into standard diagnostic platforms is needed to contain the propagation of the variants.

Despite the importance of detecting genetic sequences and associated mutations, there are still no all-in-one assays available for rapid detection of genetic signatures for routine laboratory experiments.

### SUMMARY

As described herein, there is provided:
1. A method of detecting a dinucleotide sequence in a target polynucleotide containing sample from a subject, the method comprising:
(a) contacting the target polynucleotide containing sample with (i) at least one Acu1 tagging primer, and (ii) a at least one reverse Acu1 primer, under condition to generate an Acu1-tagged amplicon;
(b) subjecting the Acu1-tagged amplicon to a digestion with Acu1 to generate a digestion reaction mixture comprising a digested Acu1-tagged amplicon;
(c) subjecting the digestion reaction mixture to a heat inactivation step to generate a heat inactivation reaction mixture;
(d) contacting the heat inactivation reaction mixture with a one or more adaptors under conditions to ligate said one or more adaptors to the digested Acu1-tagged amplicon, to generate a ligation product; and
(e) detecting said ligated product.
2. The method of item 1, wherein said Acu1 tagging primer comprises an Acu1 motif polynucleotide (5′-CTGAAG-3′) positioned 14 bases from the 3′ end of the Acu1 tagging primer.
3. The method of item 1 or 2, wherein the Acu1 tagging primer comprises a detection handle positioned at the 5′ end of the Acu1 tagging primer.
4. The method of item 3, wherein the detection handle comprises or consists of the sequence 5′-GCAATTCCTCACGAGACCCGTCCTG-3′ (SEQ ID NO: 53).
5. The method of any one of items 1 to 4, wherein subjecting the Acu1-tagged amplicon to a digestion with Acu1 to generate a digestion reaction mixture comprising a digested Acu1-tagged amplicon is for about 1 minute at about 37° C.
6. The method of any one of items 1 to 5, wherein said heat inactivation steps comprises heating for about 1 minute to about 10 minutes at about 65° C.
7. The method of any one of items 1 to 6, wherein step (d) comprises a first step for about 10 minutes at about 65° C., a heating step for about 10 minutes.
8. The method of any one of items 1 to 7, wherein said detecting is quantitative, semi-quantitative, analytical, or visual.
9. The method of any one of items 1 to 8, wherein the sample is from a eukaryote, a prokaryote, or a virus.
10. The method of any one of items 1 to 8, wherein the subject is a mammal, a plant, a bacterium, a fungus, a protest, or a virus.
11. The method of any one of items 1 to 10, wherein the sample is isolated from a cell, a cell pellet, a cell extract, a tissue, a biopsy, or biological fluid, obtained from the subject.
12. The method of any one of items 1 to 11, wherein the target polynucleotide is a severe acute respiratory syndrome coronavirus 2 (SARS-CoV-2) polynucleotide sample.
13. The method of any one of items 1 to 11, wherein the target polynucleotide is the PIK3R1 gene, a DNA repair gene, or PCNA.
14. The method of any one of items 1 to 13, wherein the dinucleotide is a mutation, or a reference sequence.
15. The method of item 14, wherein the mutation is a transition, transversion, insertion, or deletion.
16. The method of any one of items 1 to 15, wherein the sample is from a cancer sample.
17. The method of any one of items 1 to 16, wherein the sample is from a nasal swab, a sample from an oropharyngeal swab, a sputum sample, a lower respiratory tract aspirate, a bronchoalveolar lavage, a nasopharyngeal wash/aspirate or a nasal aspirate.
18. The method of any one of items 1 to 17, wherein the subject is a human.
19. A method of detecting a dinucleotide sequence in a target polynucleotide containing sample from a subject, the method comprising:
(a) contacting the target polynucleotide containing sample with (i) at least one Acu1 tagging primer, and (ii) a at least one reverse Acu1 primer, under condition to generate an Acu1-tagged amplicon;
(b) subjecting the Acu1-tagged amplicon to (i) a digestion with Acu1 to generate a digestion reaction mixture comprising a digested Acu1-tagged amplicon, and (ii) a one or more variant adaptors under condition to ligate said one or more adaptors to the digested Acu1-tagged amplicon, to generate a ligation product;
(c) subjecting the digestion reaction mixture to a heat inactivation step to generate a heat inactivation reaction mixture;
(d) detecting said ligated product.
20. The method of item 19, wherein said Acu1 tagging primer comprises an Acu1 motif polynucleotide (5′-CTGAAG-3′) positioned 14 bases from the 3′ end of the Acu1 tagging primer.
21. The method of item 19 or 20, wherein the Acu1 tagging primer comprises a detection handle positioned at the 5′ end of the Acu1 tagging primer.
22. The method of item 21, wherein the detection handle comprises or consists of the sequence 5′-GCAATTCCTCACGAGACCCGTCCTG-3′ (SEQ ID NO: 53).
23. The method of any one of items 19 to 22, wherein the reaction conditions of step (b) are carried out for about 1 minute to 1 hour at room temperature.
24. The method of any one of items 19 to 23, wherein the reaction conditions of step (b) are carried out for about 1 minute to 1 hour at room temperature.
25. A method of detecting a dinucleotide sequence in a target polynucleotide containing sample from a subject, the method comprising:
(a) contacting the target polynucleotide containing sample with (i) at least one Acu1 tagging primer, and (ii) a at least one reverse Acu1 primer, under condition to generate an Acu1-tagged amplicon;
(b) subjecting the Acu1-tagged amplicon to (i) a digestion with Acu1 to generate a digestion reaction mixture comprising a digested Acu1-tagged amplicon, and (ii) a one or more variant adaptors under condition to ligate said one or more adaptors to the digested Acu1-tagged amplicon, to generate a ligate product;
(c) subjecting the digestion reaction mixture to a heat inactivation step to generate a heat inactivation reaction mixture;
(d) detecting said ligated product.
26. The method of item 25, wherein said Acu1 tagging primer comprises an Acu1 motif polynucleotide (5′-CTGAAG-3′) positioned 14 bases from the 3′ end of the Acu1 tagging primer.
27. The method of item 25 or 26, wherein the Acu1 tagging primer comprises a detection handle positioned at the 5′ end of the Acu1 tagging primer.
28. The method of item 27, wherein the detection handle comprises or consists of the sequence 5′-GCAATTCCTCACGAGACCCGTCCTG-3′ (SEQ ID NO: 53).
29. The method of any one of items 25 to 28, wherein the reaction conditions of step (b) are carried out for about 1 minute to 1 hour at room temperature.
30. The method of item 25 or 29, wherein the reaction conditions of step (b) are carried out for about 1 minute to 1 hour at room temperature.
31. The method of any one of items 19 to 30, wherein subjecting the Acu1-tagged amplicon to a digestion with Acu1 to generate a digestion reaction mixture comprising a digested Acu1-tagged amplicon is for about 1 minute at about 37° C.
32. The method of any one of items 19 to 31, wherein said heat inactivation steps comprises heating for about 1 minute to about 10 minutes at about 65° C.
33. The method of any one of items 19 to 32, wherein step (d) comprises a first step for about 10 minutes at about 65° C., a heating step for about 10 minutes.
34. The method of any one of items 19 to 33, wherein said detecting is quantitative, semi-quantitative, analytical, or visual.
35. The method of any one of items 19 to 34, wherein the sample is from a eukaryote, a prokaryote, or a virus.
36. The method of any one of items 19 to 34, wherein the subject is a mammal, a plant, a bacterium, a fungus, a protest, or a virus.
37. The method of any one of items 19 to 36, wherein the sample is isolated from a cell, a cell pellet, a cell extract, a tissue, a biopsy, or biological fluid, obtained from the subject.
38. The method of any one of items 19 to 37, wherein the target polynucleotide is a severe acute respiratory syndrome coronavirus 2 (SARS-CoV-2) polynucleotide sample.
39. The method of any one of items 19 to 37, wherein the target polynucleotide is the PIK3R1 gene, a DNA repair gene, or PCNA.
40. The method of any one of items 19 to 39, wherein the dinucleotide is a mutation, or a reference sequence.
41. The method of item 40, wherein the mutation is a transition, transversion, insertion, or deletion.
42. The method of any one of items 19 to 41, wherein the sample is from a cancer sample.
43. The method of any one of items 19 to 42, wherein the sample is from a nasal swab, a sample from an oropharyngeal swab, a sputum sample, a lower respiratory tract aspirate, a bronchoalveolar lavage, a nasopharyngeal wash/aspirate or a nasal aspirate.
44. The method of any one of items 19 to 43, wherein the subject is a human.
45. Kit comprising a 2× DTECT reaction for single-step capture, a library of 16 adaptors, a container, master mixes for analytical, quantitative or visual detection, and optionally instructions for the use thereof, said adaptor comprising a double-stranded DNA formed by the annealing of two complementary oligonucleotides; one of the two strand contains a 3′ dinucleotide overhang that is used to capture the complementary variant signature.
46. A kit comprising one or more isolated polynucleotide selected from:

| | | | |
|---|---|---|---|
| OB1 | Reverse adaptor | CTGGGGCACGGGTAAGAAGCATTCTGTCTCTCTTCTAAgaattcgagctcggtacccg | 1 |
| OB2 | AA-DTECT Adaptor | cgggtaccgagctcgaattcTTAGAAGAGAGACAGAATGCTTCTTACCCGTGCCCCAGAA | 2 |
| OB3 | AC-DTECT Adaptor | cgggtaccgagctcgaattcTTAGAAGAGAGACAGAATGCTTCTTACCCGTGCCCCAGAC | 3 |
| OB4 | AG-DTECT Adaptor | cgggtaccgagctcgaattcTTAGAAGAGAGACAGAATGCTTCTTACCCGTGCCCCAGAG | 4 |
| OB5 | AT-DTECT Adaptor | cgggtaccgagctcgaattcTTAGAAGAGAGACAGAATGCTTCTTACCCGTGCCCCAGAT | 5 |
| OB6 | CA-DTECT Adaptor | cgggtaccgagctcgaattcTTAGAAGAGAGACAGAATGCTTCTTACCCGTGCCCCAGCA | 6 |
| OB7 | CC-DTECT Adaptor | cgggtaccgagctcgaattcTTAGAAGAGAGACAGAATGCTTCTTACCCGTGCCCCAGCC | 7 |
| OB8 | CG-DTECT Adaptor | cgggtaccgagctcgaattcTTAGAAGAGAGACAGAATGCTTCTTACCCGTGCCCCAGCG | 8 |
| OB9 | CT-DTECT Adaptor | cgggtaccgagctcgaattcTTAGAAGAGAGACAGAATGCTTCTTACCCGTGCCCCAGCT | 9 |
| OB10 | GA-DTECT Adaptor | cgggtaccgagctcgaattcTTAGAAGAGAGACAGAATGCTTCTTACCCGTGCCCCAGGA | 10 |
| OB11 | GC-DTECT Adaptor | cgggtaccgagctcgaattcTTAGAAGAGAGACAGAATGCTTCTTACCCGTGCCCCAGGC | 11 |
| OB12 | GG-DTECT Adaptor | cgggtaccgagctcgaattcTTAGAAGAGAGACAGAATGCTTCTTACCCGTGCCCCAGGG | 12 |
| OB13 | GT-DTECT Adaptor | cgggtaccgagctcgaattcTTAGAAGAGAGACAGAATGCTTCTTACCCGTGCCCCAGGT | 13 |
| OB14 | TA-DTECT Adaptor | cgggtaccgagctcgaattcTTAGAAGAGAGACAGAATGCTTCTTACCCGTGCCCCAGTA | 14 |
| OB15 | TC-DTECT Adaptor | cgggtaccgagctcgaattcTTAGAAGAGAGACAGAATGCTTCTTACCCGTGCCCCAGTC | 15 |
| OB16 | TG-DTECT Adaptor | cgggtaccgagctcgaattcTTAGAAGAGAGACAGAATGCTTCTTACCCGTGCCCCAGTG | 16 |
| or | | | |
| OB17 | TT-DTECT Adaptor | cgggtaccgagctcgaattcTTAGAAGAGAGACAGAATGCTTCTTACCCGTGCCCCAGTT | 17 |

and a container, and optionally instructions for use thereof.
47. A method of detecting a dinucleotide sequence in a target sequence of an infectious agent polynucleotide sample, the method comprising:
(a) contacting the target polynucleotide containing sample with (i) at least one Acu1 tagging primer, and (ii) a at least one reverse Acu1 primer, under condition to generate an Acu1-tagged amplicon;
(b) subjecting the Acu1-tagged amplicon to a digestion with Acu1 to generate a digestion reaction mixture comprising a digested Acu1-tagged amplicon;
(c) subjecting the digestion reaction mixture to a heat inactivation step to generate a heat inactivation reaction mixture;
(d) contacting the heat inactivation reaction mixture with a one or more variant adaptors under conditions to ligate said one or more adaptors to the digested Acu1-tagged amplicon, to generate a ligation product; and (e) detecting said ligated product.
48. The method of item 47, wherein said Acu1 tagging primer comprises an Acu1 motif polynucleotide (5′-CTGAAG-3′) positioned 14 bases from the 3′ end of the Acu1 tagging primer.
49. The method of item 46 or 47, wherein the Acu1 tagging primer comprises a detection handle positioned at the 5′ end of the Acu1 tagging primer.
50. The method of item 49, wherein the detection handle comprises or consists of the sequence 5′-GCAATTCCTCACGAGACCCGTCCTG-3′ (SEQ ID NO: 53).
51. The method of any one of items 47 to 50, wherein the infectious agent is SARS CoV2, Deltavirus, Adenoviridae, Anelloviridae, Arenaviridae, Astroviridae, Caliciviridae, Coronaviridae, Filoviridae, Flaviviridae, Hantaviridae, Hepadnaviridae, Hepeviridae, Herpesviridae, Nairoviridae, Orthomyxoviridae, Papillomaviridae, Paramyxoviridae, Parvoviridae, Peribunyaviridae, Phenuviridae, Picornaviridae, Pneumoviridae, Polyomaviridae, Poxviridae, Reoviridae, Retroviridae, Rhabdoviridae, Smacoviridae, or Togaviridae.
52. The method of any one of items 47 to 51, wherein subjecting the Acu1-tagged amplicon to a digestion with Acu1 to generate a digestion reaction mixture comprising a digested Acu1-tagged amplicon is for about 1 minute at about 37° C.
53. The method of any one of items 47 to 52, wherein said heat inactivation steps comprises heating for about 1 minute to about 10 minutes at about 65° C.
54. The method of any one of items 47 to 52, wherein step (d) comprises a first step for about 10 minutes at about 65° C., a heating step for about 10 minutes.
55. The method of any one of items 47 to 54, wherein the conditions to ligate said one or more adaptors comprises using T4 ligase or T3 ligase.
56. A method of detecting a dinucleotide sequence in a target sequence of an infectious agent polynucleotide sample, the method comprising:
(a) contacting the target polynucleotide containing sample with (i) at least one Acu1 tagging primer, and (ii) a at least one reverse Acu1 primer, under condition to generate an Acu1-tagged amplicon;
(b) subjecting the Acu1-tagged amplicon to (i) a digestion with Acu1 to generate a digestion reaction mixture comprising a digested Acu1-tagged amplicon, and (ii) a one or more variant adaptors under condition to ligate said one or more adaptors to the digested Acu1-tagged amplicon, to generate a ligate product;
(c) subjecting the digestion reaction mixture to a heat inactivation step to generate a heat inactivation reaction mixture;
(d) detecting said ligated product.
57. The method of item 56, wherein said Acu1 tagging primer comprises an Acu1 motif polynucleotide (5′-CTGAAG-3′) positioned 14 bases from the 3′ end of the Acu1 tagging primer.
58. The method of item 56 or 57, wherein the Acu1 tagging primer comprises a detection handle positioned at the 5′ end of the Acu1 tagging primer.
59. The method of item 58, wherein the detection handle comprises or consists of the sequence 5′-GCAATTCCTCACGAGACCCGTCCTG-3′ (SEQ ID NO: 53).
60. The method of any one of items 56 to 59, wherein the infectious agent is SARS CoV2, Deltavirus, Adenoviridae, Anelloviridae, Arenaviridae, Astroviridae, Caliciviridae, Coronaviridae, Filoviridae, Flaviviridae, Hantaviridae, Hepadnaviridae, Hepeviridae, Herpesviridae, Nairoviridae, Orthomyxoviridae, Papillomaviridae, Paramyxoviridae, Parvoviridae, Peribunyaviridae, Phenuviridae, Picornaviridae, Pneumoviridae, Polyomaviridae, Poxviridae, Reoviridae, Retroviridae, Rhabdoviridae, Smacoviridae, or Togaviridae.
61. The method of any one of items 56 to 60, wherein the reaction conditions of step (b) are carried out for about 10 minutes at room temperature.
62. The method of any one of items 56 to 61, wherein the heat inactivation step comprises heating for about 1 minute at about 65° C.
63. A method of detecting a dinucleotide sequence in a target sequence of an infectious agent polynucleotide sample, the method comprising:
(a) contacting the target polynucleotide containing sample with (i) at least one Acu1 tagging primer, and (ii) a at least one reverse Acu1 primer, under condition to generate an Acu1-tagged amplicon;
(b) subjecting the Acu1-tagged amplicon to (i) a digestion with Acu1 to generate a digestion reaction mixture comprising a digested Acu1-tagged amplicon, and (ii) a one or more variant adaptors under condition to ligate said one or more adaptors to the digested Acu1-tagged amplicon, to generate a ligate product;
(c) subjecting the digestion reaction mixture to a heat inactivation step to generate a heat inactivation reaction mixture;
(d) detecting said ligated product.
64. The method of item 63, wherein said Acu1 tagging primer comprises an Acu1 motif polynucleotide (5′-CTGAAG-3′) positioned 14 bases from the 3′ end of the Acu1 tagging primer.
65. The method of item 63 or 64, wherein the Acu1 tagging primer comprises a detection handle positioned at the 5′ end of the Acu1 tagging primer.
66. The method of item 65, wherein the detection handle comprises or consists of the sequence 5′-GCAATTCCTCACGAGACCCGTCCTG-3′ (SEQ ID NO: 53).
67. The method of any one of items 63 to 66, wherein the infectious agent is SARS CoV2, Deltavirus, Adenoviridae, Anelloviridae, Arenaviridae, Astroviridae, Caliciviridae, Coronaviridae, Filoviridae, Flaviviridae, Hantaviridae, Hepadnaviridae, Hepeviridae, Herpesviridae, Nairoviridae, Orthomyxoviridae, Papillomaviridae, Paramyxoviridae, Parvoviridae, Peribunyaviridae, Phenuviridae, Picornaviridae, Pneumoviridae, Polyomaviridae, Poxviridae, Reoviridae, Retroviridae, Rhabdoviridae, Smacoviridae, or Togaviridae.
68. The method of any one of items 63 item 67, wherein the reaction conditions of step (b) are carried out for about 10 minutes at room temperature.
69. The method of item 63 to 68, wherein the heat inactivation step comprises heating for about 1 minute at about 65° C.
70. The method of any one of items 63 to 69, wherein the at least one primer polynucleotide further comprises a quencher and said one or more variant adaptors comprise a fluorophore.
71. A method of detecting a dinucleotide sequence in a target sequence of an infectious agent polynucleotide sample, the method comprising:
(a) contacting the target polynucleotide containing sample with (i) at least one Acu1 tagging primer, and (ii) a at least one reverse Acu1 primer, under condition to generate an Acu1-tagged amplicon;
(b) subjecting the Acu1-tagged amplicon to (i) a digestion with Acu1 to generate a digestion reaction mixture comprising a digested Acu1-tagged amplicon, and (ii) a one or more variant adaptors under condition to ligate said one or more adaptors to the digested Acu1-tagged amplicon, to generate a ligate product;
(c) subjecting the digestion reaction mixture to a heat inactivation step to generate a heat inactivation reaction mixture; and
(d) detecting said ligated product.
72. The method of item 71, wherein said Acu1 tagging primer comprises an Acu1 motif polynucleotide (5′-CTGAAG-3′) positioned 14 bases from the 3′ end of the Acu1 tagging primer.
73. The method of item 71 or 72, wherein the Acu1 tagging primer comprises a detection handle positioned at the 5′ end of the Acu1 tagging primer.
74. The method of any one of items 71 to 73, wherein the infectious agent is SARS CoV2, Deltavirus, Adenoviridae, Anelloviridae, Arenaviridae, Astroviridae, Caliciviridae, Coronaviridae, Filoviridae, Flaviviridae, Hantaviridae, Hepadnaviridae, Hepeviridae, Herpesviridae, Nairoviridae, Orthomyxoviridae, Papillomaviridae, Paramyxoviridae, Parvoviridae, Peribunyaviridae, Phenuviridae, Picornaviridae, Pneumoviridae, Polyomaviridae, Poxviridae, Reoviridae, Retroviridae, Rhabdoviridae, Smacoviridae, or Togaviridae.
75. The method of any one of items 71 to 74, wherein the ligase if a T4 ligase or a T3 ligase.
76. The method of item 75, wherein the T4 ligase is a heat resistant (Hi-T4) T4 ligase, a salt-tolerant (Salt-T4) T4 ligase or, a highly concentrated (T4-HC) T4 ligase.
77. The method of any one of items 71 to 76, wherein the reaction temperature is between about 16° C. and about 37° C.
78. The method of any one of items 71 to 76, wherein the reaction temperature is about room temperature.
79. The method of any one of items 71 to 78, wherein the reaction time is between 1 min to 1 hour.
80. A method of detecting a dinucleotide sequence in a target polynucleotide containing sample from a subject, the method comprising
(a) contacting the target polynucleotide containing sample with (i) at least one Acu1 tagging primer, and (ii) a at least one reverse Acu1 primer, under condition to generate an Acu1-tagged amplicon;
(b) contacting the Acu1-tagged amplicon with Acu1, one or more variant adaptors at a concentration of about 250 uM, and a ligase, to generate a reaction mixture,
(c) subjecting the reaction mixture to a reaction time and reaction temperature, to generate a ligation product, and
(d) detecting said ligated product.
81. The method of item 80, further comprising a competitor DNA, optionally OB196 5′-AGCCTGTGGTTCCTGAAGATCGCGTCCGAT-3′ (SEQ ID NO: 59) or OB197 5′-ATCGGACGCGATCTTCAGGAACCACAGGCT-3′ (SEQ ID NO: 60).
82. A method of detecting a dinucleotide sequence in a target polynucleotide containing sample from a subject, the method comprising:
(a) contacting the target polynucleotide containing sample with (i) at least one Acu1 tagging primer, and (ii) a at least one reverse Acu1 primer, under condition to generate an Acu1-tagged amplicon;
(b) contacting the Acu1-tagged amplicon with Acu1, one or more variant adaptors at a concentration of about 250 uM, a competitor DNA, optionally OB196 5′-AGCCTGTGGTTCCTGAAGATCGCGTCCGAT-3′ (SEQ ID NO: 59) or OB197 5′-ATCGGACGCGATCTTCAGGAACCACAGGCT-3′ (SEQ ID NO: 60), and a ligase, to generate a reaction mixture, and
(c) subjecting the reaction mixture to a reaction time and reaction temperature, to generate a ligation product, and
(d) detecting said ligated product.
83. The method of any one of items 80 to 82, wherein said Acu1 tagging primer comprises an Acu1 motif polynucleotide (5′-CTGAAG-3′) positioned 14 bases from the 3′ end of the Acu1 tagging primer.
84. The method of any one of items 80 to 83, wherein the Acu1 tagging primer comprises a detection handle positioned at the 5′ end of the Acu1 tagging primer.
85. The method of item 84, wherein the detection handle comprises or consists of the sequence 5′-GCAATTCCTCACGAGACCCGTCCTG-3′ (SEQ ID NO: 53).
86. The method of anyone of items 81 to 85, wherein the concentration of the competitor DNA is about 1 pmol.
87. The method of any one of items 81 to 86, wherein the ligase is a T4 ligase or a T3 ligase
88. The method of item 87, wherein the T4 ligase is a heat resistant (Hi-T4) T4 ligase, a salt-tolerant (Salt-T4) T4 ligase or, a highly concentrated (T4-HC) T4 ligase.
89. The method of any one of items 81 to 88, wherein said detecting is quantitative, semi-quantitative, analytical, or visual.
90. The method of any one of items 81 to 89, wherein the sample is from a eukaryote, a prokaryote, or a virus.
91. The method of any one of items 81 to 89, wherein the subject is a mammal, a plant, a bacterium, a fungus, a protest, or a virus.
92. The method of any one of items 81 to 91, wherein the sample is isolated from a cell, a cell pellet, a cell extract, a tissue, a biopsy, or biological fluid, obtained from the subject.
93. The method of any one of items 81 to 92, wherein the target polynucleotide is a severe acute respiratory syndrome coronavirus 2 (SARS-CoV-2) polynucleotide sample.
93. The method of any one of items 81 to 92, wherein the target polynucleotide is the PIK3R1 gene, a DNA repair gene, or PCNA.
94. The method of any one of items 81 to 93, wherein the dinucleotide is a mutation, or a reference sequence.
95. The method of item 94, wherein the mutation is a transition, transversion, insertion, or deletion.
96. The method of any one of items 81 to 95, wherein the sample is from a cancer sample.
97. The method of any one of items 81 to 96, wherein the sample is from a nasal swab, a sample from an oropharyngeal swab, a sputum sample, a lower respiratory tract aspirate, a bronchoalveolar lavage, a nasopharyngeal wash/aspirate or a nasal aspirate.
98. The method of any one of items 81 to 97, wherein the subject is a human.
99. A method of detecting a dinucleotide sequence in a target polynucleotide containing sample from a subject, the method comprising
(a) contacting the target polynucleotide containing sample with (i) at least one LAMP Acu1 tagging primer, and (ii) a at least one reverse LAMP Acu1 primer, under condition to generate a LAMP Acu1-tagged amplicon;
(b) contacting the LAMP Acu1-tagged amplicon with Acu1, one or more LAMP-specific adaptors, and a ligase, to generate a reaction mixture,
(c) subjecting the reaction mixture to a reaction time and reaction temperature, to generate a LAMP ligation product, and
(d) detecting said LAMP ligated product.
100. The method of item 99, wherein said LAMP-Acu1 tagging primer comprises an Acu1 motif polynucleotide (5′-CTGAAG-3′) positioned 14 bases from the 3′ end of the Acu1 tagging primer.
101. The method of item 99 or 100, wherein the Acu1 tagging primer comprises a F2 and F3 LAMP sequence at the 5′ end of the LAMP Acu1 tagging primer.
102. The method of any one of items 99 to 101, wherein the ligase if a T4 ligase or a T3 ligase.
103. The method of item 102, wherein the T4 ligase is a heat resistant (Hi-T4) T4 ligase, a salt-tolerant (Salt-T4) T4 ligase or, a highly concentrated (T4-HC) T4 ligase.
104. The method of any one of items 99 to 103, wherein the reaction temperature is between about 16° C. and about 37° C.
105. The method of any one of items 99 to 104, wherein the reaction temperature is about room temperature.
106. The method of any one of items 99 to 105, wherein the reaction time is between 1 min to 1 hour.
107. The method of any one of items 99 to 106, wherein said detecting is quantitative, semi-quantitative, analytical, or visual.
108. The method of any one of items 99 to 106, wherein the sample is from a eukaryote, a prokaryote, or a virus.
109. The method of any one of items 99 to 106, wherein the subject is a mammal, a plant, a bacterium, a fungus, a protest, or a virus.
109. The method of any one of items 99 to 109, wherein the sample is isolated from a cell, a cell pellet, a cell extract, a tissue, a biopsy, or biological fluid, obtained from the subject.
110. The method of any one of items 99 to 109, wherein the target polynucleotide is a severe acute respiratory syndrome coronavirus 2 (SARS-CoV-2) polynucleotide sample.
111. The method of any one of items 99 to 112, wherein the target polynucleotide is the PIK3R1 gene, a DNA repair gene, or PCNA.
112. The method of any one of items 99 to 111, wherein the dinucleotide is a mutation, or a reference sequence.
113. The method of item 112, wherein the mutation is a transition, transversion, insertion, or deletion.
114. The method of any one of items 99 to 113, wherein the sample is from a cancer sample.
115. The method of any one of items 99 to 114, wherein the sample is from a nasal swab, a sample from an oropharyngeal swab, a sputum sample, a lower respiratory tract aspirate, a bronchoalveolar lavage, a nasopharyngeal wash/aspirate or a nasal aspirate.
116. The method of any one of items 99 to 115, wherein the subject is a human.
117. A kit comprising one or more isolated polynucleotide selected from one or more of: SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO:70, SEQ ID NO: 71, SEQ ID NO: 72, SEQ ID NO: 73, SEQ ID NO: 74, SEQ ID NO: 75, SEQ ID NO: 76, SEQ ID NO:77, SEQ ID NO: 78, SEQ ID NO: 79, SEQ ID NO:80, SEQ ID NO: 81 SEQ ID NO: 82, SEQ ID NO: 83, SEQ ID NO: 84, SEQ ID NO:85, and a container, and optionally instructions for the use thereof.

In one aspect there is provided a method of detecting a mutation in a target sequence of a severe acute respiratory syndrome coronavirus 2 (SARS-CoV-2) polynucleotide sample, the method comprising:
(a) contacting a SARS-CoV-2 polynucleotide containing sample with at least one primer polynucleotide comprising, or consisting of, a structure of formula (I):

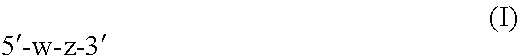

wherein w comprises or consists of an Acu1-tagging primer polynucleotide, and z comprises or consists of a target sequence polynucleotide, wherein w comprises or consists of a structure of formula (II).

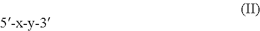

wherein x comprises or consists of an Acu1-handle polynucleotide and y comprises or consists of an Acu1 motif polynucleotide, under condition to generate an Acu1-tagged amplicon;
(b) subjecting the Acu1-tagged amplicon to a digestion with Acu1 to generate a digestion reaction mixture comprising a digested Acu1-tagged amplicon;
(c) subjecting the digestion reaction mixture to a heat inactivation step to generate a heat inactivation reaction mixture;
(d) contacting the heat inactivation reaction mixture with a one or more variant adaptors under conditions to ligate said one or more adaptors to the digested Acu1-tagged amplicon, to generate a ligation product; and (e) detecting said ligated product.

In one example, wherein subjecting the Acu1-tagged amplicon to a digestion with Acu1 to generate a digestion reaction mixture comprising a digested Acu1-tagged amplicon is for about 1 minute at about 37° C.

In one example, wherein said heat inactivation steps comprises heating for about 1 minute to about 10 minutes at about 65° C.

In one example, wherein step (d) comprises a first step for about 10 minutes at about 65° C., a heating step for about 10 minutes.

In one example, the conditions to ligate said one or more adaptors comprises using T4 ligase or T3 ligase

In one aspect there is provided a method of detecting a mutation in a target sequence of a severe acute respiratory syndrome coronavirus 2 (SARS-CoV-2) polynucleotide sample, the method comprising:
(a) contacting a SARS-CoV-2 polynucleotide containing sample with at least one primer polynucleotide comprising, or consisting of, a structure of formula (I):

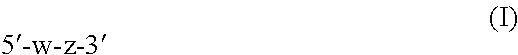

wherein w comprises or consists of an Acu1-tagging primer polynucleotide, and z comprises or consists of a target sequence polynucleotide;
wherein w comprises or consists of a structure of formula (II).

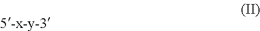

wherein x comprises or consists of an Acu1-handle polynucleotide and y comprises or consists of an Acu1 motif polynucleotide, under condition to generate an Acu1-tagged amplicon;
(b) subjecting the Acu1-tagged amplicon to (i) a digestion with Acu1 to generate a digestion reaction mixture comprising a digested Acu1-tagged amplicon, and (ii) a one or more variant adaptors under condition to ligate said one or more adaptors to the digested Acu1-tagged amplicon, to generate a ligation product;
(c) subjecting the digestion reaction mixture to a heat inactivation step to generate a heat inactivation reaction mixture;
(d) detecting said ligated product.

In one example, wherein the reaction conditions of step (b) are carried out for about 10 minutes at room temperature.

In one example, the heat inactivation step comprises heating for about 1 minute at about 65 C.
9. A method of detecting a severe acute respiratory syndrome coronavirus in a sample, the method comprising:
(a) contacting a SARS-CoV-2 polynucleotide containing sample with at least one primer polynucleotide comprising, or consisting of, a structure of formula (I):

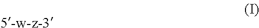

wherein w comprises or consists of an Acu1-tagging primer polynucleotide, and z comprises or consists of a target sequence polynucleotide,
wherein w comprises or consists of a structure of formula (II).

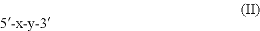

wherein x comprises or consists of an Acu1-handle polynucleotide and y comprises or consists of an Acu1 motif polynucleotide, under condition to generate an Acu1-tagged amplicon;
(b) subjecting the Acu1-tagged amplicon to (i) a digestion with Acu1 to generate a digestion reaction mixture comprising a digested Acu1-tagged amplicon, and (ii) a one or more variant adaptors under condition to ligate said one or more adaptors to the digested Acu1-tagged amplicon, to generate a ligate product;
(c) subjecting the digestion reaction mixture to a heat inactivation step to generate a heat inactivation reaction mixture;
(d) detecting said ligated product.

In one example, wherein the reaction conditions of step (b) are carried out for about 10 minutes at room temperature.

In one example, wherein the heat inactivation step comprises heating for about 1 minute at about 65° C.

In one example, wherein the at least one primer polynucleotide further comprises a quencher and said one or more variant adaptors comprise a fluorophore.

In one aspect there is provide a kit comprising an adaptor, a container, and optionally instructions for the use thereof, said adaptor comprising a double-stranded DNA formed by the annealing of two complementary oligonucleotide; one of the two strand contains a 3′ dinucleotide overhang that is used to capture the complementary variant signature.

In one aspect there is provided a kit comprising one or more isolated polynucleotide selected from:

| | | | |
|---|---|---|---|
| OB1 | Reverse adaptor | CTGGGGCACGGGTAAGAAGCATTCTGTCTCTCTTCTAAgaattcgagctcggtacccg | 1 |
| OB2 | AA-DTECT Adaptor | cgggtaccgagctcgaattcTTAGAAGAGAGACAGAATGCTTCTTACCCGTGCCCCAGAA | 2 |
| OB3 | AC-DTECT Adaptor | cgggtaccgagctcgaattcTTAGAAGAGAGACAGAATGCTTCTTACCCGTGCCCCAGAC | 3 |
| OB4 | AG-DTECT Adaptor | cgggtaccgagctcgaattcTTAGAAGAGAGACAGAATGCTTCTTACCCGTGCCCCAGAG | 4 |
| OB5 | AT-DTECT Adaptor | cgggtaccgagctcgaattcTTAGAAGAGAGACAGAATGCTTCTTACCCGTGCCCCAGAT | 5 |
| OB6 | CA-DTECT Adaptor | cgggtaccgagctcgaattcTTAGAAGAGAGACAGAATGCTTCTTACCCGTGCCCCAGCA | 6 |
| OB7 | CC-DTECT Adaptor | cgggtaccgagctcgaattcTTAGAAGAGAGACAGAATGCTTCTTACCCGTGCCCCAGCC | 7 |
| OB8 | CG-DTECT Adaptor | cgggtaccgagctcgaattcTTAGAAGAGAGACAGAATGCTTCTTACCCGTGCCCCAGCG | 8 |
| OB9 | CT-DTECT Adaptor | cgggtaccgagctcgaattcTTAGAAGAGAGACAGAATGCTTCTTACCCGTGCCCCAGCT | 9 |
| OB10 | GA-DTECT Adaptor | cgggtaccgagctcgaattcTTAGAAGAGAGACAGAATGCTTCTTACCCGTGCCCCAGGA | 10 |
| OB11 | GC-DTECT Adaptor | cgggtaccgagctcgaattcTTAGAAGAGAGACAGAATGCTTCTTACCCGTGCCCCAGGC | 11 |
| OB12 | GG-DTECT Adaptor | cgggtaccgagctcgaattcTTAGAAGAGAGACAGAATGCTTCTTACCCGTGCCCCAGGG | 12 |
| OB13 | GT-DTECT Adaptor | cgggtaccgagctcgaattcTTAGAAGAGAGACAGAATGCTTCTTACCCGTGCCCCAGGT | 13 |
| OB14 | TA-DTECT Adaptor | cgggtaccgagctcgaattcTTAGAAGAGAGACAGAATGCTTCTTACCCGTGCCCCAGTA | 14 |
| OB15 | TC-DTECT Adaptor | cgggtaccgagctcgaattcTTAGAAGAGAGACAGAATGCTTCTTACCCGTGCCCCAGTC | 15 |
| OB16 | TG-DTECT Adaptor | cgggtaccgagctcgaattcTTAGAAGAGAGACAGAATGCTTCTTACCCGTGCCCCAGTG | 16 |
| or | | | |
| OB17 | TT-DTECT Adaptor | cgggtaccgagctcgaattcTTAGAAGAGAGACAGAATGCTTCTTACCCGTGCCCCAGTT | 17 |

and a container, and optionally instructions for use thereof.

In one aspect there is provided a method for detecting a mutation in a target sequence of an infectious agent polynucleotide sample, the method comprising:
(a) contacting a SARS-CoV-2 polynucleotide containing sample with at least one primer polynucleotide comprising, or consisting of, a structure of formula (I):

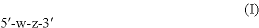

wherein w comprises or consists of an Acu1-tagging primer polynucleotide, and z comprises or consists of a target sequence polynucleotide,
wherein w comprises or consists of a structure of formula (II).

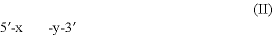

wherein x comprises or consists of an Acu1-handle polynucleotide and y comprises or consists of an Acu1 motif polynucleotide, under condition to generate an Acu1-tagged amplicon;
(b) subjecting the Acu1-tagged amplicon to a digestion with Acu1 to generate a digestion reaction mixture comprising a digested Acu1-tagged amplicon;
(c) subjecting the digestion reaction mixture to a heat inactivation step to generate a heat inactivation reaction mixture;
(d) contacting the heat inactivation reaction mixture with a one or more variant adaptors under conditions to ligate said one or more adaptors to the digested Acu1-tagged amplicon, to generate a ligation product; and (e) detecting said ligated product.

In one example, wherein the infectious agent is Deltavirus, Adenoviridae, Anelloviridae, Arenaviridae, Astroviridae, Caliciviridae, Coronaviridae, Filoviridae, Flaviviridae, Hantaviridae, Hepadnaviridae, Hepeviridae, Herpesviridae, Nairoviridae, Orthomyxoviridae, Papillomaviridae, Paramyxoviridae, Parvoviridae, Peribunyaviridae, Phenuviridae, Picornaviridae, Pneumoviridae, Polyomaviridae, Poxviridae, Reoviridae, Retroviridae, Rhabdoviridae, Smacoviridae, or Togaviridae

In one example, wherein subjecting the Acu1-tagged amplicon to a digestion with Acu1 to generate a digestion reaction mixture comprising a digested Acu1-tagged amplicon is for about 1 minute at about 37° C.

In one example, wherein said heat inactivation steps comprises heating for about 1 minute to about 10 minutes at about 65° C.

In one example, wherein step (d) comprises a first step for about 10 minutes at about 65° C., a heating step for about 10 minutes.

In one example, wherein the conditions to ligate said one or more adaptors comprises using T4 ligase or T3 ligase

In one aspect there is provided a method for detecting a mutation in a target sequence of an infectious agent polynucleotide sample, the method comprising:
(a) contacting a SARS-CoV-2 polynucleotide containing sample with at least one primer polynucleotide comprising, or consisting of, a structure of formula (I):

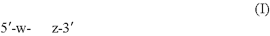

wherein w comprises or consists of an Acu1-tagging primer polynucleotide, and z comprises or consists of a target sequence polynucleotide;
wherein w comprises or consists of a structure of formula (II).

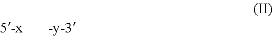

wherein x comprises or consists of an Acu1-handle polynucleotide and y comprises or consists of an Acu1 motif polynucleotide, under condition to generate an Acu1-tagged amplicon;
(b) subjecting the Acu1-tagged amplicon to (i) a digestion with Acu1 to generate a digestion reaction mixture comprising a digested Acu1-tagged amplicon, and (ii) a one or more variant adaptors under condition to ligate said one or more adaptors to the digested Acu1-tagged amplicon, to generate a ligate product;
(c) subjecting the digestion reaction mixture to a heat inactivation step to generate a heat inactivation reaction mixture;
(d) detecting said ligated product.

In one example, wherein the infectious agent is Deltavirus, Adenoviridae, Anelloviridae, Arenaviridae, Astroviridae, Caliciviridae, Coronaviridae, Filoviridae, Flaviviridae, Hantaviridae, Hepadnaviridae, Hepeviridae, Herpesviridae, Nairoviridae, Orthomyxoviridae, Papillomaviridae, Paramyxoviridae, Parvoviridae, Peribunyaviridae, Phenuviridae, Picornaviridae, Pneumoviridae, Polyomaviridae, Poxviridae, Reoviridae, Retroviridae, Rhabdoviridae, Smacoviridae, or Togaviridae

In one example, wherein the reaction conditions of step (b) are carried out for about 10 minutes at room temperature.

In one example, wherein the heat inactivation step comprises heating for about 1 minute at about 65 C.

In one aspect there is provided a method for detecting a mutation in a target sequence of an infectious agent polynucleotide sample, the method comprising:
(a) contacting a SARS-CoV-2 polynucleotide containing sample with at least one primer polynucleotide comprising, or consisting of, a structure of formula (I):

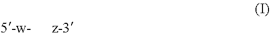

wherein w comprises or consists of an Acu1-tagging primer polynucleotide,
and z comprises or consists of a target sequence polynucleotide,
wherein w comprises or consists of a structure of formula (II).

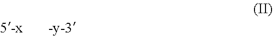

wherein x comprises or consists of an Acu1-handle polynucleotide and y comprises or consists of an Acu1 motif polynucleotide, under condition to generate an Acu1-tagged amplicon;
(b) subjecting the Acu1-tagged amplicon to (i) a digestion with Acu1 to generate a digestion reaction mixture comprising a digested Acu1-tagged amplicon, and (ii) a one or more variant adaptors under condition to ligate said one or more adaptors to the digested Acu1-tagged amplicon, to generate a ligate product;
(c) subjecting the digestion reaction mixture to a heat inactivation step to generate a heat inactivation reaction mixture;
(d) detecting said ligated product.

In one example, wherein the infectious agent is Deltavirus, Adenoviridae, Anelloviridae, Arenaviridae, Astroviridae, Caliciviridae, Coronaviridae, Filoviridae, Flaviviridae, Hantaviridae, Hepadnaviridae, Hepeviridae, Herpesviridae, Nairoviridae, Orthomyxoviridae, Papillomaviridae, Paramyxoviridae, Parvoviridae, Peribunyaviridae, Phenuviridae, Picornaviridae, Pneumoviridae, Polyomaviridae, Poxviridae, Reoviridae, Retroviridae, Rhabdoviridae, Smacoviridae, or Togaviridae

In one example, wherein the reaction conditions of step (b) are carried out for about 10 minutes at room temperature.

In one example, wherein the heat inactivation step comprises heating for about 1 minute at about 65° C.

In one example, wherein the at least one primer polynucleotide further comprises a quencher and said one or more variant adaptors comprise a fluorophore.

### BRIEF DESCRIPTION OF THE DRAWINGS

Embodiments of the present disclosure will now be described, by way of example only, with reference to the attached Figures.

FIG. 1. DTECT efficiently captures SARS-CoV-2 signatures. A) Illustration of the various steps of DTECT1.0. SARS-CoV-2 signatures (targeted dinucleotide) are amplified by PCR (Step 1) using an Acu1-tagging primer. The Acu1-tagging primer includes a hairpin (green) that encodes the Acu1 motif. The Acu1-tagging primer is juxtaposed to the signature, so the reference and variant signatures do not compete, like in variant-specific PCR approaches. The PCR product is subsequently digested by the Acu1 endonuclease (Step 2) to generate two DNA fragments and expose SARSCoV-2 signatures. The smaller fragment (60 bp) containing the exposed signature of the targeted dinucleotide is then isolated (Step 3) to decrease potential interference between the digestion fragments and adaptors during ligation. The exposed signature is then ligated to DNA adaptors (Step 4) containing 3′ overhangs of two bases complementary (specific) or not (non-specific) to the dinucleotide signature. The use of non-complementary adaptors validates the specificity of the detection. Finally, the ligated product is analyzed by PCR for analytical or quantitative detection (Step 5) using a unique pair of oligos that are complementary to the Acu1 handle (blue sequence in Acu1-tagging oligo—step 1) and to the adaptors. The detection either provides a quantitative assessment of the different populations of variants or a rapid determination of the presence/absence of variants. B) Heat map representing the relative frequency of the capture of the reference and variant signatures in the reference SARS-CoV-2 sequence of three different lineages (B.1.1.7, B.1.351, and P.1). Each capture has been performed in independent duplicates. C) Quantification of various SARS-CoV-2 variants (green circles) and references (red circles) using the reference (left) or variants (right) sequences. The mean specificity capture and Mean background capture are shown for nine independent captures performed in independent duplicates. D) depicts methods of the present disclosure, also referred to as DETECT2.0.

FIG. 2. Accelerated DTECT2.0
A) Schematic representation of the steps required to capture signatures with DTECT1.0. Acu1-tagged amplicon is digested with Acu1 (pale blue) followed by Acu1 inactivation (orange), the small fragment of digestion is isolated with beads (purple) and ligated to adaptors by a DNA ligase (dark blue). B) Analytical representation of SARS-CoV-2 E484K signature capture using the variant-specific adaptor (top). Quantification of the capture with the variant-specific adaptor (green circles) and reference-specific adaptor (red triangles). DTECT1.0 was conducted with or without Acu1, beads isolation, and DNA ligase (middle). Quantification of the capture specificity in with or without Acu1, beads isolation and DNA ligase (bottom). Error bars represent s.e.m of two independent experiments. C) Time course of Acu1 digestion. The Acu1-tagged amplicon was digested with Acu1 for the indicated time. The specificity score is shown. Error bars represent s.e.m of two independent experiments. Original conditions utilized in DTECT1.0 are shown by the arrow. D) Time course inactivation of Acu1 by heating at 65° C. for the indicated time. The specificity score is shown. Error bars represent s.e.m of two independent experiments. Original conditions utilized in DTECT1.0 are shown by the arrow. E) Measure of the specificity score with the indicated concentration of ligase. The specificity score is shown. Error bars represent s.e.m of two independent experiments. Original conditions utilized in DTECT1.0 are shown by the arrow. F) Measure of the specificity score with the indicated concentration of adaptors. The specificity score is shown. Error bars represent s.e.m of two independent experiments. The arrow indicates original conditions utilized in DTECT1.0. G) Time course of ligation. DNA ligation was incubated at 25° C. for the indicated time. The specificity score is shown. Error bars represent s.e.m of two independent experiments. Original conditions utilized in DTECT1.0 are shown by the arrow.

FIG. 3. A sensitive single-step DTECT2.0. A) Schematic representation of the one-pot/single-step capture (up). Quantification of the specificity score using DTECT1.0 or one-pot reaction (middle). Quantification of the capture with the variant-specific adaptor (green circles) and reference-specific adaptor (red triangles) using DTECT1.0 or one-pot reaction. No Acu1 or ligase reactions are used as control (bottom). Error bars represent s.e.m of two independent experiments. B) Quantification of the specificity score using one-pot reaction with various DNA ligases, as indicated. Each ligation was conducted in ligase-specific buffers at 25° C. (T4, T3, T7, T4 HC, Hi-T4 and Salt) or 45° C. (9N, Taq, and HiFi Taq). Heat resistant ligases (9N, Taq, and HiFi Taq) were immediately loaded into the qPCR for analysis. A no ligase reaction is used as a control. Inv=Invitrogen, NEB=New England Biolabs. Error bars represent s.e.m of two independent experiments. C) Quantification of the specificity score using one-pot reaction and increasing quantity of Acu1, as indicated. Digestion was conducted at 25° C. for 1 min followed by 1 min at 65° C. to heat inactivate. A no ligase reaction is used as a control. Inv=Invitrogen, NEB=New England Biolabs. Error bars represent s.e.m of two independent experiments. D) Quantification of the specificity score for single pot reactions either as two steps or single-step digestion-ligation. The digestion reaction was either conducted at 25° C. or 37° C., for 1 min as indicated. Ligation was conducted at 25° C. for 10 min followed by heat inactivation for 1 min at 65° C. No Acu1 reactions are used as a control. Error bars represent s.e.m of two independent experiments. E) Schematic representation of the differences between DTECT1.0, DTECT2.0, and single-step DTECT2.0. The different steps are shown in different colors.

FIG. 4. Determination of the capture score for Acu1 digestion kinetics (A), Acu1 heat inactivation kinetics (B), titration of adaptors (C), DNA ligation kinetics (D), and concentration of T4 ligase (E).

FIG. 5. The capture score was calculated for the different ligases (A), the concentration of Acu1 (B), and for single pot reactions either as two steps or single-step digestion-ligation (E). Specificity and capture scores were calculated for adaptors (C) and ligase (D) concentration titrations.

FIG. 6. Schematic representation of DTECT. a) Illustration of the Acu1 tagging steps of DTECTv1. A locus of interest is amplified by PCR (Step 1). The PCR is conducted using an “Acu1-tagging primer” containing an Acu1 hairpin (green) that encodes the Acu1 motif. The Acu1-tagging primer is juxtaposed to the signature, so the reference and variant signatures do not compete, like in variant-specific PCR approaches. The oligonucleotide also contains a detection handle in its 5′ end. A regular reverse oligonucleotide complementary to the genomic sequence is also used to obtain the Acu1 tagged amplicon (right), which contains the detection handle, Acu1 motif, and the genomic sequence with the dinucleotide of interest. b) Illustration of the signature capture, which contains three steps: Acu1 digestion, fragment isolation, and adaptor ligation. The PCR product generated in step 1 is digested by the Acu1 endonuclease (Step 2) to generate two DNA fragments and expose the dinucleotide signature of interest. The small fragment (60 bp) containing the exposed signature of the targeted dinucleotide is then isolated (Step 3) to decrease potential interference between the digestion fragments and adaptors during ligation. The exposed signature is then ligated to DNA adaptors (Step 4) containing 3′ overhangs of two bases complementary (specific, in blue) or not (non-specific, in brown) to the dinucleotide signature. The use of non-complementary adaptors validates the specificity of the detection. c) The ligated product is amplified using the detection primers 1 and 2 and either analyzed by analytical or quantitative PCR (Step 5). The unique pair of detection primers are complementary to the Acu1 handle (red sequence in Acu1-tagging primer—step 1) and the adaptors. The detection either provides a quantitative assessment of the different populations of variants or a rapid determination of the presence/absence of variants.

FIG. 7. Development of an accelerated DTECTv2. a) Schematic representation of the steps required for signature capture with DTECTv1. Acu1-tagged amplicon is digested with Acu1 (step I—pale blue), followed by Acu1 heat inactivation (step II—orange), the small digested fragment is isolated with beads (step III—dark blue), and adaptors are ligated with a DNA ligase (step IV—purple). b) DTECTv1 was conducted by omitting the indicated step/enzyme. Capture specificity was measured by qPCR. Error bars represent s.d of two independent experiments. c) DTECTv1 was conducted by omitting the indicated step/enzyme. Capture efficiency using specific (in green) or non-specific (red) adaptors was measured by qPCR. Error bars represent s.d of two independent experiments. d) Acu1 digestion kinetics. The Acu1-tagged amplicon was digested with Acu1 for the indicated time and captured using specific (in green) or non-specific (red) adaptors. The capture efficiency was determined by qPCR. Error bars represent s.d of two independent experiments. Original conditions utilized in DTECTv1 are shown by the blue arrow, and the green arrow indicates the negative control without Acu1. e) Acu1 heat inactivation kinetics. Acu1 was heat-inactivated by incubating at 65° C. for the indicated time. Then, the Acu1-tagged amplicon was added to start the digestion. The capture efficiency was determined by qPCR. Error bars represent s.d of two independent experiments. The blue arrow shows the original conditions utilized in DTECTv1, and the positive control in which Acu1 was not pre-inactivated is indicated with the green arrow. f) Adaptor ligation kinetics. DNA ligation was incubated at 25° C. for the indicated time. The capture efficiency was determined by qPCR. Error bars represent s.d of two independent experiments. Original conditions utilized in DTECTv1 are shown by the blue arrow, and the green arrow indicates negative control without ligase. g) Schematic representation of the differences between DTECTv1 (top) and DTECTv2 (bottom). DTECTv1 includes a beads isolation step and extended incubation times. h) Comparison of the capture efficiency for the capture of the E484K SARS-CoV-2 variant using the specific (green) or non-specific adaptor (red) with DTECTv1 or DTECTv2. i) Comparison of the capture efficiency for the capture of the PIK3R1 reference (WT) and PIK3R1 variant (STOP) using the respective specific (green and light green) or non-specific (red and orange) adaptors with DTECTv1 or DTECTv2.

FIG. 8. Development of a sensitive single pot all-in-one DTECTv3. a) Schematic representation of the capture using DTECTv2 (left—green) and single pot capture (right—orange). While DTECTv2 relies on two independent steps in two tubes, the single pot consists of the concomitant digestion-ligation all-in-one tube. b) Quantification of the capture specificity using DTECTv2 (green) or one-pot reaction (orange) using either 0.25 or 250 UM adaptors. Error bars represent s.d of two independent experiments. c) Quantification of the capture specificity using one-pot reaction with various DNA ligases, as indicated. Each ligation was conducted in ligase-specific buffers at 25° C. (T4, T3, T7, T4 HC, Hi-T4, and Salt) or 45° C. (9N, Taq, and HiFi Taq). Heat resistant ligases (9N, Taq, and HiFi Taq) were immediately loaded into the qPCR for analysis. A no ligase reaction is used as a control. Provider for T4 (1) is Invitrogen and for T4 (1) is NEB. Error bars represent s.d of two independent experiments. d) Buffer optimization of the single pot reaction. The capture specificity is measured by qPCR in each condition in which one element was omitted, as indicated. Error bars represent s.d of two independent experiments. e) Capture efficiency was measured after adding Acu1-specific or non-specific competitors. The use of a specific competitor is indicated by the “Acu1 motif” with the green circle for the specific adaptor and a red box for the non-specific adaptor. The use of non-specific competitors is indicated by “Control” with the green and red triangles for the specific and non-specific adaptors, respectively. The green and red arrows indicate the samples with no competitors. Error bars represent s.d of two independent experiments. f) Schematic representation of the optimizations that enhanced the single pot capture. g) Schematic representation of the DTECTv1, v2, and v3 procedures.

FIG. 9. DTECTv3 is highly accurate and quantitative. a) Capture efficiency of each of the 16 dinucleotide signatures using specific adaptors. Capture efficiency was measured by qPCR. Error bars represent s.d of two independent experiments. b) Capture efficiency of dinucleotides containing 0, 1 or 2 A/T. c) Capture efficiency of dinucleotide signatures according to their 5′ and 3′ nucleotide identity. d) Heat map showing the quantification of the relative capture frequency of PIK3R1 and SARS-CoV-2 WT and variant signatures in mixtures of WT and variant alleles at predefined ratios. e and f) Comparison of the mean frequency of WT and variant signatures determined by DTECTv3 in d.

FIG. 10. DTECTv3 detects base editing and prime editing efficiencies in human cells. a) Quantification using DTECTv3 of the editing efficiency by which 16 DNA repair genes were edited with base editing. Error bars represent s.d of two independent quantifications. b) Quantification using DTECTv3 of the editing efficiency of mutations introduced at the HEK3 locus edited in HEK293T. A small insertion (insCTT) and a small deletion (del1T) were introduced by prime editing. Error bars represent s.d of two independent quantifications. c) Crystal structure of human PCNA homotrimer (1AXC). The residues targeted with prime editing are indicated with the arrow and red circle (left). Quantification using DTECTv3 of the editing efficiency by which 6 PCNA cancer mutations are introduced in HEK293T cells by prime editing (right). The reference codon is shown above the arrow, and the prime edited codon is shown below the arrow. The edited nucleotides are highlighted in red. Error bars represent s.d of two independent quantifications. d) Genotyping of six clones edited with PCNA A96G. DTECTv3-based analytical PCR was used to identify the alleles present in the population using the CG adaptor to capture WT alleles and the GC adaptor to capture the mutated alleles. Gels are representative of two independent experiments. e) Sanger sequencing of the PCNA A96G clones shown in d to validate the DTECT results. Arrows indicate the edited nucleotides.

FIG. 11. DTECT-LAMP enables visual detection of genetic signatures. a) Schematic representation of DTECT-LAMP. DTECT-LAMP comprises three steps: First, Acu1 tagging with an Acu1-tagging oligo that contains the F2 and F3 LAMP sequences. Second, single-step capture using DTECTv3 with adaptors that contain the F1, B1, B2, and B3 LAMP sequences. Finally, the ligated product is detected by loop amplification by incubating the ligated product at 65° C. in a LAMP reaction. If ligation is successful, the color of the LAMP reaction is expected to turn yellow, as indicated. b) Detection of SARS-CoV-2 E484 reference and E484K variant using DTECT-LAMP. The TA adaptor captures the E484K variant, and the TG adaptor captures the E484 reference signature. Two independent couple of LAMP primers were used: SARS-CoV-2 ORF1a and geneN. Representative pictures of the detection are shown. c) Quantitative detection of DTECT-LAMP by measuring changes in optical density over time for the capture of the SARS-CoV-2 E484K variant using ORF1a LAMP oligonucleotides.

FIG. 12. DTECT efficiently captures SARS-CoV-2 signatures. a) Genomic sequences of the SARS-CoV-2 reference (Wuhan strain, green) and the 7 variant of concern reference (red) from the different SARS-CoV-2 strains, as indicated on the left. The dark line indicates the position of the codon encoding for the mutated amino acids. The arrow indicates the mutated nucleotide. The selected dinucleotide of interest is shown in the blue box, and the adaptors utilized for the capture of the reference and variant signatures are shown on the right. b) Qualitative detection of the indicated SARS-CoV-2 variants (red) and references (green) using the respective adaptors (reference in green, variant in red). Representative gels of two independent duplicates. c) Heat map representing the relative frequency of the reference and variant signatures captured from the reference SARS-CoV-2 genomic sequence or indicated variants. Each capture has been performed in two independent duplicates.

FIG. 13. Definition of the capture efficiency and specificity scores. a) Definition of the capture efficiency score. The amount of DNA captured is determined by reporting the measured Ct to the unique DTECTv3 standard curve. b) Definition of the capture specificity score. The capture specificity score represents the difference between Ct obtained by ligating specific and non-specific adaptors. The absolute value is a measure, so the capture specificity score is always positive.

FIG. 14. Development and enhancement of DTECTv3. a) Sanger sequencing reads of a ligation product obtained by capturing SARS-CoV-2 E484K variant with the single pot DTECTv3 using the TA adaptor. b) Quantification of the capture efficiency using one-pot reaction with various DNA ligases, as indicated. Refers to FIG. 3c. c) Capture specificity (left) and efficiency (right) using ligation enhancers. d) Quantification of the capture specificity using one-pot reaction using specific and non-specific competitors, as indicated. Refers to FIG. 3e. e) Capture efficiency (left) measured using specific (green) and non-specific (red) adaptors by incubating DTECTv3 at the indicated temperature. Capture specificity (right) was measured by incubating DTECTv3 at the indicated temperature. f) The Acu1-tagged PCR was either purified using three different protocols (gel, column-based, or bead-based purifications) or not purified.

FIG. 15. DTECTv3 enables accurate quantification of mixtures of WT and variant signatures. a) Quantification by DTECTv3 of the relative abundance of the indicated WT and variants in mixtures at predefined ratios.

### DETAILED DESCRIPTION

Coronaviruses are a large family of viruses which cause illness in animals or humans. In humans, several coronaviruses are known to cause respiratory infections ranging from the common cold to more severe diseases such as Middle East Respiratory Syndrome (MERS) and Severe Acute Respiratory Syndrome (SARS).

Most recently identified is the 2019 novel coronavirus (SARS-CoV-2 (SCoV2)/COVID-19).

Severe Acute Respiratory Coronavirus 2 (SARS-CoV-2), the causal agent of COVID-19, was characterized as a pandemic by the World Health Organization (WHO) in March 2020 and has triggered an international public health emergency

Globally, as of 6 Jun. 2021, there have been 172,630,637 confirmed cases of COVID-19, including 3,718,683 deaths, reported to WHO.

A number of variants of SARS-CoV-2 have been identified.

Variants are viruses that have changed or mutated. Variants are common with coronaviruses. A variant form may confer an evolutionary advantage or disadvantage relative to a progenitor form or may be neutral.

Mutations refers to nucleotide or amino acid substitutions, insertions or deletions, from the wild type (also referred to as reference) sequence. The term mutant or variants may encompass natural biological variants (e.g. allelic variants or geographical variations).

Thus, as used herein, the terms “variant polynucleotide” and “mutated polynucleotide” refer to one or more changes of a nucleic acid sequence of DNA or RNA, including, but not limited to a base substitution, insertion, deletion, reverse position, overlap, or the like

A SARS-CoV-2 isolate is a Variant of Interest (VOI) if, compared to a reference isolate, its genome has mutations with established or suspected phenotypic implications, and either: has been identified to cause community transmission/multiple COVID-19 cases/clusters, or has been detected in multiple countries; or is otherwise assessed to be a VOI by (for example) WHO in consultation with the WHO SARS-CoV-2 Virus Evolution Working Group.

As of 6 Jun. 2021, variants of interest include the following.

| | | | | Earliest | |
|---|---|---|---|---|---|
| WHO | | GISAID | Nextstrain | documented | Date of |
| label | Pango lineage | clade/lineage | clade | samples | designation |
| Epsilon | B.1.427/B.1.429 | GH/452R.V1 | 20C/S.452R | United | 5 Mar. |
| | | | | States of | 2021 |
| | | | | America, | |
| | | | | March 2020 | |
| Zeta | P.2 | GR | 20B/S.484K | Brazil, | 17 Mar. |
| | | | | April 2020 | 2021 |
| Eta | B.1.525 | G/484K.V3 | 20A/S484K | Multiple | 17 Mar. |
| | | | | countries, | 2021 |
| | | | | December 2020 | |
| Theta | P.3 | GR | 20B/S:265C | Philippines, | 24 Mar. |
| | | | | January 2021 | 2021 |
| Iota | B.1.526 | GH | 20C/S:484K | United States | 24 Mar. |
| | | | | of America, | 2021 |
| | | | | November 2020 | |
| Kappa | B.1.617.1 | G/452R.V3 | 21A/S:154K | India, | 4 Apr. |
| | | | | October 2020 | 2021 |

A SARS-CoV-2 variant of concern (VOC) is a variant that meets the definition of a VOI and, through a comparative assessment, has been demonstrated to be associated with one or more of the following changes at a degree of global public health significance: Increase in transmissibility or detrimental change in COVID-19 epidemiology; or Increase in virulence or change in clinical disease presentation; or Decrease in effectiveness of public health and social measures or available diagnostics, vaccines, therapeutics.

As of 6 Jun. 2021, variants concern include the following.

| | | | | Earliest | |
|---|---|---|---|---|---|
| WHO | Pango | GISAID | Nextstrain | documented | Date of |
| label | lineage | clade/lineage | clade | samples | designation |
| Alpha | B.1.1.7 | GRY | 20I:501Y.V1 | United | 18 Dec. |
| | | (formerly | | Kingdom, | 2020 |
| | | GR/501Y.V1) | | September 2020 | |
| Beta | B.1. 351 | GH/501Y.V2 | 20H/S:501Y.V2 | South Africa, | 18 Dec. |
| | | | | May 2020 | 2020 |
| Gamma | P.1 | GR/501Y.V3 | 20J/S:501Y.V3 | Brazil, | 11 Jan. |
| | | | | November 2020 | 2021 |
| Delta | B.1.617.2 | G/452R.V3 | 21A/S:478K | India, | VOI: 4 Apr. |
| | | | | October 2020 | 2021 |
| | | | | | VOC: 11 May |
| | | | | | 2021 |

Other naming systems are being developed for variants of SARS-CoV-2.

In one example, there is provided a method of detecting a mutation in a target sequence of a severe acute respiratory syndrome coronavirus 2 (SARS-CoV-2) polynucleotide sample, the method comprising:
(a) contacting a SARS-CoV-2 polynucleotide containing sample with at least one primer polynucleotide comprising, or consisting of, a structure of formula (I):

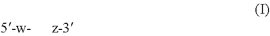

wherein w comprises or consists of an Acu1-tagging primer polynucleotide, and z comprises or consists of a target sequence polynucleotide,
wherein w comprises or consists of a structure of formula (II):

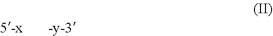

wherein x comprises or consists of an Acu1-handle polynucleotide and y comprises or consists of an Acu1 motif polynucleotide, under condition to generate an Acu1-tagged amplicon;
(b) subjecting the Acu1-tagged amplicon to a digestion with Acu1 to generate a digestion reaction mixture comprising a digested Acu1-tagged amplicon;
(c) subjecting the digestion reaction mixture to a heat inactivation step to generate a heat inactivation reaction mixture;
(d) contacting the heat inactivation reaction mixture with a one or more variant adaptors under conditions to ligate said one or more adaptors to the digested Acu1-tagged amplicon, to generate a ligation product; and (e) detecting said ligated product.

In one example, subjecting the Acu1-tagged amplicon to a digestion with Acu1 to generate a digestion reaction mixture comprising a digested Acu1-tagged amplicon is for about 1 minute at about 37° C.

In one example, said heat inactivation steps comprises heating for about 1 minute at about 65° C.

In one example, wherein step (d) comprises a first step for about 10 minutes at about 25° C., a heating step for about 10 minutes.

In one example, the conditions to ligate said one or more adaptors comprises using T4 ligase or T3 ligase

In one aspect there is provided a method of detecting a mutation in a target sequence of a severe acute respiratory syndrome coronavirus 2 (SARS-CoV-2) polynucleotide sample, the method comprising:
(a) contacting a SARS-CoV-2 polynucleotide containing sample with at least one primer polynucleotide comprising, or consisting of, a structure of formula (I):

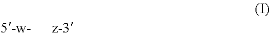

wherein w comprises or consists of an Acu1-tagging primer polynucleotide, and z comprises or consists of a target sequence polynucleotide;
wherein w comprises or consists of a structure of formula (II):

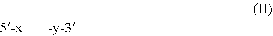

wherein x comprises or consists of an Acu1-handle polynucleotide and y comprises or consists of an Acu1 motif polynucleotide, under condition to generate an Acu1-tagged amplicon;
(b) subjecting the Acu1-tagged amplicon to (i) a digestion with Acu1 to generate a digestion reaction mixture comprising a digested Acu1-tagged amplicon, and (ii) a one or more variant adaptors under condition to ligate said one or more adaptors to the digested Acu1-tagged amplicon, to generate a ligate product;
(c) subjecting the digestion reaction mixture to a heat inactivation step to generate a heat inactivation reaction mixture;

In one example, the reaction conditions of step (b) are carried out for about 10 minutes at room temperature.

In one example, the heat inactivation step comprises heating for about 1 minute at about 65° C.

In one aspect there is provided a method of detecting a severe acute respiratory syndrome coronavirus in a sample, the method comprising:
(a) contacting a SARS-CoV-2 polynucleotide containing sample with at least one primer polynucleotide comprising, or consisting of, a structure of formula (I):

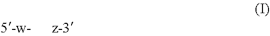

wherein w comprises or consists of an Acu1-tagging primer polynucleotide, and z comprises or consists of a target sequence polynucleotide,
wherein w comprises or consists of a structure of formula (II).

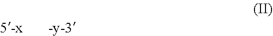

wherein x comprises or consists of an Acu1-handle polynucleotide and y comprises or consists of an Acu1 motif polynucleotide,
under condition to generate an Acu1-tagged amplicon;
(b) subjecting the Acu1-tagged amplicon to (i) a digestion with Acu1 to generate a digestion reaction mixture comprising a digested Acu1-tagged amplicon, and (ii) a one or more variant adaptors under condition to ligate said one or more adaptors to the digested Acu1-tagged amplicon, to generate a ligate product;
(c) subjecting the digestion reaction mixture to a heat inactivation step to generate a heat inactivation reaction mixture;
(d) detecting said ligated product.

In one example, wherein the reaction conditions of step (b) are carried out for about 10 minutes at room temperature.

In one example, the heat inactivation step comprises heating for about 1 minute at about 65° C.

In one example, wherein the at least one primer polynucleotide further comprises a quencher and said one or more variant adaptors comprise a fluorophore.

In another aspect, a Type IIS restriction enzyme-tagging primer polynucleotide is used. Specific examples of Type IIS restrictions enzymes include Acu1, Bpml, BpuEl, Bsgl, Mmel, and NMeAlll. In one aspect, there is provided a method for detecting a mutation in a target sequence of an infectious agent polynucleotide sample.

In some examples, the infectious agent is Deltavirus, Adenoviridae, Anelloviridae, Arenaviridae, Astroviridae, Caliciviridae, Coronaviridae, Filoviridae, Flaviviridae, Hantaviridae, Hepadnaviridae, Hepeviridae, Herpesviridae, Nairoviridae, Orthomyxoviridae, Papillomaviridae, Paramyxoviridae, Parvoviridae, Peribunyaviridae, Phenuviridae, Picornaviridae, Pneumoviridae, Polyomaviridae, Poxviridae, Reoviridae, Retroviridae, Rhabdoviridae, Smacoviridae, or Togaviridae

The term “detect” or “detecting” refers to identifying the presence, absence, or amount of the nucleic acid to be detected.

The term “mutation”, as used herein, refers to any change in a nucleic acid fragment relative to the “normal” (or wild type or reference) genetic material. The nucleotide sequence of the mutated nucleic acid herein displays one or more differences from the nucleotide sequence of the corresponding, non-mutated nucleic acid. A mutation may be one or more of a deletion, insertion, or substitution of one or more nucleotides.

The term “variants”, as used herein, includes nucleic acids and proteins whose sequence varies from the sequence of a reference nucleic acid and protein

Thus, in some examples a mutant may also be referred to as a variant.

The term “target sequence”, as used herein, refers to the region of interest on the original DNA. In some examples, the target sequence comprises the location(s) of the sequences of a VOI or VOC.

Accordingly, in some aspects, there is described herein a method of detecting mutants or variants of SARS-CoV-2.

The term “polynucleotide”, as used herein, refers to a single or double stranded polymer composed of nucleotide monomers.

The term “nucleic acid”, as used herein, refers a polymer composed of nucleotides, e.g. deoxyribonucleotides or ribonucleotides.

The terms “ribonucleic acid” and “RNA”, as used herein, refers to a polymer composed of ribonucleotides.

Reference to the expression “5” end of a sequence segment refers to the localization of the sequence of nucleotides referred to is towards the 5′ terminal end of the sequence segment.

Reference to the expression “3′” end region of a sequence segment\” there is intended that the localization of the sequence of nucleotides referred to is towards the 3′ terminal end of the sequence segment.

The term “amplicon” as used herein refers to a polynucleotide DNA or RNA molecule that is the product of an enzymatic or chemical-based amplification event or reaction. An amplicon may be single or double stranded. Enzymatic or chemical-based amplification events or reactions include, without limitation, the polymerase chain reaction (PCR), loop mediated isothermal amplification, rolling circle amplification, nucleic acid sequence base amplification, and ligase chain reaction or recombinase polymerase amplification.

The term “primer” or “primer polynucleotide”, as used herein, refers to an oligonucleotide that can hybridize to a template nucleic acid and permit chain extension or elongation using a nucleotide incorporating biocatalyst. A primer nucleic acid that is at least partially complementary to a subsequence of a template nucleic acid is typically sufficient to hybridize with the template nucleic acid for extension to occur. primer nucleic acid can be labeled, if desired, by incorporating a label detectable by radiological, spectroscopic, photochemical, biochemical, immunochemical, or chemical techniques.

The term, “extended primer”, as used herein, refers to a primer to which one or more additional nucleotides have been added. “Primer extension” is the action of the enzyme by which additional nucleotides are added to the primer.

The term “complementary”, as used herein, refers to the topological compatibility or matching together of interacting surfaces of a probe molecule, such as a primer, and its target. Thus, the target and its probe can be described as complementary, and furthermore, the contact surface characteristics are complementary to each other.

The term “hybridization” refers to a process of establishing a non-covalent, sequence-specific interaction between two or more complementary strands of nucleic acids into a single hybrid, which in the case of two strands is referred to as a duplex.

The term “anneal” refers to the process by which a single-stranded nucleic acid sequence pairs by hydrogen bonds to a complementary sequence, forming a double-stranded nucleic acid sequence, including the reformation (renaturation) of complementary strands that were separated by heat (thermally denatured)

The term “subject”, as used herein, refers is to an individual. Non-limiting examples of a subject may include, for example, domesticated animals, such as cats, dogs, etc., livestock (e.g., cattle, horses, pigs, sheep, goats, etc.), laboratory animals (e.g., mouse, rabbit, rat, guinea pig, etc.) mammals, non-human mammals, primates, non-human primates, rodents, birds, reptiles, amphibians, fish, and any other animal. The subject may be a mammal such as a primate or a human.

In one example, an “adaptor” is a single stranded DNA. The adaptors are versatile as their sequence and length can be changed for various applications (LAMP, qPCR, bioanalyzer . . . ) and can have moieties attached to their 3′ and 5′ ends for other detection modalities (DTECT-Fluo).

The term “detectable label”, as used herein, refers to a composition that when linked to a molecule of interest renders the latter detectable, via, for example, spectroscopic, photochemical, biochemical, immunochemical, or chemical means. For example, useful labels may include radioactive isotopes, magnetic beads, metallic beads, colloidal particles, fluorescent dyes, electron-dense reagents, enzymes (for example, as commonly used in an ELISA), biotin, digoxigenin, or haptens.

The term “next generation sequencing” (NGS) includes any form of high-throughput DNA or RNA sequencing. This includes, without limitation, sequencing by synthesis, sequencing by ligation, nanopore sequencing, single-molecule real-time sequencing and ion semiconductor sequencing.

In some aspects, there is provided a method of detecting a mutation in a target polynucleotide in a sample from a subject.

Accordingly, the methods herein may be used in the detection or identification of such polynucleotide mutations which may be indicate the presence or absence of a particular mutation, sequence variation, or polymorphism.

Polymorphisms include both naturally occurring, somatic sequence variations and those arising from mutation.

In some examples, there is provided methods for the identification of mutations in a target polynucleotide for identifying mutations associated with disease and/or markers thereof.

In some examples, there is provided methods for the identification of mutations in a target polynucleotide in microorganism(s), including but not limited to, bacteria, fungi, protozoa, ciliates, and viruses. The microorganisms are not limited to a particular genus, species, strain, or serotype.

In some examples, there is provided methods for the identification of a mutation in a target polynucleotide from a sample for rapid and accurate identification of sequence variations that are genetic markers of disease, which can be used to diagnose or determine the prognosis of a disease.

The identification of these “disease” markers is dependent on the ability to detect changes in genomic markers in order to identify errant genes or polymorphisms. Genomic markers (all genetic loci including single nucleotide polymorphisms (SNPs), microsatellites and other noncoding genomic regions, tandem repeats, introns and exons) can be used for the identification of all organisms, including humans. These markers provide a way to not only identify populations but also allow stratification of populations according to their response to disease, drug treatment, resistance to environmental agents, and other factors.

Diseases characterized by genetic markers can include, but are not limited to, atherosclerosis, obesity, diabetes, autoimmune disorders, and cancer.

The term “cancer”, as used herein, refers to a variety of conditions caused by the abnormal, uncontrolled growth of cells. Cells capable of causing cancer, referred to as “cancer cells”, possess characteristic properties such as uncontrolled proliferation, immortality, metastatic potential, rapid growth and proliferation rate, and/or certain typical morphological features. Cancer cells may be in the form of a tumour, but such cells may also exist alone within a subject, or may be a non-tumorigenic cancer cell. A cancer can be detected in any of a number of ways, including, but not limited to, detecting the presence of a tumor or tumors (e.g., by clinical or radiological means), examining cells within a tumor or from another biological sample (e.g., from a tissue biopsy), measuring blood markers indicative of cancer, and detecting a genotype indicative of a cancer. However, a negative result in one or more of the above detection methods does not necessarily indicate the absence of cancer, e.g., a patient who has exhibited a complete response to a cancer treatment may still have a cancer, as evidenced by a subsequent relapse.

Accordingly, the identification of mutations in a target polynucleotide in a sample from a subject may be used in applications, including but not limited to, oncology diagnostics, animal breeding, precision genetic editing applications,—including but not limited to base editing, prime editing, CRISPR, in laboratory animal models or plants/crops.

As used herein, “sample” or “biological sample” refers to a composition containing a material to be detected, such as a target polynucleotide.

In some examples, “sample” or “biological sample” refers to materials obtained from or derived from a subject or patient. A sample or biological sample includes sections of tissues such as biopsy (e.g., tumor biopsy) and autopsy samples, and frozen sections taken for histological purposes. Such samples include bodily fluids such as blood and blood fractions or products (e.g., serum, plasma, platelets, red blood cells, circulating tumor cells, and the like), lymph, sputum, tissue, cultured cells (e.g., primary cultures, explants, and transformed cells) stool, urine, synovial fluid, joint tissue, synovial tissue, synoviocytes, fibroblast-like synoviocytes, macrophage-like synoviocytes, immune cells, hematopoietic cells, fibroblasts, macrophages, T cells, etc.

In some examples, a biological sample may be from a sample from a nasal swab, a sample from an oropharyngeal swab, a sputum sample, a lower respiratory tract aspirate, a bronchoalveolar lavage, a nasopharyngeal wash/aspirate or a nasal aspirate.

In other examples, “sample” or “biological” sample may refer to any material obtained from, for example, an animal such as a human or other mammal, a plant, a bacterium, a fungus, a protist or a virus.

Methods for obtaining a sample or biological sample are known.

In some examples, the sample is from a eukaryote, a prokaryote, or a viruses.

In some examples, the sample is from a mammal, a plant, a bacterium, a fungus, a protest, or a virus.

In a specific example, the subject is a human.

In one aspect there is provided a method of detecting a mutation in a target sequence of a severe acute respiratory syndrome coronavirus 2 (SARS-CoV-2) polynucleotide sample, the method comprising:
(a) contacting a SARS-CoV-2 polynucleotide containing sample with at least one primer polynucleotide comprising, or consisting of, a structure of formula (I):

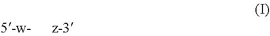

wherein w comprises or consists of an Acu1-tagging primer polynucleotide, and z comprises or consists of a target sequence polynucleotide, wherein w comprises or consists of a structure of formula (II).

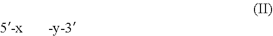

wherein x comprises or consists of an Acu1-handle polynucleotide and y comprises or consists of an Acu1 motif polynucleotide, under condition to generate an Acu1-tagged amplicon;
(b) contacting the Acu1-tagged amplicon with Acu1, one or more variant adaptors at a concentration of about 250 uM, and a ligase, to generate a reaction mixture, and
(c) subjecting the reaction mixture to a reaction time and reaction temperature, to generate a ligation product.

In one example, the ligase is a T4 ligase.

In one example, the T4 ligase is a heat resistant (Hi-T4) T4 ligase, a salt-tolerant (Salt-T4) T4 ligase or, a highly concentrated (T4-HC) T4 ligase.

In one example, the reaction temperature is between about 16° C. and about 37° C.

In one example, the reaction temperature is between about room temperature.

In one example, the reaction time is about 10 min or less than 10 min.

In one aspect there is provided a method of detecting a mutation in a target polynucleotide in a sample from a subject, the method comprising:
(a) contacting a polynucleotide containing sample with at least one primer polynucleotide comprising, or consisting of, a structure of formula (I):

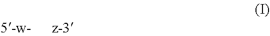

wherein w comprises or consists of an Acu1-tagging primer polynucleotide, and z comprises or consists of a target sequence polynucleotide,
wherein w comprises or consists of a structure of formula (II).

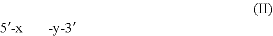

wherein x comprises or consists of an Acu1-handle polynucleotide and y comprises or consists of an Acu1 motif polynucleotide,
under condition to generate an Acu1-tagged amplicon;
(b) contacting the Acu1-tagged amplicon with Acu1, one or more variant adaptors at a concentration of about 250 uM, and a ligase, to generate a reaction mixture, and
(c) subjecting the reaction mixture to a reaction time and reaction temperature, to generate a ligation product.

In one example, step b) further comprises a competitor DNA.

In one example, the concentration of the competitor DNA is about 1 pmol.

In one example, the ligase is a T4 ligase.

In one example, the T4 ligase is a heat resistant (Hi-T4) T4 ligase, a salt-tolerant (Salt-T4) T4 ligase or, a highly concentrated (T4-HC) T4 ligase.

In one example, the reaction temperature is between about 16° C. and about 37° C.

In one example, the reaction temperature is about room temperature.

In one example, the reaction time is about 10 min or less than 10 min.

In one example, the sample is from a eukaryote, a prokaryote, or a virus.

In one example, the subject is a mammal, a plant, a bacterium, a fungus, a protest, or a virus.

In one example, the sample is isolated from a cell, a cell pellet, a cell extract, a tissue, a biopsy, or biological fluid, obtained from the subject

In one example, the target polynucleotide is the PIK3R1 gene.

In one example, the sample is from a cancer sample.

In one example, the sample is from a nasal swab, a sample from an oropharyngeal swab, a sputum sample, a lower respiratory tract aspirate, a bronchoalveolar lavage, a nasopharyngeal wash/aspirate or a nasal aspirate.

In one example, the subject is a human.

The term “about”, as used herein, when referring to a measurable value such as an amount, a percentage, and the like, is meant to encompass variations of ±20%, ±10%, ±5%, or ±1% from the measurable value.

Method of the invention are conveniently practiced by providing the compounds and/or compositions used in such method in the form of a kit. Such kit preferably contains the composition. Such a kit preferably contains instructions for the use thereof.

To gain a better understanding of the invention described herein, the following examples are set forth. It should be understood that these examples are for illustrative purposes only. Therefore, they should not limit the scope of this invention in anyway.

### EXAMPLES

### Example 1

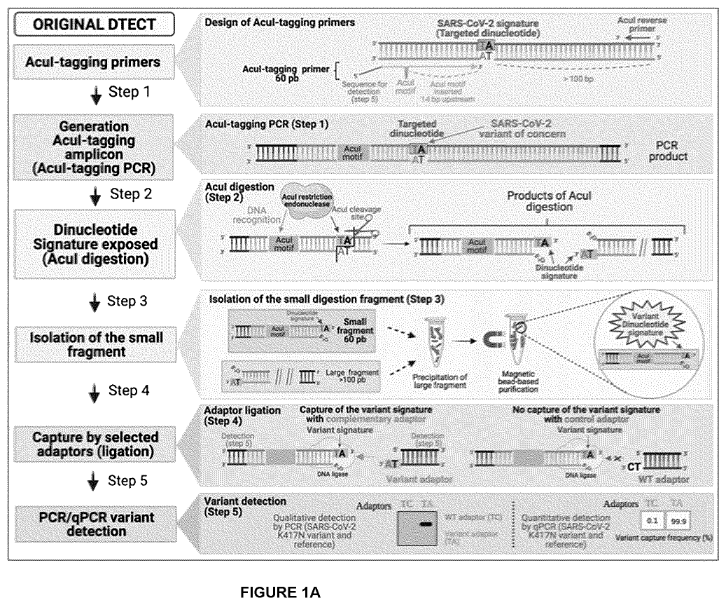

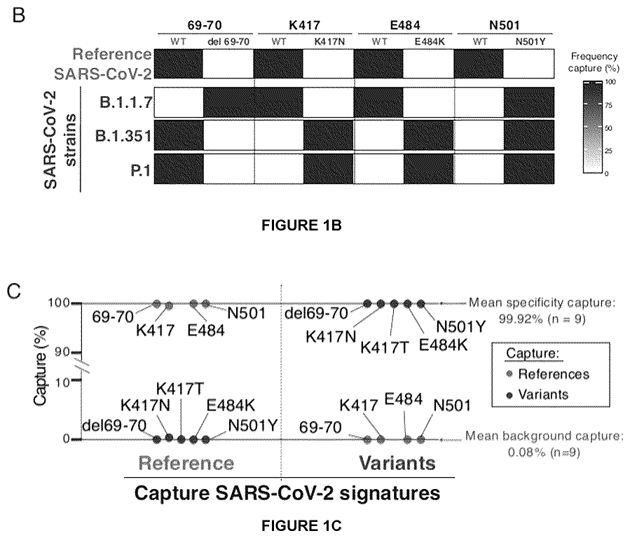

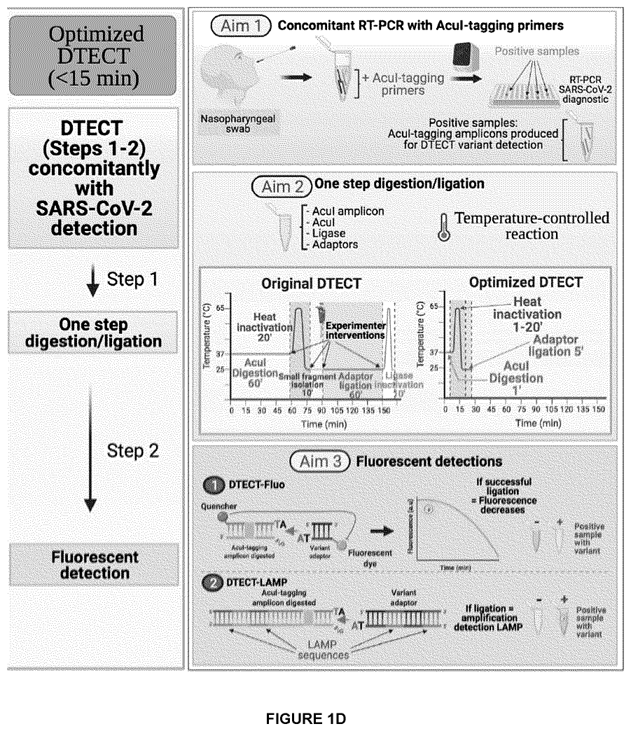

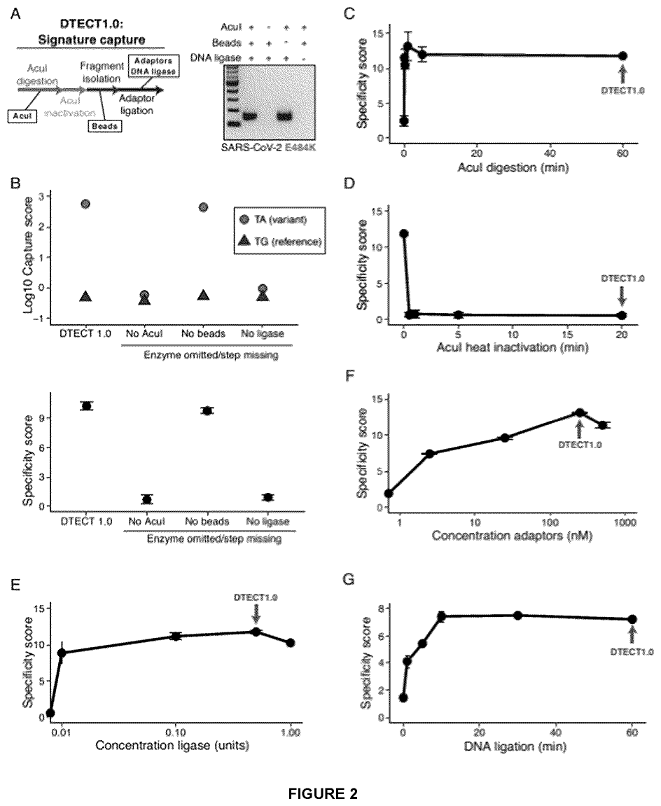

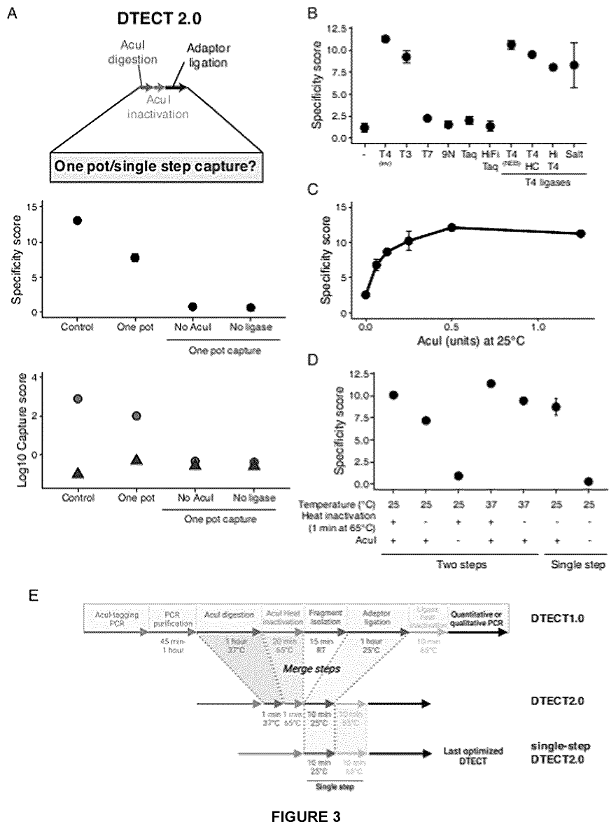

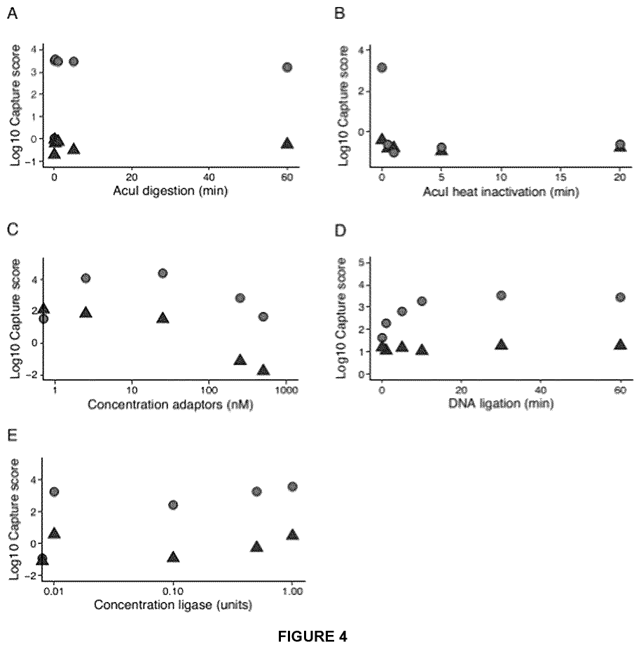

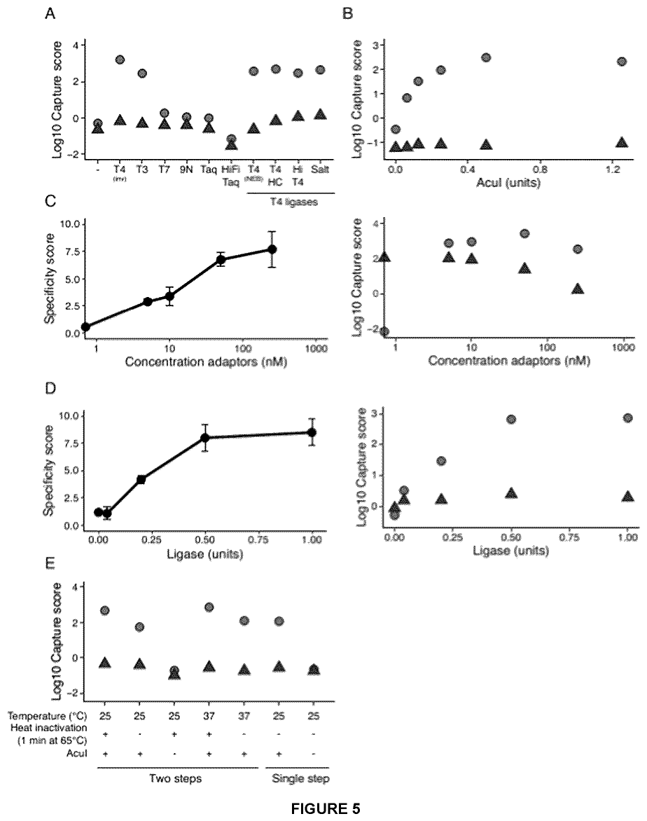

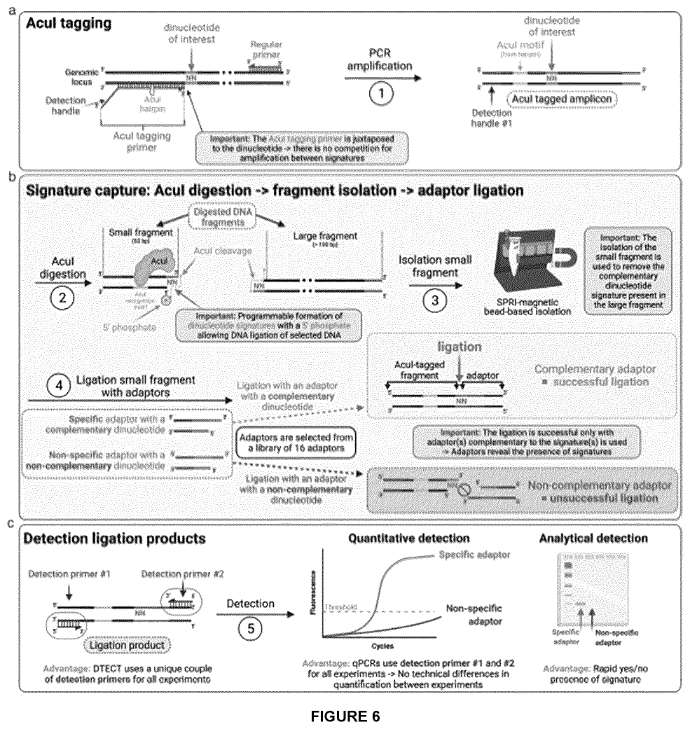

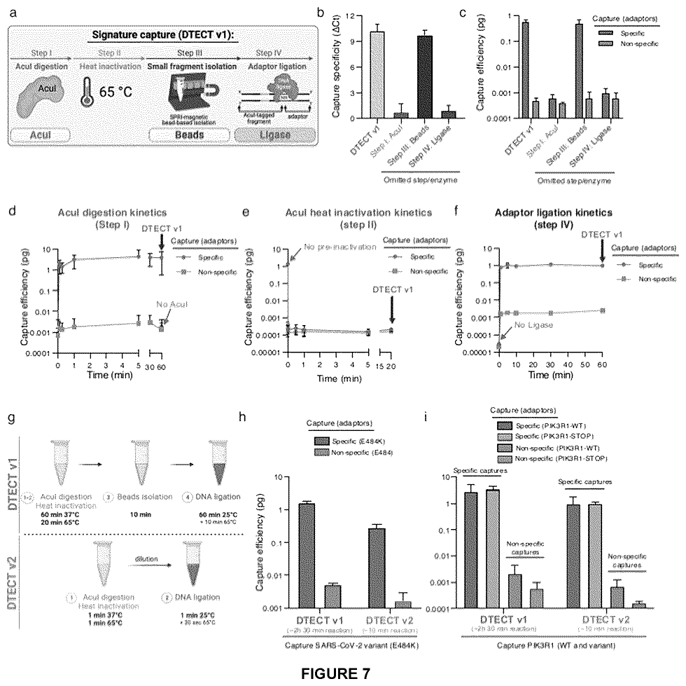

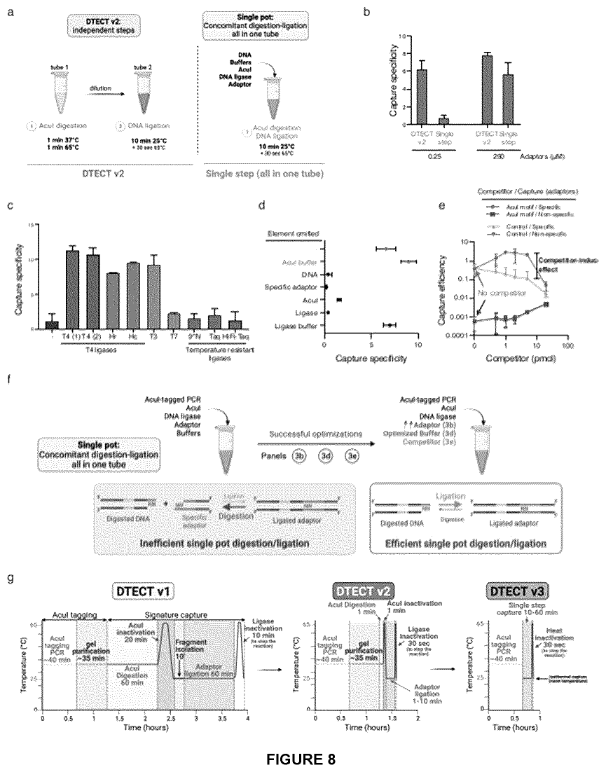

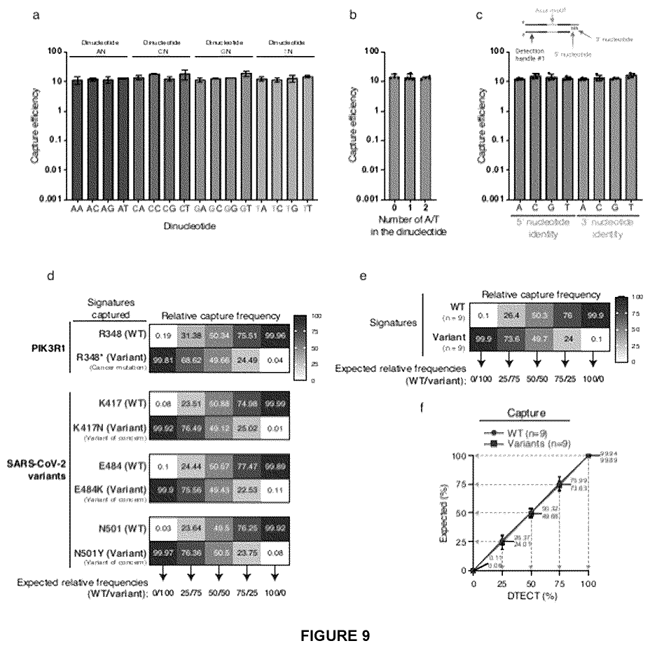

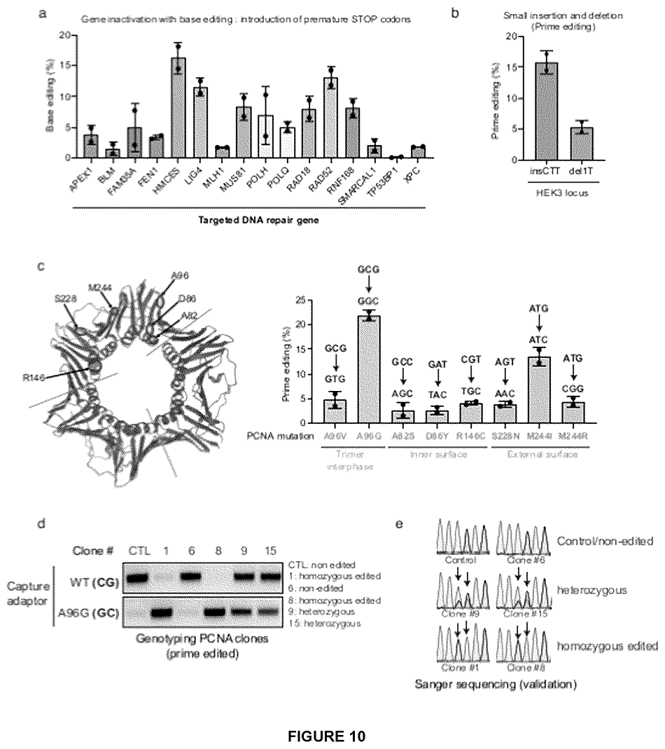

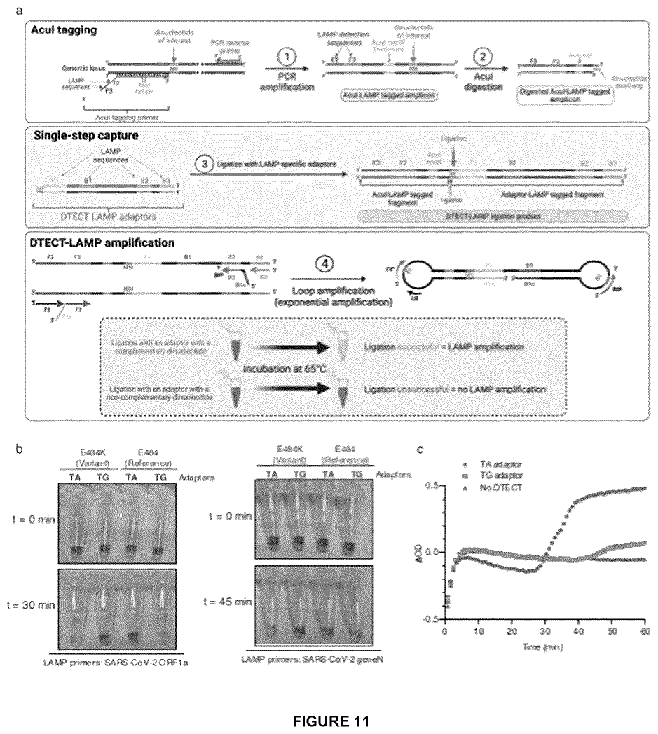

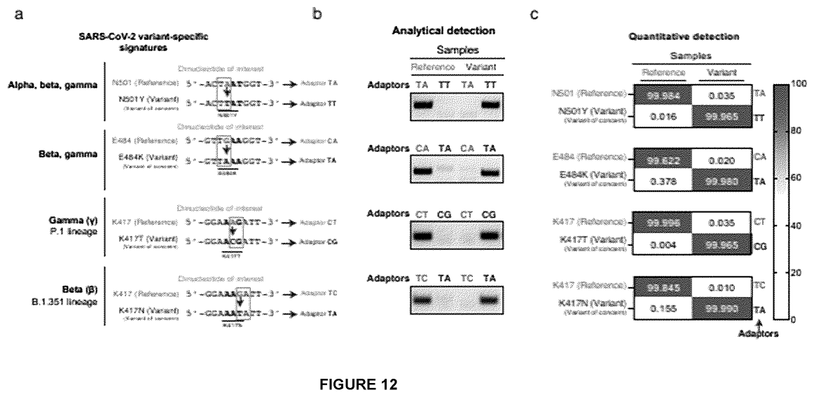

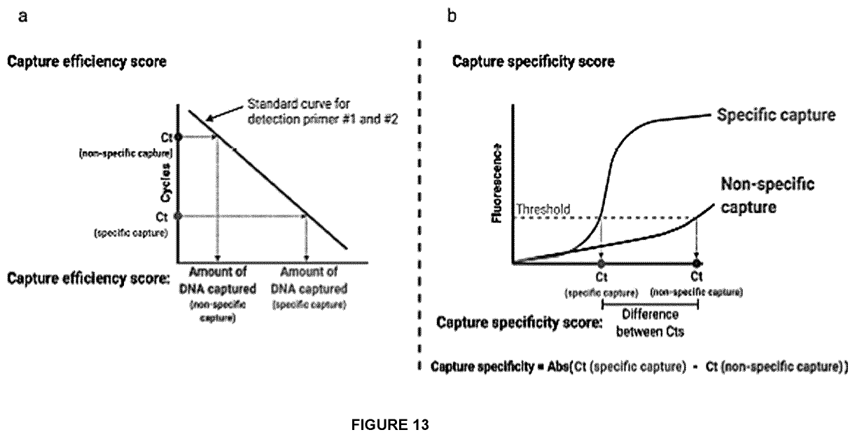

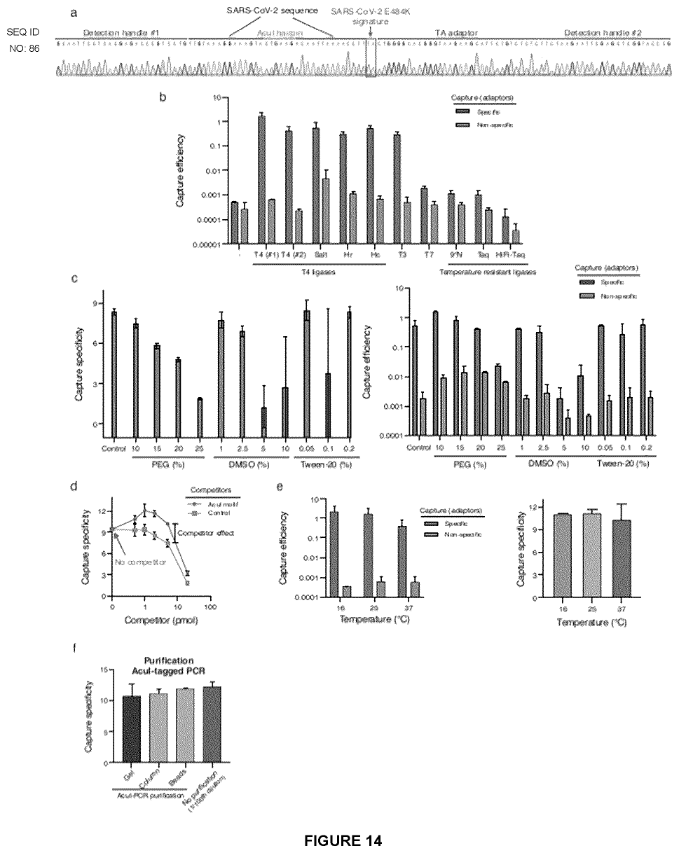

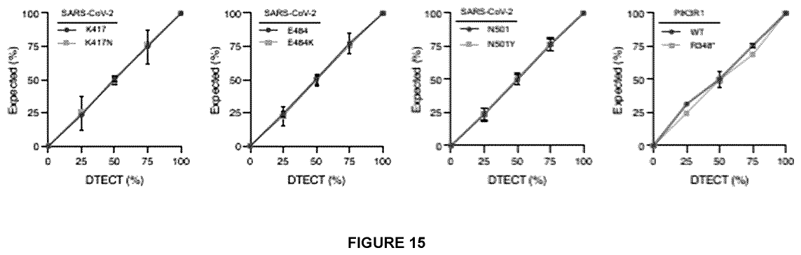

We have previously established Dinucleotide signaTurE CapTure (DTECT) (FIG. 1A). This multi-step approach that enables sequencing-free capture-based detection of pathogenic variants in human disorders or introduced by genome editing technologies³³, such as modern CRISPR-based technologies³⁴⁻³⁷. First, a PCR amplicon introduces an Acu1 motif 14 nt upstream of a dinucleotide signature of interest (FIG. 1A, step 1). The Acu1-tagging primer is juxtaposed to the signature, so the reference and variant signatures do not compete for amplification, like in allele-specific PCR approaches. Next, Acu1 digests the amplicon to create a 3′ dinucleotide overhang, exposing the dinucleotide signature (FIG. 1A, step 2). Then, the small digestion fragment is isolated, using Solid Phase Reversible Immobilization (SPRI) beads, to decrease potential interference between the digestion fragments and adaptors during ligation (FIG. 1A, step 3). Next, DNA ligation ligates a selected complementary (or non-complementary) adaptor to the digested amplicon (FIG. 1A, step 4). The use of non-complementary adaptors validates the specificity of the detection (e.g., FIG. 2a). Analysis of the captured material integrates both quantitative (FIG. 1A, step 5 right) and qualitative detection (FIG. 1A, step 5 left) to either provide a quantitative assessment of the different population of variants or a rapid determination of the presence/absence of variants.

Here, we demonstrate that DTECT captures SARS-CoV-2 variant signatures from different strains with high efficiency and specificity.

We describe herein an enhanced version of DTECT that we validate on emerging SARS-CoV-2 variants of concern (VOC). The improvements described herein limit the number of steps, hands-on time to derive a 10 min single-step one-pot signature capture. These experiments allow direct implementation of DTECT into standard SARS-CoV-2 diagnostics to detect variants of concern and expand the possibilities to apply DTECT for basic laboratory experiments, such as CRISPR-based precision genome editing.

### Results

DTECT relies on two successive enzymatic reactions 1) digestion using a type IIS restriction endonuclease to expose genetic signatures and 2) ligation of DNA adaptors complementary to the signatures using a DNA ligase to capture signatures (FIG. 1A). This approach requires an Acu1-tagged amplicon generated from nucleic acid samples (e.g., reverse-transcribed RNA or genomic DNA). Then, analytical or quantitative PCR is used to qualitatively or quantitatively measure the ligated product, which directly correlates with the presence of the signature in the nucleic acid sample (FIG. 1A).

DTECT Captures SARS-CoV-2 Variant Signatures with High Specificity

Given the high versatility of DTECT to identify all types of dinucleotide signatures, we tested whether DTECT can capture SARS-CoV-2 genetic signatures. Emerging circulating strains of SARS-CoV-2 (e.g., B.1.17, B.1.351, and P.1 lineages) carry multiple genomic mutations of concern which increase transmissibility and partially prevent recognition by antibodies (e.g., del69-70, K417N, K417T, E484K, and N501Y). We designed Acu1-tagging primers to capture various SARS-CoV-2 signatures from the SARS-CoV-2 reference sequence³⁸. Using synthetic DNA molecules that encode various mutations, we determined that DTECT captures each signature of the SARS-CoV-2 reference sequence with high sensitivity and specificity, as the emerging variants were not detected (FIG. 1B). Moreover, DTECT identifies each variant signature, including a small deletion and substitution mutations with adaptors specifically recognizing variant signatures, but not the original reference signatures (FIGS. 1B and 1C). Interestingly, the capture specificity is high as the non-specific background is low (0.08% n=9) (FIG. 1C). These data demonstrate that DTECT performs well at identifying SARS-CoV-2 signatures and distinguishes between the different strains with high specificity.

DTECT required about 4-5 hours to execute and requires multi-step procedures, which is not optimal for routine variant detection. In addition, they do not facilitate the execution of basic laboratory experiments.

Therefore, we evaluated DTECT for its performance on ligation efficiency (referred to as capture score) and specificity (specificity score).

### Elimination of Bead Isolation

DTECT utilizes two sequential enzymatic reactions to capture specific signatures. First, the type IIS restriction enzyme Acu1 digests a genomic amplicon to generate a 3′ dinucleotide overhang. Second, a DNA ligase ligates specific DNA adaptors complementary to either the reference or variant signatures. Finally, an isolation step that separates the two enzymatic activities serves to isolate one of the two DNA fragments. This step helps to preferentially ligate the adaptor without reassembling the two DNA fragments generated at the digestion step, thereby enabling high precision ligation.

To confirm that these three steps (Acu1 digestion, beads isolation, and adaptor ligation) are critical to DTECT, we tested the original DTECT (also referred to as DTECT1.0) with or without Acu1, the isolation beads step, or DNA ligase (FIG. 2A). DTECT1.0 captured the SARS-CoV-2 E484K variant with high efficiency and specificity (FIG. 2B). In addition, the omission of Acu1 or ligase abolishes signature capture to the same level as a non-specific adaptor which recognizes the E484 SARS-CoV-2 reference signature (FIG. 2B). These data show that Acu1 activity exposed the overhang signature and that the DNA ligase activity is required to achieve signature capture.

Surprisingly, omitting the beads isolation step affected neither capture efficiency nor specificity (FIG. 2B) of the SARS-CoV-2 variant capture.

An analysis of the product of ligation by analytic PCR confirmed the expected product (FIG. 2A).

These results indicate that the beads isolation step is dispensable, which not only decreases the overall cost of DTECT but also facilitates the implementation of an improved method with the minimal requirement for off-the-shelves enzymes.

Altogether, these results demonstrate that the sensitivity and specificity of DTECT1.0 can be improved.

### Development of an Accelerated and Simplified DTECT Assay

These data prompted us to reassess each condition of DTECT1.0 to develop an optimized and accelerated signature capture with enhanced capture efficiency and specificity.

In the DTECT1.0 protocol, 1.25 units of Acu1 digest 0.2 pmol of Acu1-tagged amplicon during 60 minutes at 37° C.

We conducted a time-course experiment ranging between 10 seconds and 60 minutes to determine the optimal digestion conditions.

Surprisingly, we observed that the digestion of 0.2 pmol of DNA with 1.25 units of Acu1 is rapid. In some examples, the digestion is considered to be substantially instantaneous (FIG. 2C) In some examples, digestion occurred within 15 seconds. In some examples, digestion occurred in less than 15 seconds.

A 10-second incubation leads to complete capture, comparable to a 60 min digestion (FIG. 2C and FIG. 4A, green circles). As a control, a reaction lacking Acu1 and incubated for 60 min led to a background capture (FIG. 4A—the green circle at t=0 min) comparable to the capture using a non-specific adaptor (FIG. 4A—red triangles). Notably, 10-second incubation also does not affect capture specificity as indicated by the comparable specificity to the 60 min reference used in DTECT1.0 (FIG. 4A—red triangle at 60 min).

Inactivation of Acu1 by incubating Acu1 at 65° C. for 20 min, is suggested by the suppliers to limit potential interference between Acu1 and ligation.

To test whether Acu1 heat inactivation can be accelerated, we conducted a time-course experiment (FIG. 2D). We heat-inactivated Acu1 at 65° C. for a different duration, ranging from 30 seconds to 20 min. Then after heat inactivation, the Acu1-tagged amplicon is added to induce digestion only if a residual activity is present. We restricted the digestion to 1 min at 37° C. because previous experiments revealed that digestion is rapid (FIG. 2C). The no heat inactivation control led to a robust capture, which confirmed the rapid digestion (FIG. 2D and FIG. 4B). Interestingly, incubation of Acu1 for 30 seconds at 65° C. completely prevents capture at the same level as 20 min (FIG. 2D and FIG. 4A), thereby completely blocking digestion due to Acu1 denaturation in under 30 seconds. These data demonstrate that Acu1 digestion and heat inactivation are instantaneous.

Next, we decided to test how the ligation reaction can be optimized to improve capture efficiency and specificity. The quantity of substrates (adaptors) and enzyme concentration (DNA ligase) influence DNA ligation efficiency. Therefore, we titrated the concentration of DNA ligase (FIG. 2E and FIG. 4E) and adaptors (FIG. 2F and FIG. 4C) and determined the condition that provides the highest capture efficiency and specificity. Importantly, these experiments also reveal that the efficiency (FIG. 2E) and specificity (FIG. 4E) of the capture are not affected by a 1/100^{th} dilution of the ligase, suggesting that a minimal quantity of ligase is required. Finally, we tested how the capture efficiency is affected by incubation time. We found that a 10-minute incubation is sufficient to obtain complete ligation, compared to the 1 hour in DTECT1.0 without affecting specificity (FIG. 2G and FIG. 4D). These results reveal that the ligation step can be limited to 10 minutes.

Altogether, we determined the optimal conditions for high efficiency and specificity capture. These changes include removing the beads isolation step and implementing rapid digestion and ligation reactions. These enhancements result in a 92% decrease in the duration of the capture, unlocking a rapid (12 min) and sensitive signature capture, referred to as DTECT2.0 (FIG. 2G). Strikingly, these results also suggest that we may merge the two enzymatic activities (Acu1 digestion and DNA ligation) into a single pot in an optimized buffer that can accommodate both activities.

### Development of an Isothermal Single-Step One-Pot Capture

To test the compatibility of Acu1 and ligase, we combined in a single pot but two independent steps: digestion and ligation reactions. First, Acu1 was added with the DNA and incubated for 2 min (1 min digestion at 37° C. and 1 min denaturation at 65° C.). Then, DNA ligase and adaptors were added for 10 min at 25° C. Ligation reactions were immediately stopped by heating the reaction for 10 min to ensure the quantification of the capture is not affected by subsequent handling of the samples. Remarkably, this single pot experiment led to a robust and specific capture (FIG. 3A). The same experiment without Acu1 or ligase led to a capture efficiency comparable to an unspecific adaptor demonstrating the specificity of the signature capture in a single pot (FIG. 3A). Next, we systematically evaluated the activity of multiple DNA ligases, such as T3, T7, 9°N and Taq ligases, to determine the most effective ligase in a single pot. We utilized optimal ligation conditions for each ligase. Among the different ligases, the T4 ligase showed the most robust capture activity followed by the T3 (FIG. 3B), consistent with their preference for cohesive ends. In contrast, T7, 9°N, and Taq ligases did not robustly capture the signature (FIG. 3B) as they prefer to ligate nicks or adjacent DNA strands. The high performance of the T4 ligase prompted us to test multiple engineered T4 ligases, such as heat resistant (Hi-T4), salt-tolerant (Salt-T4), highly concentrated (T4-HC) T4 ligases, and also a T4 ligase from a different supplier. Each T4 ligase performed well at capturing the dinucleotide signature with high specificity (FIG. 3B). Notably, the one-pot capture using the regular T4 ligase from two different suppliers lead to the same robust and specific capture (FIG. 3B and Supplementary FIG. 2A), confirming the robustness of the single pot digestion-ligation signature capture.

Our objective is to induce the capture in a single-step in which Acu1 and T4 ligase activities function simultaneously at room temperature. This approach can only be successful if minimal Acu1 activity is provided to limit further digestion of the adaptor ligation reaction. We titrated the amount of Acu1 and measured its activity at 25° C. Interestingly, we find that a dilution of Acu1 to 0.5 units leads to optimal capture, comparable with the reference (FIG. 3C and FIG. 5B). These data show that the amount of Acu1 can be minimized without losing performance in the single pot DTECT assay. Finally, we also titrated the quantity of adaptors (FIG. 5C) and ligases (FIG. 5D) to determine the optimal concentration in the single pot reaction. Altogether, these experiments help determine the conditions that provide a single-step isothermal digestion-ligation with minimal ligation activity lost due to Acu1.

With the demonstration that digestion and ligation can be incubated in a single tube in optimized conditions, we tested whether the single pot isothermal incubation of the digestion and ligation for 10 min at room temperature. Strikingly, we found that signature capture is efficient even without Acu1 inactivation, demonstrating that the ligation is not affected by the presence of minimal Acu1 activity (FIG. 3D and FIG. 5E). While not wishing to be bound by theory, given that the recognition motif of Acu1 and its cleavage location are far away, we speculate that Acu1 probably remains bound to its substrate for an extended period limiting the possibility for enzyme turnover, thereby facilitating the ligation of the adaptors. As controls, omitting Acu1 in the reaction did not capture confirming the dependency for Acu1 activity (FIG. 3D and FIG. 5E). These data reveal a highly efficient and specific single-step capture at room temperature with minimal enzymes without requiring experimenter intervention.

Finally, we tested whether direct dilution of the Acu1-tagged amplicon or various purification protocols, including gel purification, column purification, or beads purification are compatible with the single-step digestion/ligation. We observed that the purification of Acu1-tagged amplicon is dispensable as purification or dilution of the PCR leads to comparable results (data not shown). Notably, a large window of dilution enables high-efficiency capture without losing specificity. This data suggests that the Acu1-tagged amplicon does not require purification but also that the performance of DTECT2.0 will not be affected by the PCR efficiency or the quantity of starting material. These data establish an improved method for the highly sensitive single-step capture of genomic signatures.

### Discussion

In this work, we developed a new capture of genomic signatures for the straightforward detection of SARS-CoV-2 variants. The development of a single-step single-pot DTECT is particularly useful for precision genome editing applications because it will help democratize DTECT for basic research labs.

Alternative detection methods include sequencing technologies, such as next-generation sequencing or Sanger sequencing. However, these approaches are expensive, have a considerable turnaround time of several days (Sanger sequencing) to weeks (NGS) (compared to a few hours for DTECT), and require the involvement of third parties. On the other hand, DTECT2.0 as described herein is accessible because it only requires off-the-shelf reagents (e.g., T4 ligase and Acu1), which are available from various suppliers, and minimal equipment (e.g., thermocycler and qPCR). In addition, an advantage of DTECT2.0 is that it uses a standard library of 16 adaptors to detect each possible dinucleotide signature. Thus, DTECT2.0 offers significant advantages over approaches utilizing sequencing technologies for the rapid monitoring of variants. For instance, DTECT2.0 identifies all variant types by capturing targeted signatures with a unique library of adaptors and achieves high specificity and sensitivity detection of molecular signatures through a strong covalent ligation (i.e., capture). Moreover, multiple analysis modalities can be derived to analyze the ligated product(s) as a signal for the presence of variants in patient specimens.

DTECT2.0 is a robust molecular diagnostic tool with several significant features that makes it more reliable, specific, and efficient than other rapid diagnostic tests that utilize mutation-specific PCR primers and probes to identify variants²⁶⁻²⁸ Indeed, these methods have a low specificity conferred by a single nucleotide mismatch to differentiate a variant from the reference (e.g., a 25 nt probe/primer: 1/25 nt→4% specificity target). In contrast, DTECT relies on a dinucleotide capture to differentiate the variant from the reference (½ nt→50% difference in the target), resulting in a strong specificity. Additionally, DTECT is a ligation-based approach that generates covalent phosphodiester bonds between signatures and adaptors, creating stable ligation products, unlike primers/probes approaches which rely on weak and transient nucleic acid interactions. The production of a stable ligated product allows the deployment of multiple modalities to analyze the captured material, as proposed below. Thus, DTECT is also particularly relevant for clinical applications. Indeed, DTECT provides robust internal controls in all SARS-CoV-2 positive samples because it must always detect either the WT or the variant SARS-CoV-2 signatures. Moreover, each variant can be detected using four independent signatures (2 flanking Acu1-tagging primers from each DNA strand), providing rigorous validations required to deliver high-confidence clinical results. Finally, DTECT is a robust qualitative and quantitative approach with limited technical variabilities because it exploits a unique couple of qPCR oligo pair to analyze the ligation products (FIG. 1A, Step 5). In contrast, other approaches require a unique design and testing of multiple variant-specific probes and oligos for each variant. The ease to capture desired nucleic acid signatures with standard adaptors and unique qPCR oligo pair will prove beneficial for immediate mobilization of DTECT against future emerging variants without requiring additional optimizations or changes in the DTECT protocol.

An appealing advantage of DTECT for further improvements is the flexibility of the adaptors and 5′-end of the Acu1-tagging oligos. For instance, 5′- and 3′-ends of the adaptors are available for the addition of dyes/quenchers, and the modifications in the DNA sequences do not affect DTECT efficiency.

Loop-mediated isothermal amplification (LAMP) is a sequence-specific isothermal DNA amplification method that produces a large quantity of DNA³⁹. The rapid production of DNA modifies the pH, which induces a change in the color of pH-sensitive dyes⁴⁰ that can be visualized by the naked eye or under blue/UV light. We will couple DTECT with LAMP by integrating the LAMP-specific sequences into the adaptors and 5′ sequence of the Acu1-tagging primers. Therefore, upon ligation of the signatures to the adaptors, the LAMP sequences will be reconnected, generating an amplification signal that can be visualized in real-time. To optimize DTECT-LAMP, multiple color dyes such as calcein, hydroxynaphthol blue, SYBR green I, berberine and EvaGreen^{40,41} may be used By coupling our optimized single-step DTECT (<10 min) with LAMP (˜15-30 min), we expect to achieve visual detection of SARS-CoV-2 variants in <1 hr without instrumentation.

In another example, a quencher may be added (e.g., Iowa BlackFQ) and a fluorescent dye (e.g., 6-carboxyfluorescein) to the 5′- and 3′-ends of the Acu1-tagging oligo and adaptors. Various commercially available quenchers and dyes may either be placed at 5′- or 3′-end of the Acu1-tagging oligo and adaptors to determine the best combination for efficient and multiplexed signal detection. Upon successful covalent linkage induced by ligation of the adaptors to the complementary signature, the quencher will block fluorescence emission, resulting in a loss of fluorescence over time, as easily detectable with a transilluminator or a fluorescence plate reader⁴². Multiple adaptors with different dyes may be used to recognize various variant signatures will unlock multiplexed detection of variants. DTECT-Fluo will provide an all-in-one multiplexed detection of variants, in which all components are present (digestion, ligation, and detection) for real-time detection (<5 min total) without experimenter intervention.

### Material and Methods

### Synthetic DNA and Molecular Cloning

Synthetic DNA molecules containing portions of the SARS-CoV-2 genome with or without mutations were purchased as gBLOCK DNA fragments (IDT). The DNA fragments were resuspended in TE buffer, cloned into the pCR-Blunt II-TOPO vector (ThermoFisher Scientific), and transformed into DH5a. Successful cloning and SARS-CoV-2 sequence were confirmed by Sanger sequencing.

### Preparation of the Library of Adaptors

The same library of adaptors is used for the capture of 16 dinucleotide signatures. The library comprises 16 double-stranded DNA adaptors generated from 17 individual oligonucleotides (sequences available in table 1). It contains one constant oligonucleotide (named OB1), which contains a sequence at the 3′ end (5′-gaattcgagctcggtacccg-3′)(SEQ ID NO: 86) for the detection of the ligated products, and 16 individual oligonucleotides, which are composed of a sequence complementary to the constant oligonucleotide and one of the 16 different dinucleotides at their 3′ end (named OB2-OB17).

Each oligonucleotide is resuspended at a concentration of 100 μM in TE (10 mM Tris and 0.5 mM EDTA). The annealing reactions are composed of 2.5 μl of the constant oligonucleotide, 2.5 μl of each unique dinucleotide oligonucleotide, and 1× ligase buffer. The reactions are incubated for 5 min at 95° C. to remove any potential secondary structures followed by a gradual temperature decrease from 95° C. to 15° C. at a ramp rate of 1° C./s. Then, 100 μl H₂O is added to dilute the adaptors at 5 uM. Adaptors are stored at −20° C. or −80° C.

### Design of Acu1 Tagging Primers and PCR

The Acu1-tagging PCR utilizes a pair of primer named “Acu1-tagging primer” and “reverse primer”. The objective of the Acu1-tagging PCR is to insert an Acu1 motif 14 bp upstream from a targeted dinucleotide, introduce a handle that is used for the detection, and amplify the locus of interest.

The Acu1-tagging primers is a 60 nt long oligonucleotide that contains an Acu1 motif (5′-CTGAAG-3′) as a hairpin 14 np from the 3′ end of the primer. In addition, it also contains a non-complementary handle sequence of 25 nt (5′-GCAATTCCTCACGAGACCCGTCCTG-3′) (SEQ ID NO: 53) that is used for the detection. Therefore, the Acu1 tagging primer has the following architecture: 5′-N(15)CTGAAGN(14)-3′ (SEQ ID NO: 54) with “N” corresponding to A, T, G, or C bases complementary to the targeted locus.

The reverse primer is designed using Primer 3 (http://bioinfo.ut.ee/primer3-0.4.0/) with a length of “min=25, Opt=27, Max=30” and a Tm of “min=57.0° C., opt=60.0° C., max=63.0° C.”

The Acu1-tagging PCR is performed in a 25 μl with 1 unit Q5 polymerase as recommended (NEB), 1× Q5 buffer, 1 μM of each primer, 10 ng plasmid template, 0.1 mM dNTP in a thermocycler: 95° C. for 30 s; 40 cycles of 95° C. for 10 s, 58° C. for 10 s, 72° C. for 45s and a final amplification at 72° C. for 1 min. The PCR reaction is loaded on a 2% agarose gel in TAE buffer, and the amplicon is extracted from gel and column purified (Zymo Research #D4008). The purified Acu1-tagged amplicon is quantified with the nanodrop 2000 and stored at −20° C.

### DTECT1.0 Protocol

The original DTECT protocol has been conducted as detailed previously³³ Briefly, DTECT relies on the amplification of the genomic locus of interest using an Acu1-tagging primer. The purified Acu1 tagged amplicon is digested by Acu1 in a 20 μl reaction as follows: 0.2 pmol Acu1 tagged amplicon, 1.25 units Acu1 (NEB #0641) in 1× CutSmart buffer. The digestion is incubated at 37° C. for 1 hour followed by heat inactivation at 65° C. for 20 min. SPRI beads separate the digested fragments by mixing beads at a ratio of 1:1.8 of Agencourt AMPure XP magnetic beads. 10 μl of digestion is mixed with 18 μl of beads by pipetting up and down ten times and incubated at room temperature for 5 min. The tube is then placed on a magnetic rack, and the supernatant is recovered and diluted in 40 μl H₂O. Next, the ligation of the adaptors is performed in the following reaction: 6.5 μl H₂O, 2 μl of 5× ligase buffer, 0.5 μl T4 ligase (ThermoFisher Scientific), 0.5 μl adaptor, and 0.5 μl of the purified digested product. The ligation reaction is incubated for 1 hour at 25° C. in a thermocycler. The reaction was then stopped by incubating the reaction at 65° C. for 10 min to denature the ligase. The captured material was detected either using quantitative PCR or analytical PCR.

The qPCR is conducted using the QuantStudio 6 (Applied Biosystems). qPCR reactions were performed as follows: 5 μl of 2× SYBR Green master mix, 0.1 μl of primer OB1 (100 μM), 0.1 μl of primer OB2 (100 μM) and 1 μl of ligated products in a 10 μl reaction. The qPCR program is the following: 1) A hold stage of 1 cycle at 50.0° C. for 2 min and 95.0° C. for 10 min. 2) A PCR stage of 40 cycles at 95° C. for 10 seconds and 60° C. for 30 seconds. 3) A melt curve stage of 1 cycle of incubations at 95° C. for 15 seconds, 60° C. for 1 min, and 95° C. for 15 seconds. The quantification of the captured material (capture score) and the difference between the specific and non-specific adaptor (specificity score) are calculated as described below.

The analytical detection is performed by standard Q5 PCR in a 12.5 μl containing 0.1 μl Q5 polymerase, 1× Q5 buffer, 0.5 μM OB18, 0.5 μl OB19, 0.05 mM dNTP, and 1 μl ligation products. The PCR program (Proflex 3×32) for the analytical reaction is the following: 95° C. for 1 min and 22 cycles of 95° C. for 10 s, 65° C. for 5 s and 72° C. for 7 s. The PCR reaction was incubated with SYBR Gold (ThermoFisher Scientific), loading dye, and loaded on a 2% agarose gel with TAE buffer.

### DTECT Optimizations

The experiment without bead isolation was carried out following the DTECT1.0 procedure, but the bead step was omitted. Without the beads step, the digestion reaction was diluted by adding 100 μl of H₂O. This dilution was subsequently used in regular ligation. In addition, enzymes have been diluted in their working buffer, such that Acu1 was diluted in 1× Cutsmart buffer and T4 ligase was diluted in 1× ligase buffer. All reactions were conducted in independent duplicates. All incubations were conducted in a thermocycler.

### DTECT2.0 Protocol

The DTECT2.0 protocol relies on DTECT1.0 but includes several optimizations. For example, the duration of the digestion/inactivation has been shortened, a dilution in H₂O has replaced the bead isolation step, and the adaptor ligation step has been shortened.

The Acu1-tagging PCRs are conducted as described above. The Acu1 digestion/inactivation is performed in 20 μl by mixing 0.2 pmol of Acu1-tagged amplicon with 1.25 units Acu1 in 1× Cutsmart buffer. The digestion is incubated at 37° C. for 1 min followed by 1 min at 65° C. The digested reaction is diluted by the addition of 100 μl H₂O and used directly for the ligation. The adaptor ligation is conducted in 10 μl by mixing 2 μl of ligase buffer, 0.5 μl T4 ligase (Invitrogen), 0.5 μl of the selected adaptor, and 0.5 μl diluted digestion. The reaction is incubated for 10 min at 25° C. The reaction is stopped by incubating 10 min at 65° C. Finally, analytical or quantitative PCR is performed as detailed above.

### One-Pot DTECT2.0

The one-pot DTECT2.0 protocol merges DNA ligation and Acu1 digestion in a single tube. It utilizes an optimized quantity of Acu1-tagged amplicon compatible with the one-pot digestion-ligation reaction. The Acu1-tagging PCRs are conducted as described above. The reactions are conducted in a single tube but separated in two independent steps as follows: 0.005 pmol of Acu1 tagged amplicon is digested in a 7 μl reaction by mixing 1 μl Cutsmart buffer, 1.25 μl of diluted Acu1 (Acu1 was diluted 1/10^{th} in 1× Cutsmart buffer) and completed with H₂O. The digestion is incubated for 1 min at 37° C. and 1 min at 65° C. in a thermocycler. Then, 2 μl ligase buffer, 0.5 μl of selected adaptor and 0.5 μl T4 ligase (Invitrogen) are added to the reaction and incubated for 10 min at 25° C. The ligation was stopped by incubation at 65° C. for 10 min. Finally, analytical or quantitative PCR is performed as detailed above.

### Quantification and Statistical Analysis

A standard curve to determine the efficiency of the qPCR amplification and the linearity of the amplification was generated with a plasmid that contains a DTECT ligation product (Addgene #139333) using primers OB18 and OB19 (sequences in Table 1). The linearity of the standard curve has the mathematical formula: y=−3.3245×+7.5504.

Each sample analyzed by qPCR is tested in technical duplicates, and the mean Ct for each sample is calculated. The capture score is defined as the concentration of the captured material for each sample multiplied by 10\hat{ }6. It is measured as follow: Capture score=(10\hat{ }[(Mean Ct−7.5504)/−3.3245])×10\hat{ }6. The reported capture score corresponds to the mean of two independent experiments and is shown as LOG10.

The efficiency score corresponds to the absolute value of the difference in Ct between the specific and non-specific adaptors. It was calculated using the formula: “=ABS(Ct specific adaptor−Ct non-specific adaptor)”. Thus, the reported efficiency score corresponds to the mean of two independent experiments.

TABLE 1
Oligo sequences used in this study

| Name | Oligo description | Sequence SEQ ID | NO: |
|---|---|---|---|
| OB1 | Reverse adaptor | CTGGGGCACGGGTAAGAAGCATTCTGTCTCTCTTCTAAgaattcgagctcggtacccg | 1 |
| OB2 | AA-DTECT Adaptor | cgggtaccgagctcgaattcTTAGAAGAGAGACAGAATGCTTCTTACCCGTGCCCCAGAA | 2 |
| OB3 | AC-DTECT Adaptor | cgggtaccgagctcgaattcTTAGAAGAGAGACAGAATGCTTCTTACCCGTGCCCCAGAC | 3 |
| OB4 | AG-DTECT Adaptor | cgggtaccgagctcgaattcTTAGAAGAGAGACAGAATGCTTCTTACCCGTGCCCCAGAG | 4 |
| OB5 | AT-DTECT Adaptor | cgggtaccgagctcgaattcTTAGAAGAGAGACAGAATGCTTCTTACCCGTGCCCCAGAT | 5 |
| OB6 | CA-DTECT Adaptor | cgggtaccgagctcgaattcTTAGAAGAGAGACAGAATGCTTCTTACCCGTGCCCCAGCA | 6 |
| OB7 | CC-DTECT Adaptor | cgggtaccgagctcgaattcTTAGAAGAGAGACAGAATGCTTCTTACCCGTGCCCCAGCC | 7 |
| OB8 | CG-DTECT Adaptor | cgggtaccgagctcgaattcTTAGAAGAGAGACAGAATGCTTCTTACCCGTGCCCCAGCG | 8 |
| OB9 | CT-DTECT Adaptor | cgggtaccgagctcgaattcTTAGAAGAGAGACAGAATGCTTCTTACCCGTGCCCCAGCT | 9 |
| OB10 | GA-DTECT Adaptor | cgggtaccgagctcgaattcTTAGAAGAGAGACAGAATGCTTCTTACCCGTGCCCCAGGA | 10 |
| OB11 | GC-DTECT Adaptor | cgggtaccgagctcgaattcTTAGAAGAGAGACAGAATGCTTCTTACCCGTGCCCCAGGC | 11 |
| OB12 | GG-DTECT Adaptor | cgggtaccgagctcgaattcTTAGAAGAGAGACAGAATGCTTCTTACCCGTGCCCCAGGG | 12 |
| OB13 | GT-DTECT Adaptor | cgggtaccgagctcgaattcTTAGAAGAGAGACAGAATGCTTCTTACCCGTGCCCCAGGT | 13 |
| OB14 | TA-DTECT Adaptor | cgggtaccgagctcgaattcTTAGAAGAGAGACAGAATGCTTCTTACCCGTGCCCCAGTA | 14 |
| OB15 | TC-DTECT Adaptor | cgggtaccgagctcgaattcTTAGAAGAGAGACAGAATGCTTCTTACCCGTGCCCCAGTC | 15 |
| OB16 | TG-DTECT Adaptor | cgggtaccgagctcgaattcTTAGAAGAGAGACAGAATGCTTCTTACCCGTGCCCCAGTG | 16 |
| OB17 | TT-DTECT Adaptor | cgggtaccgagctcgaattcTTAGAAGAGAGACAGAATGCTTCTTACCCGTGCCCCAGTT | 17 |
| OB18 | PB1072 | Gcaattcctcacgagacccgtcctg | 18 |
| OB19 | PB1073 | Cgggtaccgagctcgaattcttagaag | 19 |
| OB20 | Fwd del 69-70 | TTTTTCTTGTTTTATTGCCACTAGTCT | 20 |
| OB21 | Rev del 69-70 | CAACTTTTGTTGTTTTTGTGGTAATAA | 21 |
| OB22 | AcuI#1 del69-70 | GCAATTCCTCACGAGACCCGTCCTGTTTCCAATGTTACTTCTGAAGGGTTCCATGCTATA | 22 |
| OB23 | AcuI#2 del69-70 | GCAATTCCTCACGAGACCCGTCCTGTTTTCCAATGTTACTCTGAAGTGGTTCCATGCTAT | 23 |
| OB24 | AcuI#3 del69-70 | GCAATTCCTCACGAGACCCGTCCTGTCAAACCTCTTAGTACTGAAGCCATTGGTCCCAGA | 24 |
| OB25 | AcuI#4 del69-70 | GCAATTCCTCACGAGACCCGTCCTGATCAAACCTCTTAGTCTGAAGACCATTGGTCCCAG | 25 |
| OB26 | Fwd RBD | ACTGTGTTGCTGATTATTCTGTCCTAT | 26 |
| OB27 | Rev RBD | GTAATGTCAAGAATCTCAAGTGTCTGT | 27 |
| OB28 | AcuI#1 K417N | GCAATTCCTCACGAGACCCGTCCTGAGACAAATCGCTCCACTGAAGGGGCAAACTGGAAA | 28 |
| | AcuI#2 K417N | | |
| OB29 | AcuI#1 K417T | GCAATTCCTCACGAGACCCGTCCTGCAGACAAATCGCTCCCTGAAGAGGGCAAACTGGAA | 29 |
| OB30 | AcuI#2 K417T | GCAATTCCTCACGAGACCCGTCCTGTCAGACAAATCGCTCCTGAAGCAGGGCAAACTGGA | 30 |
| OB31 | AcuI#3 K417T | GCAATTCCTCACGAGACCCGTCCTGCTGGTAATTTATAATCTGAAGTATAATCAGCAATC | 31 |
| | AcuI#4 K417T | | |
| OB32 | AcuI#3 K417N | GCAATTCCTCACGAGACCCGTCCTGTCTGGTAATTTATAACTGAAGTTATAATCAGCAAT | 32 |
| OB33 | AcuI#4 K417N | GCAATTCCTCACGAGACCCGTCCTGATCTGGTAATTTATACTGAAGATTATAATCAGCAA | 33 |
| OB34 | AcuI#1 S477N | GCAATTCCTCACGAGACCCGTCCTGTATTTCAACTGAAATCTGAAGCTATCAGGCCGGTA | 34 |
| OB35 | AcuI#2 S477N | GCAATTCCTCACGAGACCCGTCCTGATATTTCAACTGAAACTGAAGTCTATCAGGCCGGT | 35 |
| OB36 | AcuI#3 S477N | GCAATTCCTCACGAGACCCGTCCTGTAAAACCTTCAACACCTGAAGCATTACAAGGTGTG | 36 |
| OB37 | AcuI#4 S477N | GCAATTCCTCACGAGACCCGTCCTGTTAAAACCTTCAACACTGAAGCCATTACAAGGTGT | 37 |
| OB38 | AcuI#1 E484K | GCAATTCCTCACGAGACCCGTCCTGAGGCCGGTAGCACACCTGAAGCTTGTAATGGTGTT | 38 |
| OB39 | AcuI#2 E484K | GCAATTCCTCACGAGACCCGTCCTGCAGGCCGGTAGCACACTGAAGCCTTGTAATGGTGT | 39 |
| OB40 | AcuI#3 E484K | GCAATTCCTCACGAGACCCGTCCTGTTGTAAAGGAAAGTACTGAAGACAATTAAAACCTT | 40 |
| OB41 | AcuI#4 E484K | GCAATTCCTCACGAGACCCGTCCTGATTGTAAAGGAAAGTCTGAAGAACAATTAAAACCT | 41 |
| OB42 | AcuI#1 Q498R | GCAATTCCTCACGAGACCCGTCCTGTTACTTTCCTTTACACTGAAGATCATATGGTTTCC | 42 |
| OB43 | AcuI#2 Q498R | GCAATTCCTCACGAGACCCGTCCTGGTTACTTTCCTTTACCTGAAGAATCATATGGTTTC | 43 |
| OB44 | AcuI#3 Q498R | GCAATTCCTCACGAGACCCGTCCTGATGGTTGGTAACCAACTGAAGCACCATTAGTGGGT | 44 |
| OB45 | AcuI#4 Q498R | GCAATTCCTCACGAGACCCGTCCTGTATGGTTGGTAACCACTGAAGACACCATTAGTGGG | 45 |
| OB46 | AcuI#1 N501Y | GCAATTCCTCACGAGACCCGTCCTGCTTTACAATCATATGCTGAAGGTTTCCAACCCACT | 46 |
| OB47 | AcuI#2 N501Y | GCAATTCCTCACGAGACCCGTCCTGCCTTTACAATCATATCTGAAGGGTTTCCAACCCAC | 47 |
| OB48 | AcuI#3 N501Y | GCAATTCCTCACGAGACCCGTCCTGTACTCTGTATGGTTGCTGAAGGTAACCAACACCAT | 48 |
| OB49 | AcuI#4 N501Y | GCAATTCCTCACGAGACCCGTCCTGCTACTCTGTATGGTTCTGAAGGGTAACCAACACCA | 49 |

### DNA Fragments:

SARS-CoV-2 WT (encodes 2 regions of the reference S protein):
(SEQ ID NO: 50)
ATCAAGCTTTTTTTCTTGTTTTATTGCCACTAGTCTCTAGTCAGTGTGT
TAATCTTACAACCAGAACTCAATTACCCCCTGCATACACTAATTCTTTCACACGTGGT
GTTTATTACCCTGACAAAGTTTTCAGATCCTCAGTTTTACATTCAACTCAGGACTTGTT
CTTACCTTTCTTTTCCAATGTTACTTGGTTCCATGCTATACATGTCTCTGGGACCAAT
GGTACTAAGAGGTTTGATAACCCTGTCCTACCATTTAATGATGGTGTTTATTTTGCTT
CCACTGAGAAGTCTAACATAATAAGAGGCTGGATTTTTGGTACTACTTTAGATTCGAA
GACCCAGTCCCTACTTATTGTTAATAACGCTACTAATGTTGTTATTAAAGTCTGTGAA
TTTCAATTTTGTAATGATCCATTTTTGGGTGTTTATTACCACAAAAACAACAAAAGTTG
ACTGTGTTGCTGATTATTCTGTCCTATATAATTCCGCATCATTTTCCACTTTTAAGTGT
TATGGAGTGTCTCCTACTAAATTAAATGATCTCTGCTTTACTAATGTCTATGCAGATTC
ATTTGTAATTAGAGGTGATGAAGTCAGACAAATCGCTCCAGGGCAAACTGGAAAGAT
TGCTGATTATAATTATAAATTACCAGATGATTTTACAGGCTGCGTTATAGCTTGGAATT
CTAACAATCTTGATTCTAAGGTTGGTGGTAATTATAATTACCTGTATAGATTGTTTAGG
AAGTCTAATCTCAAACCTTTTGAGAGAGATATTTCAACTGAAATCTATCAGGCCGGTA
GCACACCTTGTAATGGTGTTGAAGGTTTTAATTGTTACTTTCCTTTACAATCATATGGT
TTCCAACCCACTAATGGTGTTGGTTACCAACCATACAGAGTAGTAGTACTTTCTTTTG
AACTTCTACATGCACCAGCAACTGTTTGTGGACCTAAAAAGTCTACTAATTTGGTTAA
AAACAAATGTGTCAATTTCAACTTCAATGGTTTAACAGGCACAGGTGTTCTTACTGAG
TCTAACAAAAAGTTTCTGCCTTTCCAACAATTTGGCAGAGACATTGCTGACACTACTG
ATGCTGTCCGTGATCCACAGACACTTGAGATTCTTGACATTACGGATCCATC
SARS-CoV-2 del69-70 K417T S477N Q498R (encodes 2 regions of the S
protein with 4 variants):
(SEQ ID NO: 51)
ATCAAGCTTTTTTTCTTGTTTTATTGCCACTAGTCTCTAGTCAGTGTGT
TAATCTTACAACCAGAACTCAATTACCCCCTGCATACACTAATTCTTTCACACGTGGT
GTTTATTACCCTGACAAAGTTTTCAGATCCTCAGTTTTACATTCAACTCAGGACTTGTT
CTTACCTTTCTTTTCCAATGTTACTTGGTTCCATGCTATATCTGGGACCAATGGTACT
AAGAGGTTTGATAACCCTGTCCTACCATTTAATGATGGTGTTTATTTTGCTTCCACTG
AGAAGTCTAACATAATAAGAGGCTGGATTTTTGGTACTACTTTAGATTCGAAGACCCA
GTCCCTACTTATTGTTAATAACGCTACTAATGTTGTTATTAAAGTCTGTGAATTTCAAT
TTTGTAATGATCCATTTTTGGGTGTTTATTACCACAAAAACAACAAAAGTTGACTGTGT
TGCTGATTATTCTGTCCTATATAATTCCGCATCATTTTCCACTTTTAAGTGTTATGGAG
TGTCTCCTACTAAATTAAATGATCTCTGCTTTACTAATGTCTATGCAGATTCATTTGTA
ATTAGAGGTGATGAAGTCAGACAAATCGCTCCAGGGCAAACTGGAACGATTGCTGAT
TATAATTATAAATTACCAGATGATTTTACAGGCTGCGTTATAGCTTGGAATTCTAACAA
TCTTGATTCTAAGGTTGGTGGTAATTATAATTACCTGTATAGATTGTTTAGGAAGTCTA
ATCTCAAACCTTTTGAGAGAGATATTTCAACTGAAATCTATCAGGCCGGTAACACACC
TTGTAATGGTGTTGAAGGTTTTAATTGTTACTTTCCTTTACAATCATATGGTTTCCGAC
CCACTAATGGTGTTGGTTACCAACCATACAGAGTAGTAGTACTTTCTTTTGAACTTCT
ACATGCACCAGCAACTGTTTGTGGACCTAAAAAGTCTACTAATTTGGTTAAAAACAAA
TGTGTCAATTTCAACTTCAATGGTTTAACAGGCACAGGTGTTCTTACTGAGTCTAACA
AAAAGTTTCTGCCTTTCCAACAATTTGGCAGAGACATTGCTGACACTACTGATGCTGT
CCGTGATCCACAGACACTTGAGATTCTTGACATTACGGATCCATC
SARS-CoV-2 K417N, E484K and N501Y(encodes 2 regions of the S
protein with 3 variants):
(SEQ ID NO: 52)
ATCAAGCTTACTGTGTTGCTGATTATTCTGTCCTATATAATTCCGCATC
ATTTTCCACTTTTAAGTGTTATGGAGTGTCTCCTACTAAATTAAATGATCTCTGCTTTA
CTAATGTCTATGCAGATTCATTTGTAATTAGAGGTGATGAAGTCAGACAAATCGCTCC
AGGGCAAACTGGAAATATTGCTGATTATAATTATAAATTACCAGATGATTTTACAGGC
TGCGTTATAGCTTGGAATTCTAACAATCTTGATTCTAAGGTTGGTGGTAATTATAATTA
CCTGTATAGATTGTTTAGGAAGTCTAATCTCAAACCTTTTGAGAGAGATATTTCAACT
GAAATCTATCAGGCCGGTAGCACACCTTGTAATGGTGTTAAAGGTTTTAATTGTTACT
TTCCTTTACAATCATATGGTTTCCAACCCACTTATGGTGTTGGTTACCAACCATACAG
AGTAGTAGTACTTTCTTTTGAACTTCTACATGCACCAGCAACTGTTTGTGGACCTAAA
AAGTCTACTAATTTGGTTAAAAACAAATGTGTCAATTTCAACTTCAATGGTTTAACAGG
CACAGGTGTTCTTACTGAGTCTAACAAAAAGTTTCTGCCTTTCCAACAATTTGGCAGA
GACATTGCTGACACTACTGATGCTGTCCGTGATCCACAGACACTTGAGATTCTTGAC
ATTACGGATCCATC

### REFERENCES

1 Wu, F. et al. A new coronavirus associated with human respiratory disease in China. Nature 579, 265-269, doi: 10.1038/s41586-020-2008-3 (2020).
2 Zhou, P. et al. A pneumonia outbreak associated with a new coronavirus of probable bat origin. Nature 579, 270-273, doi: 10.1038/s41586-020-2012-7 (2020).
3 Zhu, N. et al. A Novel Coronavirus from Patients with Pneumonia in China, 2019. N Engl J Med 382, 727-733, doi: 10.1056/NEJMoa2001017 (2020).
4 Sudre, C. H. et al. Attributes and predictors of long COVID. Nat Med, doi:10.1038/s41591-021-01292-y (2021).
5 Grubaugh, N. D., Hodcroft, E. B., Fauver, J. R., Phelan, A. L. & Cevik, M. Public health actions to control new SARS-CoV-2 variants. Cell 184, 1127-1132, doi:10.1016/j.cell.2021.01.044 (2021).
6 https://covid19.who.int.
7 Rambaut, A. et al. A dynamic nomenclature proposal for SARS-CoV-2 lineages to assist genomic epidemiology. Nat Microbiol 5, 1403-1407, doi:10.1038/s41564-020-0770-5 (2020).
8 Tegally H., W. E., Giovanetti M., Iranzadeh A., Fonseca V., Giandhari J., Doolabh D., Pillay S., James San E., Msomi N., Mlisana K., von Gottberg A., Walaza S., Allam M., Ismail A., Mohale T., Glass A., Engelbrecht S., Van Zyl G., Preiser W., Petruccione F., Sigal A., Hardie D., Marais G., Hsiao M., Korsman S., Davies M-A., Tyers L., Mudau I., York D., Maslo C., Goedhals D., Abrahams S., Laguda-Akingba O., Alisoltani-Dehkordi A., Godzik A., Kurt Wibmer C., Trevor Sewell B., Lourenço J., Carlos Junior Alcantara L., Kosakovsky Pond S., Weaver S., Martin D., Lessells D., Bhiman J., Williamson C., de Oliveira T. Emergence and rapid spread of a new severe acute respiratory syndrome-related coronavirus 2 (SARS-CoV-2) lineage with multiple spike mutations in South Africa. MedRxiv, doi:https://doi.org/10.1101/2020.12.21.20248640 (2020).
9 Hoffmann M., A. P., GroßR., Kleger A., Munch J., Pohlmann S. SARS-CoV-2 variants B.1.351 and P.1 escape from neutralizing antibodies. Cell, doi:https://doi.org/10.1016/j.cell.2021.03.036 (2021).
10 Chen, R. E. et al. Resistance of SARS-CoV-2 variants to neutralization by monoclonal and serum-derived polyclonal antibodies. Nat Med, doi: 10.1038/s41591-021-01294-w (2021).
11 Garcia-Beltran, W. F. et al. Multiple SARS-CoV-2 variants escape neutralization by vaccine-induced humoral immunity. Cell, doi: 10.1016/j.cell.2021.03.013 (2021).
12 Madhi, S. A. et al. Efficacy of the ChAdOx1 nCoV-19 Covid-19 Vaccine against the B.1.351 Variant. N Engl J Med, doi: 10.1056/NEJMoa2102214 (2021).
13 Wang, P. et al. Antibody Resistance of SARS-CoV-2 Variants B.1.351 and B.1.1.7. Nature, doi:10.1038/s41586-021-03398-2 (2021).
14 Tegally, H. et al. Sixteen novel lineages of SARS-CoV-2 in South Africa. Nat Med 27, 440-446, doi: 10.1038/s41591-021-01255-3 (2021).
15 Kemp, S. A. et al. SARS-CoV-2 evolution during treatment of chronic infection. Nature, doi: 10.1038/s41586-021-03291-y (2021).
16 Davies, N. G. et al. Increased mortality in community-tested cases of SARS-CoV-2 lineage B.1.1.7. Nature, doi: 10.1038/s41586-021-03426-1 (2021).
17 Zhou, B. et al. SARS-CoV-2 spike D614G change enhances replication and transmission. Nature, doi: 10.1038/s41586-021-03361-1 (2021).
18 Grint, D. J. et al. Case fatality risk of the SARS-CoV-2 variant of concern B.1.1.7 in England, 16 November to 5 February. Euro Surveill 26, doi:10.2807/1560-7917.ES.2021.26.11.2100256 (2021).
19 Liu, Y. et al. The N501Y spike substitution enhances SARS-CoV-2 transmission. bioRxiv, doi: 10.1101/2021.03.08.434499 (2021).
20 Rambaut A., L. N., Pybus O., Barclay W., Barrett J., Carabelli A., Connor T., Peacock T., Robertson D., Volz D., on behalf of COVID-19 Genomics Consortium UK (CoG-UK). Preliminary genomic characterisation of an emergent SARS-CoV-2 lineage in the UK defined by a novel set of spike mutations, <https://virological.org/t/preliminary-genomic-characterisation-of-an-emergent-sars-cov-2-lineage-in-the-uk-defined-by-a-novel-set-of-spike-mutations/563>(2020).
21 Wibmer, C. K. et al. SARS-CoV-2 501Y.V2 escapes neutralization by South African COVID-19 donor plasma. Nat Med, doi: 10.1038/s41591-021-01285-x (2021).
22 Greaney, A. J. et al. Complete Mapping of Mutations to the SARS-CoV-2 Spike Receptor-Binding Domain that Escape Antibody Recognition. Cell Host Microbe 29, 44-57 e49, doi: 10.1016/j.chom.2020.11.007 (2021).
23 Lu, X. et al. US CDC Real-Time Reverse Transcription PCR Panel for Detection of Severe Acute Respiratory Syndrome Coronavirus 2. Emerg Infect Dis 26, doi:10.3201/eid2608.201246 (2020).
24 Gohl, D. M. et al. A rapid, cost-effective tailed amplicon method for sequencing SARS-CoV-2. BMC Genomics 21, 863, doi: 10.1186/s12864-020-07283-6 (2020).
25 Quick, J. nCoV-2019 sequencing protocol V.1, <https://www.protocols.io/view/ncov-2019-sequencing-protocol-bbmuik6w>(2020).
26 Kovacova V., B. K., Paul E., Radvanszka M., Hajdu R., Čabanová V., Sláviková M., Ličková M., Lukáčiková L., Belák A., Roussier L., Kostičová M., Líšková A., Mad'arová L., Štefkovičová M., Reizigová L., Nováková E., Sabaka P., Koščálová A., Brejová B., Vinař T., Nosek J., Čekan P., Klempa B. A novel, room temperature-stable, multiplexed RT-qPCR assay to distinguish lineage B.1.1.7 from the remaining SARS-CoV-2 lineages. MedRxiv, doi:https://doi.org/10.1101/2021.02.09.21251168 (2021).
27 Vogels, C. B. F. et al. PCR assay to enhance global surveillance for SARS-CoV-2 variants of concern. medRxiv, doi:10.1101/2021.01.28.21250486 (2021).
28 Bal, A. et al. Two-step strategy for the identification of SARS-CoV-2 variant of concern 202012/01 and other variants with spike deletion H₆₉-V70, France, August to December 2020. Euro Surveill 26, doi: 10.2807/1560-7917.ES.2021.26.3.2100008 (2021).
29 Chiara, M. et al. Next generation sequencing of SARS-CoV-2 genomes: challenges, applications and opportunities. Brief Bioinform 22, 616-630, doi: 10.1093/bib/bbaa297 (2021).
30 Bull, R. A. et al. Analytical validity of nanopore sequencing for rapid SARS-CoV-2 genome analysis. Nat Commun 11, 6272, doi: 10.1038/s41467-020-20075-6 (2020).
31 Hodcroft, E. B. et al. Want to track pandemic variants faster? Fix the bioinformatics bottleneck. Nature 591, 30-33, doi: 10.1038/d41586-021-00525-x (2021).
32 Lagerborg, K. A. et al. DNA spike-ins enable confident interpretation of SARS-CoV-2 genomic data from amplicon-based sequencing. bioRxiv, doi:10.1101/2021.03.16.435654 (2021).
33 Billon, P. et al. Detection of Marker-Free Precision Genome Editing and Genetic Variation through the Capture of Genomic Signatures. Cell Rep 30, 3280-3295 e3286, doi: 10.1016/j.celrep.2020.02.068 (2020).
34 Anzalone, A. V. et al. Search-and-replace genome editing without double-strand breaks or donor DNA. Nature 576, 149-157, doi:10.1038/s41586-019-1711-4 (2019).
35 Gaudelli, N. M. et al. Programmable base editing of A*T to G*C in genomic DNA without DNA cleavage. Nature 551, 464-471, doi: 10.1038/nature24644 (2017).
36 Komor, A. C., Kim, Y. B., Packer, M. S., Zuris, J. A. & Liu, D. R. Programmable editing of a target base in genomic DNA without double-stranded DNA cleavage. Nature 533, 420-424, doi: 10.1038/nature17946 (2016).
37 Billon, P. et al. CRISPR-Mediated Base Editing Enables Efficient Disruption of Eukaryotic Genes through Induction of STOP Codons. Mol Cell 67, 1068-1079 e1064, doi:10.1016/j.molcel.2017.08.008 (2017).
38 Shu, Y. & McCauley, J. GISAID: Global initiative on sharing all influenza data—from vision to reality. Euro Surveill 22, doi: 10.2807/1560-7917.ES.2017.22.13.30494 (2017).
39 Tomita, N., Mori, Y., Kanda, H. & Notomi, T. Loop-mediated isothermal amplification (LAMP) of gene sequences and simple visual detection of products. Nat Protoc 3, 877-882, doi: 10.1038/nprot.2008.57 (2008).
40 Tanner, N. A., Zhang, Y. & Evans, T. C., Jr. Visual detection of isothermal nucleic acid amplification using pH-sensitive dyes. Biotechniques 58, 59-68, doi: 10.2144/000114253 (2015).
41 Fischbach, J., Xander, N. C., Frohme, M. & Glokler, J. F. Shining a light on LAMP assays—a comparison of LAMP visualization methods including the novel use of berberine. Biotechniques 58, 189-194, doi: 10.2144/000114275 (2015).
42 Ding, X. et al. Ultrasensitive and visual detection of SARS-CoV-2 using all-in-one dual CRISPR-Cas12a assay. Nat Commun 11, 4711, doi:10.1038/s41467-020-18575-6 (2020).

### Example 2

### Abstract

Sequencing technologies, such as Sanger or Next-Generation Sequencing, are the most common methods to detect genomic sequences and variants of interest. Despite their high accuracy, these technologies remain time-consuming and expensive. There is currently no rapid and cost-efficient method that can be efficiently conducted using all-in-one reactions to detect desired genetic signatures. Here, we establish a platform for rapid and accurate capture of genetic signatures, named Dinucleotide signaTurE CapTure version 3 (DTECTv3). We develop and optimize an all-in-one reaction to rapidly capture desired genetic changes that can be prepared from off-the-shelf reagents and using a set of premade adaptors. We also derive multiple detection modalities with complementary strengths for applications requiring quantitative, qualitative, or visual detection of genetic mutations. We apply DTECTv3 to accurately quantify various mutation types, including transition and transversion mutations, small insertions, and deletions from SARS-CoV-2 variants of concern, cancer mutations, or introduced by cutting-edge CRISPR technologies such as base editing and prime editing. DTECTv3 expedites the accurate detection of genetic signatures for routine laboratory experiments for a fraction of a dollar and enriches the toolkit of the detection methods for CRISPR-based precision genome editing.

### Introduction

Identifying variations in DNA sequences is a routine task in basic research for genetic testing, clinical diagnostic, or forensic purposes. Over the past decades, several technologies utilizing Sanger or Next-Generation Sequencing (NGS) platforms have been developed to facilitate the sequencing of DNA molecules, enabling the determination of DNA sequences and the identification of variants. However, although these technologies are easily accessible, as companies offer genomic platforms and sample processing, they remain time-consuming (several days to weeks) and expensive (from several dollars to thousand dollars) for routine laboratory experiments. Moreover, it requires the processing of samples by third parties, which can cause errors and contaminations during sample manipulation. The past two decades have also witnessed the development of an accelerated number of new techniques using variant-specific primers or probes. However, these techniques are not robust because the efficacy and specificity of the detection are strongly dependent on the sequence and mutation. In addition, these approaches lack specificities as they rely on weak and transient nucleic acid interactions to distinguish between genetic variants, which often differ from the reference by only one nucleotide.

The recent democratization of CRISPR-based precision genome editing technologies, including base editing and prime editing, has increased the need for rapid, sensitive, and accurate methods to detect genetic signatures in routine laboratory settings. These novel genome editing technologies are revolutionizing the modeling and correction of variants in cellular and animal models. In particular, base editing introduces transition mutations, and prime editing inserts all types and combinations of genetic changes (transitions, transversions, deletions, and insertions). Therefore, a detection method that can rapidly and easily identify all types of mutations is highly desirable.

The advent of recombinant DNA technology has revolutionized molecular biology by enabling the assembly of novel DNA sequences. The discovery of DNA ligases and restriction enzymes has emulated this revolution by allowing precise cutting and assembly of DNA fragments with partial overlap sequences. In particular, enzymes from the type IIS (S for “shifted”) family have the unique particularity to cut DNA at a shifted but precise distance from their recognition motif, which allows the cleavage of unknown sequences, enabling the development of a variety of applications. Recent advances in molecular cloning approaches, such as Gibson Assembly, have facilitated the assembly of high complexity DNA fragments. The coordinated action of multiple enzymes in an all-in-one ready-to-use reaction has helped democratize these approaches for the complex manipulation of DNA fragments. However, despite the importance of detecting genetic sequences and associated mutations, there are still no all-in-one assays available for rapid detection of genetic signatures for routine laboratory experiments.

### Results

Here we present DTECTv3, a sequencing-free method that leverages two enzymatic activities to simultaneously expose and capture genetic signatures of interest. We unlock rapid concomitant single-step digestion-ligation of signatures by adding competitor DNA fragments that inhibit Acu1 to prevent the digestion of the ligated adaptors and by enhancing the enzymatic reaction to accommodate the two enzymes. Importantly, DTECTv3 only requires a library of 16 premade and adjustable adaptors to capture all possible types of genetic changes. We illustrate this versatility of the adaptors by unlocking rapid isothermal visual detection of variants, which extends the possibilities of this platform beyond quantitative and qualitative detection. We also show that DTECTv3 enables the rapid detection of emerging SARS-CoV-2 variants of concern and various mutation types, including transition, transversion, small insertions, and deletions, introduced by base editing and prime editing. This platform which utilizes an all-in-one capture reaction with premade adaptors will facilitate and accelerate the routine detection of genetic signatures, including genetic changes introduced by genome editing technologies

In previous studies, we have established a technique to detect genetic signatures, called Dinucleotide signaTurE CapTure (DTECT), that leverages two enzymatic activities: a type IIS restriction endonuclease that is programmed to expose targeted genetic signatures of interest and a DNA ligase that attaches DNA adaptors to enable signature detection (FIG. 6a-c). This approach requires the tagging of signatures of interest (FIG. 6a), signature capture (FIG. 6b) and the detection of ligation products (FIG. 6c) to enable sequencing-free detection of genetic signatures.

The locus of interest is first amplified by PCR using an Acu1-tagging primer that contains a small hairpin to introduce a six-nucleotide motif (5′-CTGAAG-3′) recognized by the Type IIS enzyme Acu1 (FIG. 6a, step 1). Notably, PCR efficiency is not affected by mutations at the dinucleotide of interest because the Acu1-tagging primer is juxtaposed to the dinucleotide signature (in blue). This step generates an Acu1-tagged amplicon digested with Acu1 (FIG. 6b, step 2) for the programmable formation of dinucleotide overhang signatures of the dinucleotide. Indeed, introducing the short Acu1 hairpin programs the formation of dinucleotide signatures of interest. Then, the smaller DNA fragment is isolated (FIG. 6b, step 3) using Solid Phase Reversible Immobilization (SPRI) beads to decrease potential interference between the digested fragments and adaptors during ligation. Next, adaptors complementary (FIG. 6b, step 4 in blue) and non-complementary (FIG. 6b, step 4 in brown) to the dinucleotide signatures are used to capture the isolated DNA fragment by ligation (FIG. 6b, step 4). The use of non-complementary adaptors has a critical role in validating the specificity of the detection, as illustrated throughout the manuscript. The captured material can be detected by quantitative (FIG. 6c, step 5) or qualitative (FIG. 6c, step 5) PCR. A quantitative PCR (qPCR) quantifies the relative abundance of different populations of variants, and analytical PCR rapidly assesses the presence/absence of variants. Because the detection of ligated products relies on a unique couple of detection primers (FIG. 6c, in red), all detections have the same efficiencies and no technical variabilities in the quantification between experiments, making DTECT a robust detection method. Unlike sequencing- or probe-based approaches, which rely on weak interactions between oligonucleotides and templates, DTECT is a ligation-based approach that leads to covalent interaction between signatures and adaptors, providing a robust alternative for the detection of genetic variants.

We previously demonstrated that DTECT could readily identify cancer mutations in the bone marrow of cancer patients and for precision genome editing in cell lines, organoids, and animal tissues. The COVID-19 pandemic has illustrated the need for easy-to-conduct and rapid methods for detecting genetic signatures. Strains of SARS-CoV-2 have emerged (e.g., alpha, beta, gamma, and delta variants) with multiple mutations (e.g., K417N, K417T, E484K, and N501Y), which are unique in the different SARS-CoV-2 lineages. For example, K417N and K417T are specific to the beta and gamma variants. These variants increase transmissibility and partially prevent recognition by vaccine-induced antibodies. Given the high versatility of DTECT in identifying all types of dinucleotide signatures, we tested whether the original DTECT (also referred to as DTECTv1) (FIG. 6) is efficient in capturing SARS-CoV-2 genetic signatures. We designed Acu1-tagging primers specific to the SARS-CoV-2 genome and used the regular DTECT adaptors to capture various SARS-CoV-2 signatures that differ between the SARS-CoV-2 reference sequence-specific variants of concern (FIG. 12a). Using synthetic DNA molecules that encode SARS-CoV-2 mutations, we demonstrated that DTECTv1 efficiently captures signatures of the SARS-CoV-2 reference genome with high sensitivity and specificity by quantitative (FIG. 12b) and qualitative detection (FIG. 12c). Moreover, DTECTv1 identifies each variant signature using signature-specific adaptors (FIG. 12a-c). Furthermore, we detected variants that are common between different strains of SARS-CoV-2 (FIG. 12a-c). These experiments illustrate the performance of DTECTv1 to capture various types of specific variants, including transition and transversion mutations, with high specificity and low background (0.08% n=8) (FIG. 12). Altogether, DTECTv1 identifies SARS-CoV-2 signatures with high efficiency and can distinguish between strains with high specificity.

Although DTECTv1 is robust and rapid to execute (˜4-5 hours), it requires two independent enzymatic reactions (digestion and ligation steps, as shown in FIG. 6b steps 2 and 4) and the processing of the digested fragments by precipitation and beads purification (FIG. 6b steps 3). Given that multistep procedures do not facilitate the execution of experiments, we decided to closely evaluate DTECTv1 for its performance and develop an optimized DTECT assay to expedite the signature capture of critical variants of interest, such as SARS-CoV-2 variants of concerns or cancer mutations.

### Acu1 Digestion and Adaptor Ligation are Critical, but the Beads Isolation Step is Dispensable

DTECT utilizes two sequential enzymatic reactions, a restriction digestion (FIG. 6b, step 2) and a ligation (FIG. 6b, step 4), interspaced by a DNA fragment isolation step (FIG. 6b, step 3) to capture specific signatures. The beads isolation step separates the two DNA fragments generated by the Acu1 digestion based on their length so that the adaptors do not compete with the larger DNA fragment. To evaluate the performance of DTECT, we quantified ligation efficiency (referred hereafter as capture efficiency), which corresponds to the quantity of ligated DNA (FIG. 13a), and the specificity of the ligation (referred hereafter as capture specificity score), which corresponds to the difference in cycle threshold (Ct) between the specific dinucleotide signature capture and the background capture using a non-specific adaptor (FIG. 13b).

To test whether these three independent steps (Acu1 digestion, beads isolation, and adaptor ligation) are essential for DTECT, we conducted DTECTv1 to capture the SARS-CoV-2 E484K variant, but we independently omitted each step/enzyme (i.e., Acu1, beads, or ligase) (FIG. 7a). DTECTv1 leads to a robust capture specificity (FIG. 7b) and efficiency (FIG. 7c) of the genetic signature (specific) compared to the E484 Wuhan reference signature (non-specific capture). The omission of Acu1 or T4 ligase abolishes signature capture to the same extent as the non-specific adaptor (FIG. 7c). These data confirm that the Acu1 activity is critical to exposing the overhang signatures and that ligation of the adaptors is required to achieve signature capture. Surprisingly, omitting the beads isolation step affected neither capture specificity (FIG. 7b) nor efficiency (FIG. 7c). An analysis of the product of ligation by analytical PCR (FIG. 13c) confirmed the expected ligation product. These experiments demonstrate that the beads isolation step is dispensable, facilitating the use of DTECT with the minimal requirement for off-the-shelve enzymes (Acu1 and T4 ligase), and shows that DTECTv1 can be enhanced.

### Development of an Accelerated and Simplified DTECTv2 Assay

These data prompted us to reassess each step of DTECTv1 to develop an optimized capture. First, we tested how parameters influence digestion by Acu1. In the DTECTv1 protocol, Acu1 digests the Acu1-tagged amplicon for 60 minutes at 37° C. as recommended by the enzyme suppliers. We conducted a time-course experiment ranging between 10 seconds and 60 minutes to determine the optimal digestion conditions. Surprisingly, we observed that a 10-second incubation leads to a capture efficiency comparable to a 60 min digestion (FIG. 7d). As a control, a reaction without Acu1 incubated for 60 min led to a background capture (FIG. 2d—in green at t=60 min) comparable to the capture using a non-specific adaptor (FIG. 7d—in red). Notably, 10-second incubation does not affect capture specificity, as indicated by the comparable capture to the 60 min reference used in DTECTv1 (FIG. 2d). These data suggest that the digestion of Acu1-tagged DNA amplicon with Acu1 exposes enough dinucleotide signatures for maximal capture in under 10 seconds.

Notably, the Acu1 activity must not persist during adaptor ligation to avoid the digestion of the ligated product (dinucleotide-adaptors) by Acu1. For this reason, Acu1 is inactivated by incubating the reaction at 65° C. for 20 min, as recommended by the Acu1 suppliers. To test whether Acu1 heat inactivation can be accelerated, we initially heat-inactivated Acu1 from 30 seconds to 20 min and then added the Acu1-tagged amplicon to the reaction so that the level of the captured product determines if Acu1 was efficiently inactivated. We restricted the digestion to 1 min at 37° C. because previous experiments revealed that digestion is already completed after 10 seconds (FIG. 7d). As expected, a control reaction without pre-inactivation of Acu1 led to a robust capture (FIG. 7e and FIG. 13e). Interestingly, pre-incubation of Acu1 for 30 seconds at 65° C. completely abolishes capture, similarly to the 20 min reference (FIG. 7e and FIG. 13e), demonstrating that Acu1 denaturation is completed in under 30 seconds, which is consistent with the kinetics of heat-induced denaturation of proteins.

Next, we tested whether the ligation reaction can be optimized to improve signature capture. We titrated the concentration of DNA ligase and determined the conditions that provide the highest capture efficiency and specificity (FIG. 12f). Finally, we tested how the capture efficiency is affected by the incubation time of the ligation reaction. Surprisingly, we found that a 1- or 10-minute incubation at 25° C. is sufficient to obtain maximal ligation comparable to the 1-hour ligation used in DTECTv1 (FIG. 7f). These results reveal that the ligation step can be reduced to 1 min without affecting ligation efficiency and specificity.

Altogether, these experiments suggest that we can enhance DTECTv1 by removing the beads isolation step and by accelerating Acu1 digestion (from 60 min to 1 min), Acu1 inactivation (from 20 min to 30 s), and adaptor ligation (from 60 min to 1 min). To test whether these changes can be combined and to compare DTECTv1 (3 steps; ˜2 hrs 30 min total capture time) to the accelerated DTECTv2 (2 steps; <5 min capture) (FIG. 7g), we first captured the SARS-CoV-2 E484K variant using DTECTv1 or DTECTv2 and used the SARS-CoV-2 E484 signature as a non-specific control. Although the capture efficiency of DTECTv2 was slightly decreased compared to DTECTv1, we confirmed the high efficiency and specificity capture using both DTECTv1 or DTECTv2 protocols (FIG. 7h). To independently validate these results, we captured the signature of a nonsense mutation highly frequent in cancer patients within the PIK3R1 gene (PIK3R1-STOP) or its reference signature (PIK3R1-WT) using DTECTv1 or DTECTv2. Both the PIK3R1 WT and the STOP signatures were captured efficiently and specifically with their respective adaptors (FIG. 7i), showing that the improvements do not negatively affect DTECT performance and that the changes can be successfully combined. These critical enhancements result in a 92% decrease in the duration of the signature capture, unlocking a rapid (˜10 min) and sensitive signature capture.

### Development of an Isothermal Single-Step One-Pot Capture

Removing the bead isolation step makes it possible to envision that the two enzymatic activities (Acu1 digestion and DNA ligation) could be active into an optimized buffer that can accommodate both activities concomitantly.

To test the compatibility of Acu1 and the DNA ligase to work in a single reaction, we conducted a capture in which the two enzymatic reactions are physically separated in two independent steps/tubes in their respective optimal buffers (FIG. 8a, left). We compared it to a reaction that combines the two enzymes with adaptors and buffers (all-in-one tube) for 10 minutes at 25° C. (FIG. 8a, right). DTECTv2 captured the signatures with high specificity (FIG. 8b, green). However, mixing all in one tube did not lead to capture (FIG. 8b, orange). We hypothesized that the single pot digestion/ligation might be inefficient because Acu1 potentially digests the ligated product, which could disfavor the ligation of the adaptors. To test this, we speculated that increasing the concentration of adaptors could displace the enzymatic reaction in favor of the ligation, even though Acu1 remains active. While DTECTv2 captured with the same efficiency regardless of the concentration of adaptors, we observed that increasing the concentration of adaptors (1000×) restores capture in a single reaction (FIG. 8b, in orange). Notably, an analysis of the product of ligation by sequencing (FIG. 14a) confirmed the expected ligation product, which is composed of the SARS-CoV-2 genomic sequence tagged with the Acu1 hairpin ligated to the specific adaptor.

Next, we systematically evaluated the activity of several DNA ligases to determine the most effective ligase to capture signatures in a single pot. The T4 ligase showed the most robust capture activity among the different ligases, followed by the T3 ligase (FIG. 8c and FIG. 14b), consistent with their preference for cohesive ends. The high performance of the T4 ligase prompted us to test multiple commercial T4 ligases, such as heat resistant (Hr) and highly concentrated T4 ligase (Hc). Each T4 ligase performed well at capturing the dinucleotide signature with high sensitivity (FIG. 8c) and specificity (FIG. 14b). In contrast, T7, 9°N, and Taq ligases did not robustly capture the signature (FIG. 8c and FIG. 14b), as these ligases prefer to ligate nicks of adjacent DNA strands. Notably, the one-pot capture using the regular T4 ligase from two different suppliers lead to efficient capture (FIG. 8c and FIG. 14b), confirming the robustness of the single pot digestion-ligation signature capture.

### Enhancement of the Single-Pot Reaction by Buffer Improvement and Acu1 Inhibition

To enhance the concomitant digestion/ligation, we tested whether the composition of the buffer could increase capture efficiency. We tested how the omission of each component affects the capture efficiency to determine a minimal buffer. We observed that removal of the Acu1 buffer improves capture specificity (FIG. 8d). This effect is likely due to the final concentration of Mg2+ that is present in both the Acu1 and ligase buffers. Finally, we tested whether the addition of known ligation enhancers, such as PEG, DMSO, and tween-20, affects ligation efficiency. The addition of none of these components increased capture efficiency regardless of the concentration used (FIG. 8e and FIG. 14c).

One key characteristic of type IIS enzymes is that they do not cleave their recognition motifs but cut DNA at a shifted distance in the bound DNA. Consequently, type IIS enzymes can remain bound to DNA substrates after digestion, and in the case of the ligation of compatible DNA sequences, Acu1 would digest it, preventing the digestion of the adaptors. We hypothesized that the addition of exogenous DNA fragments that contains an Acu1 motif would limit the capacity of Acu1 to digest newly ligated fragments. To test this, we prepared a double-stranded DNA consisting of an Acu1 motif sequence surrounded by 12 nt to avoid competitor cutting by Acu1. The addition of 1 pmol of the competitor, but not a DNA control that lacks the Acu1 motif, stimulated the capture efficiency (FIG. 8e and FIG. 14d). At a higher concentration of competitors, the capture is inhibited probably because the excess of adaptors prevents the digestion of the Acu1-tagged amplicon.

Altogether, we were able to enforce single pot digestion/ligation despite persisting Acu1 activity by 1) increasing the concentration of adaptors, 2) optimizing buffer conditions, and 3) adding a competitor DNA fragment (FIG. 8f).

Altogether, these data establish a set of key optimizations that lead to the development of version 3 of DTECT (DTECTv3) for the rapid capture of genomic signatures (FIG. 8g). The capture works by incubating the Acu1 tagged amplicon with the all-in-one reaction for 10 minutes at room temperature. In addition, the digestion-ligation reaction is not significantly affected by the temperature (i.e., 16, 25, or 37 degrees) (FIG. 14e), which demonstrates the robustness of the approach.

### Purification of the Acu1-Tagged Amplicon is Dispensable

While the previous experiments have established a rapid single pot capture, the purification of Acu1-tagged amplicon limits the throughput and rapidity of the overall protocol. Given the low amount of Acu1-tagged amplicon needed in DTECTv3, we hypothesized that the purification of Acu1-tagged amplicon might be streamlined. We tested whether direct various purification protocols, including gel purification, column purification, or beads purification, are compatible with the single-step digestion/ligation. As a control, direct dilution ( 1/100^{th}) of the Acu1-tagged amplicon was tested. Surprisingly, we observed that the purification of Acu1-tagged amplicon is dispensable as purification or dilution of the PCR leads to comparable capture (FIG. 8f). This data suggests that the Acu1-tagged amplicon might not require purification.

Quantification with DTECTv3 is Accurate and Specific

Next, we tested whether DTECTv3 is accurate and specific for the quantitative detection of genomic signatures. First, we compared whether the 16 dinucleotides are captured with the same efficiency by their respective complementary adaptors. Strikingly, we observed a highly consistent capture between the 16 possible dinucleotides using DTECTv3 (FIG. 9a). Moreover, the number of A/T and G/C within the dinucleotide did not impact ligation efficiency (FIG. 9b), and there is no difference in the position of C/G and A/T nucleotides at the 5′ or 3′ position of the dinucleotide (FIG. 9c).

Second, we tested the accuracy of DTECT to quantify the relative amount of genetic signatures. We mixed various quantities of WT and variant signatures (ratios: 100:0, 75:25, 50:50, 25:75, and 0:100) and captured the respective signatures using DTECTv3. The relative capture frequency was highly quantitative for detecting a mixture of SARS-CoV-2 variants of concerns and a cancer mutation (FIG. 9d and FIG. 15). The expected capture of all 9 variants and WT led to the expected ratios with 0.1%, 26.4%, 50.3%, 76% and 99.9% for the WT signatures and 0.1%, 24%, 49.7%, 73.6% and 99.9% for the variant signatures where 0, 25, 50 and 75% were expected (FIG. 9e-f).

Altogether these experiments demonstrate that the simplified and accelerated DTECTv3 protocol is highly accurate, does not suffer from technical variabilities, and precisely quantifies the relative frequency of genetic signatures.

### DTECTv3 Enables the Rapid Quantification of Base Editing and Prime Editing in Human Cells

Precision genome editing technologies, such as base editing and prime editing, are revolutionizing genetic studies in cellular and animal models.

We tested DTECTv3 to detect newly generated genetic signatures induced by base editing and prime editing. We transfected HEK293T cells with cytosine base editor (CBE) and guide RNAs (gRNAs) that were designed to introduce a premature STOP codon to inactivate a series of key DNA repair genes (FIG. 10a). We targeted 16 genes involved in the DNA damage response to create knockout cell lines. Three days after transfection, the genomic DNA of the pool of edited cells was harvested. We conducted an Acu1 PCR followed by DTECTv3 with quantitative detection of the ligated product. Strikingly, DTECTv3 readily detected and quantified the newly introduced variant signatures (FIG. 10a). DTECTv3 revealed successful editing of 8 genes, low editing of 6 genes (<5% editing), and one gene remained unedited. The relatively low editing level can be explained by the use of CBE-SpRY, which has a lower activity than CBE. Importantly, this experiment demonstrates that DTECTv3 can distinguish between the various level of editing and determine samples with no editing. In addition, we used adaptors specific to the variant dinucleotide in non-edited control samples and confirmed the high specificity of DTECTv3 (data not shown).

Next, we utilized prime editing to introduce various mutation types, including transition, transversion, precise small deletion, and insertions. Prime editing is the most recent and exciting precision genome editing technology developed. Prime editing can introduce virtually any small genomic changes as desired. We first tested whether DTECTv3 identifies newly created genetic signatures by prime editing. DTECTv3 readily detected genomic changes induced by prime editing, including a three-nucleotide insertion (insCTT) and a small deletion (del1T) at the HEK3 locus (FIG. 10b). Second, we introduced a panel of cancer mutations in the various surfaces of the critical DNA replication and repair PCNA gene using prime editing (FIG. 10c). We edited the different surfaces of the PCNA ring with eight prime editing gRNAs (pegRNAs) that introduce transition, transversion, and a combination of transition and transversion mutations (FIG. 10c). Using DTECTv3, we rapidly quantified the frequency of prime editing in all these loci.

We then derived individual clones harboring cancer mutations successfully introduced into the PCNA gene with prime editing. We used qualitative detection of DTECTv3 to determine if cells were homozygous or heterozygous. DTECTv3 accurately determined the genotype of the cells (FIG. 10d), as confirmed by Sanger sequencing (FIG. 10e). Quantification of the number of each allele by DTECT-qPCR was also consistent with Sanger sequencing (data not shown).

These experiments demonstrate that DTECT streamlines the detection of cells edited with base editing and prime editing. DTECT quantifies the relative amount of WT and edited alleles in cellular pools and clones. These experiments illustrate how DTECT can complement NGS or Sanger sequencing, as a quicker and cost-friendly alternative to determine successful editing and genotyping before more complex analysis of the full spectrum of low-frequency edits by NGS or confirmation of genotyping of the clones of interest by Sanger sequencing.

By accelerating and facilitating the detection of precision genome editing, DTECT can positively impact the generation of precisely edited model systems by facilitating the quantification and genotyping of desired genetic changes in which only a PCR on genomic DNA samples is needed before incubating in an all-in-on reaction for 10 min at room temperature to induce signature capture.

### Development of a Visual Detection Method for DTECTv3

One striking advantage of DTECT is that it uses completely customizable adaptors. We hypothesized that by modifying the adaptor sequences, we could envision additional detection modalities of the ligated product.

Loop-mediated isothermal amplification (LAMP) is a sequence-specific isothermal DNA amplification method that produces a large quantity of DNA. The rapid production of DNA modifies the pH, which induces a change in the color of pH-sensitive dyes that can be visualized under blue/UV light. One important limitation of LAMP is that it requires the identification of specific sets of sequences with particular genomic features (distance between sequences, and G/C contents) in the targeted nucleic acid sequence. The mixture of oligonucleotides complementary to the identified target sequences with the Bst DNA polymerase enables rapid exponential nucleic acid amplification at isothermal temperature. The rapid amplification yields a pyrophosphate ion that changes the color of the reaction if a dye, such as calcein, is added in the reaction. LAMP is a rapid and easy visual method to detect the presence of specific nucleic acid sequences. However, LAMP is not efficient at detecting particular variants within the targeted nucleic acid sequence.

We hypothesized that the DTECT adaptor ligation could trigger the reconstitution of the different LAMP oligonucleotide targets, and therefore, loop amplification would start only if the ligation is efficient, meaning if a signature is present, thereby creating a LAMP approach for the detection of genetic variants. To test this hypothesis, we separated in the LAMP sequences between the 5′ end of the Acu1-tagging oligo and adaptors. Consequently, the LAMP targets would only be reconstituted if the ligation is successful, inducing exponential DNA amplification triggering color change. We included F2 and F3 LAMP sequences derived from the SARS-CoV-2 detection in the 5′ end of the Acu1-tagging oligonucleotide, and the F1, B1, B2, and B3 sequences were included in the adaptors (FIG. 10b). We tested two independent detection primer sets that are commonly used that target the ORF1a and gene N genes of SARS-CoV-2 for rapid visual detection of SARS-CoV-2. To test this approach, we captured the SARS-CoV-2 E484K variant with DTECTv3-LAMP using DTECTv3-LAMP Acu1 tagging primers and adaptors. After 30 min incubation of the captured material in the LAMP reaction, we observed a clear change in the color (FIG. 10b) in the sample captured with the specific adaptor. These results were also confirmed by quantitative detection of color change (FIG. 10c).

Altogether, we adapted the widely used LAMP, which only recognizes the presence of nucleic acids, to specifically detect genetic variants. Importantly, we used validated sequence targets so that DTECT-LAMP detects the presence of specific variants and suppresses the biggest limitation of LAMP, which is the need to identify key sequence targets.

### Discussion

Here, we establish and describe the development of a novel platform for the capture of genetic signatures and derive multiple detection modalities enabling qualitative, quantitative, and visual detection.

DTECTv3 only requires the generation of a PCR product (Acu1-tagged amplicon) that amplifies the locus of interest and “tag” the dinucleotide of interest with the Acu1 motif. This PCR can be generated from any source of DNA or reverse-transcribed RNA and requires little starting material. We note that the generation of a PCR product is also a required initial step for the detection by Sanger sequencing or NGS. The PCR amplicon is then incubated in an all-in-one reaction for 10 minutes room at temperature to expose (i.e., digestion) and capture (i.e., ligation) genetic signatures of interest using a library of adaptors. Finally, the ligated product is detected using three possible detection modalities: qualitative or quantitative PCRs or direct visual detection by loop amplification. Notably, a unique advantage of this platform is that the detection utilizes standard oligonucleotides to detect all genetic variants, mutation types, or genomic loci. This is an important advantage as it limits technical variabilities. Consequently, the use of DTECTv3 is facilitated by the use of common all-in-one master mix reactions for the capture and the detection. These all-in-one reactions contain all the required components to capture, and to detect the ligated product through quantitative PCR, analytical PCR, or DTECT-LAMP. Importantly, all reagents and enzymes necessary to build DTECTv3 are commercially available from multiple suppliers, and the master mixes can be stored for extended periods in a freezer. Altogether, the development of streamlined protocols for the accurate detection of genetic signatures dramatically simplifies the detection of genetic sequences of interest for routine laboratory experiments.

To enable concomitant digestion and ligation in a single reaction, we needed to force the ligation and limit Acu1 activity. We solved this problem on three fronts. First, we optimized the conditions of the reaction to enable simultaneous activities. Second, we forced the ligation by increasing the concentration of adaptors. Third, we added a short DNA fragment containing an Acu1 motif to control Acu1 activity. It is possible that upon DNA cleavage, a structural change is induced in Acu1 to facilitate its release from the DNA while stabilizing its interaction if no cleavage occurred. It remains unclear if this can be generalized to other Type IIS enzymes, as they might have different DNA binding kinetics and interactions.

Alternative detection methods include sequencing technologies, such as next-generation sequencing or Sanger sequencing. However, these approaches are expensive, have a considerable turnaround time of several days (Sanger sequencing) to weeks (NGS) (compared to a few hours for DTECT), and require the involvement of third parties. On the other hand, DTECT is accessible because it only requires off-the-shelf reagents (e.g., T4 ligase and Acu1), available from various suppliers, and minimal equipment (e.g., thermocycler and qPCR). In addition, a critical advantage of DTECT is that it uses a standard library of 16 adaptors to detect each possible dinucleotide signature. Thus, DTECT offers significant advantages over approaches utilizing sequencing technologies to rapidly monitor variants. For instance, DTECT identifies all variant types by capturing targeted signatures with a unique library of adaptors and achieves high specificity and sensitivity detection of molecular signatures through a strong covalent ligation (i.e., capture).

DTECT is a robust molecular diagnostic tool with several significant features that makes it more reliable, specific, and efficient than other rapid diagnostic tests that utilize mutation-specific PCR primers and probes to identify variants. Indeed, these methods have a low specificity conferred by a single nucleotide mismatch to differentiate a variant from the reference (e.g., a 25 nt probe/primer: 1/25 nt→4% specificity target). In contrast, DTECT relies on a dinucleotide capture to differentiate the variant from the reference (½ nt→50% difference in the target), resulting in a strong specificity.

DTECT is highly accurate as it is a ligation-based approach that generates covalent phosphodiester bonds between signatures and adaptors, creating stable ligation products, unlike primers/probes approaches which rely on weak and transient nucleic acid interactions. The production of a stable ligated product allowed the development of multiple modalities to analyze the captured material. Moreover, DTECT provides robust internal controls because, in control samples, the WT but not the variant signatures must be detected. Therefore, the capture of the WT signature acts as a positive control, and the capture of the variant signature provides the background capture. Furthermore, each variant can be detected using four independent signatures (2 flanking Acu1-tagging primers from each DNA strand), providing rigorous validations required to deliver high-confidence results for specific applications. Finally, DTECT is a robust qualitative and quantitative approach with limited technical variabilities because it exploits a unique couple of qPCR oligo pairs to analyze the ligation products (FIG. 6a—Step 5). In contrast, other approaches require a unique design and testing of multiple variant-specific probes and oligos for each variant. The ease of capturing desired nucleic acid signatures with standard adaptors and unique qPCR oligo pairs within a common master mix will prove beneficial for the use of DTECT against any variants of interest, or sample type (if the Acu1-tagging PCR can be produced), without requiring additional optimizations or changes in the DTECT protocol.

One of the most appealing advantages of DTECT for further improvements is the flexibility of the adaptors and 5′-end of the Acu1-tagging oligos. For instance, 5′- and 3′-ends of the adaptors are available to add dyes/quenchers, and the modifications in the DNA sequences do not affect DTECT efficiency. Here, we have developed alternative and independent detection approaches. We have successfully coupled DTECT with LAMP by integrating the LAMP-specific sequences into the adaptors and the 5′ sequence of the Acu1-tagging primers. Therefore, upon ligation of the digested product to the adaptors, the LAMP sequences are reconnected, enabling a loop amplification signal to be visualized in real-time. By coupling our optimized single-step DTECTv3 with LAMP, we visually detect variants of interest, including SARS-CoV-2 variants of concern, in ˜40 min (10 min capture+30 min LAMP). DTECTv3-LAMP is very specific as loop amplification occurs specifically in the conditions in which the ligation has been successful.

### Material and Methods

Synthetic DNA molecules containing portions of the SARS-CoV-2 genome with or without mutations were purchased as gBLOCK DNA fragments (IDT). The DNA fragments were resuspended in TE buffer, cloned into the pCR-Blunt II-TOPO vector (ThermoFisher Scientific), and transformed into DH5a. Successful cloning and SARS-CoV-2 sequence were confirmed by Sanger sequencing.

### Preparation of the Library of Adaptors

A unique library of adaptors is used to capture the 16 possible dinucleotide signatures. The library comprises 16 double-stranded DNA adaptors generated from 17 individual oligonucleotides (sequences available in table 2). It contains one constant oligonucleotide (named OB1), which contains a sequence at the 3′ end (5′-gaattcgagctcggtacccg-3′) (SEQ ID NO: 85) for the detection of the ligated products, and 16 individual oligonucleotides, which are composed of a sequence complementary to the constant oligonucleotide and one of the 16 different dinucleotides at their 3′ end (named OB2-OB17).

For detection by DTECTv3-LAMP, the adaptors are prepared from oligonucleotides containing the complementary sequences of the oligo pool to mediate loop amplification. We used two different oligo pools which are used to either detect SARS-CoV-2 ORF1a or geneN (oligo sequences are available in Table 2, Parts 1 and 2).

TABLE 2
Part 1
Oligonucleotides used for DTECTV3-LAMA

| | | |
|---|---|---|
| OB128 | AcuI LAMP-1a E484K | [symbol] |
| OB129 | F3-1a | [symbol] |
| OB130 | FIP-1a | [symbol] |
| OB131 | B3-1a | [symbol] |
| OB132 | BIP-1a | [symbol] |
| OB133 | LB-1a | [symbol] |
| OB134 | AcuI LAMP-N E484K | [symbol] |
| OB135 | F3-N | [symbol] |
| OB136 | FIP-N | [symbol] |
| OB137 | B3-N | [symbol] |
| OB138 | BIP-N | [symbol] |
| OB139 | LB-N | [symbol] |
| OB140 | DTECT_LAMP_1a_constant | [symbol] |
| OB141 | DTECT_LAMP_1a_TA | [symbol] |
| OB142 | DTECT_LAMP_1a_TG | [symbol] |
| OB143 | DTECT_LAMP_N_constant | [symbol] |
| OB144 | DTECT_LAMP_N_TA | [symbol] |
| OB145 | DTECT LAMP_N_TG | [symbol] |

Competitor sequences

| | | |
|---|---|---|
| OB196 | Competitor AcuI for | [symbol] |
| OB197 | Competitor AcuI rev | [symbol] |
| OB198 | Competitor CTL for | [symbol] |
| OB199 | Competitor CTL rev | [symbol] |

Part 2 (sequences continued from Part 1)

| | |
|---|---|
| GTAAAGGAAAGTACTGAAGACAATTAAAACCTT | 63 |
| | 64 |
| | 65 |
| | 66 |
| | 67 |
| | 68 |
| TAAAGGAAAGTACTGAAGACAATTAAAACCTT | 69 |
| | 70 |
| | 71 |
| | 72 |
| | 73 |
| | 74 |
| [symbol] | 75 |
| [symbol] | 76 |
| [symbol] | 77 |
| [symbol] | 78 |
| [symbol] | 79 |
| [symbol] | 80 |
| | 82 |
| | 83 |
| | 84 |
| | 85 |

[symbol]

indicates data missing or illegible when filed

Each oligonucleotide is resuspended at a concentration of 100 μM in TE (10 mM Tris and 0.5 mM EDTA). The annealing reactions are composed of 2.5 μl of the constant oligonucleotide, 2.5 μl of each unique dinucleotide oligonucleotide, and 1× ligase buffer. The reactions are incubated for 5 min at 95° C. to remove any potential secondary structures, followed by a gradual temperature decrease from 95° C. to 15° C. at a ramp rate of 1° C./s. Then, 100 μl H₂O is added to dilute the adaptors at 5 μM. Adaptors are stored at −20° C. or −80° C.

### Design of Acu1 Tagging Primers and PCR

The Acu1-tagging PCR utilizes a pair of primers named “Acu1-tagging primer” and “reverse primer” also referred to as “reverse Acu1 proimer”. The objective of the Acu1-tagging PCR is to insert an Acu1 motif 14 bp (5′-CTGAAG-3′) upstream from a targeted dinucleotide, introduce a handle that is used for the detection, and amplify the locus of interest. The Acu1-tagging primer is a 60 nt long oligonucleotide that contains an Acu1 motif as a hairpin 14 np from the 3′ end of the primer. In addition, it also contains a non-complementary handle sequence of 25 nt (5′-GCAATTCCTCACGAGACCCGTCCTG-3′) (SEQ ID NO: 55) that is used for the detection. Therefore, the Acu1 tagging primer has the following architecture: 5′-N(15)CTGAAGN(14)-3′ (SEQ ID NO: 56) with “N” corresponding to A, T, G, or C bases complementary to the targeted locus.

The reverse primer is designed using Primer 3 (http://bioinfo.ut.ee/primer3-0.4.0/) with a length of “min=25, Opt=27, Max=30” and a Tm of “min=57.0° C., opt=60.0° C., max=63.0° C.”

For DTECTv3-LAMP Acu1-tagging PCR utilizes a different Acu1-tagged primer with the F3 and F2 sequences which are used to detect the SARS-CoV-2 ORF1a or geneN by LAMP. Acu1-tagging primers are 75 nt long oligonucleotide that contains an Acu1 motif as a hairpin 14 np from the 3′ end of the primer. In addition, it also contains a non-complementary handle sequence 5′-CTGCACCTCATGGTCATGTTATGGTTGAGCTGGTAGCAGA-3′ (SEQ ID NO: 57) for ORF1a detection and 5′-TGGCTACTACCGAAGAGCTACCAGACGAATTCGTGGTGG-3′ (SEQ ID NOL: 58) for geneN detection.

The Acu1-tagging PCR is performed in a 25 μl with 1 unit Q5 polymerase (NEB), 1× Q5 buffer, 1 μM of each primer, 10 ng plasmid template, 0.1 mM dNTP in a thermocycler: 95° C. for 30 s; 40 cycles of 95° C. for 10 s, 58° C. for 10 s, 72° C. for 45 s and a final amplification at 72° C. for 1 min. The PCR reaction is loaded on a 2% agarose gel in TAE buffer, and the amplicon is extracted from the gel and column purified (Zymo Research #D4008). The purified Acu1-tagged amplicon is quantified with the nanodrop 2000 and stored at −20° C.

### DTECTv1 Protocol

The original DTECT protocol has been conducted as detailed previously. Briefly, DTECT relies on the amplification of the genomic locus of interest using an Acu1-tagging primer. The purified Acu1 tagged amplicon is digested by Acu1 in a 20 μl reaction as follows: 0.2 pmol Acu1 tagged amplicon, 1.25 units Acu1 (NEB #0641) in 1× CutSmart buffer. The digestion is incubated at 37° C. for 1 hour, followed by heat inactivation at 65° C. for 20 min. The SPRI bead (Agencourt AMPure XP magnetic beads) step separates the digested fragments by mixing beads at a DNA:beads ratio of 1:1.8. 10 μl of digestion is mixed with 18 μl of beads by pipetting up and down ten times and incubated at room temperature for 5 min. The tube is then placed on a magnetic rack, and the supernatant is recovered and diluted in 40 μl H₂O. Next, the ligation of the adaptors is performed in the following reaction: 6.5 μl H₂O, 2 μl of 5× ligase buffer, 0.5 μl T4 ligase (ThermoFisher Scientific), 0.5 μl adaptor, and 0.5 μl of the purified digested product. The ligation reaction is incubated for 1 hour at 25° C. in a thermocycler. The reaction was then stopped by incubating the reaction at 65° C. for 10 min to denature the ligase. The captured material was detected either using quantitative PCR or analytical PCR.

The original DTECT protocol has been conducted as detailed previously. Briefly, DTECT relies on the amplification of the genomic locus of interest using an Acu1-tagging primer. The purified Acu1 tagged amplicon is digested by Acu1 in a 20 μl reaction as follows: 0.2 pmol Acu1 tagged amplicon, 1.25 units Acu1 (NEB #0641) in 1× CutSmart buffer. The digestion is incubated at 37° C. for 1 hour, followed by heat inactivation at 65° C. for 20 min. The SPRI bead (Agencourt AMPure XP magnetic beads) step separates the digested fragments by mixing beads at a DNA:beads ratio of 1:1.8. 10 μl of digestion is mixed with 18 μl of beads by pipetting up and down ten times and incubated at room temperature for 5 min. The tube is then placed on a magnetic rack, and the supernatant is recovered and diluted in 40 μl H₂O. Next, the ligation of the adaptors is performed in the following reaction: 6.5 μl H₂O, 2 μl of 5× ligase buffer, 0.5 μl T4 ligase (ThermoFisher Scientific), 0.5 μl adaptor, and 0.5 μl of the purified digested product. The ligation reaction is incubated for 1 hour at 25° C. in a thermocycler. The reaction was then stopped by incubating the reaction at 65° C. for 10 min to denature the ligase. The captured material was detected either using quantitative PCR or analytical PCR.

The original DTECT protocol has been conducted as detailed previously. Briefly, DTECT relies on the amplification of the genomic locus of interest using an Acu1-tagging primer. The purified Acu1 tagged amplicon is digested by Acu1 in a 20 μl reaction as follows: 0.2 pmol Acu1 tagged amplicon, 1.25 units Acu1 (NEB #0641) in 1× CutSmart buffer. The digestion is incubated at 37° C. for 1 hour, followed by heat inactivation at 65° C. for 20 min. The SPRI bead (Agencourt AMPure XP magnetic beads) step separates the digested fragments by mixing beads at a DNA:beads ratio of 1:1.8. 10 μl of digestion is mixed with 18 μl of beads by pipetting up and down ten times and incubated at room temperature for 5 min. The tube is then placed on a magnetic rack, and the supernatant is recovered and diluted in 40 μl H₂O. Next, the ligation of the adaptors is performed in the following reaction: 6.5 μl H₂O, 2 μl of 5× ligase buffer, 0.5 μl T4 ligase (ThermoFisher Scientific), 0.5 μl adaptor, and 0.5 μl of the purified digested product. The ligation reaction is incubated for 1 hour at 25° C. in a thermocycler. The reaction was then stopped by incubating the reaction at 65° C. for 10 min to denature the ligase. The captured material was detected either using quantitative PCR or analytical PCR.

### DTECT Optimizations

The experiment without bead isolation was carried out following the DTECTv1 procedure, but the bead step was omitted. Without the beads step, the digestion reaction was diluted by adding 100 μl of H₂O. This dilution was subsequently used in regular ligation. In addition, enzymes have been diluted in their working buffer; for example, Acu1 was diluted in 1× Cutsmart buffer, and T4 ligase was diluted in 1× ligase buffer. All reactions were conducted in independent duplicates. All incubations were conducted in a thermocycler at the indicated temperature and duration for better reproducibility.

### DTECTv2 Protocol

The DTECTv2 protocol relies on DTECTv1 but includes several optimizations. For example, the duration of the digestion/inactivation has been shortened, a dilution in H₂O has replaced the bead isolation step, and the adaptor ligation step has been shortened.

The Acu1-tagging PCRs are conducted as described above. The Acu1 digestion/inactivation is performed in 20 μl by mixing 0.2 pmol of Acu1-tagged amplicon with 1.25 units Acu1 in 1× Cutsmart buffer. The digestion is incubated in a thermocycler at 37° C. for 1 min, followed by 1 min at 65° C. The digested reaction is then diluted by the addition of 100 μl H₂O and used directly for the ligation. The adaptor ligation is conducted in 10 μl by mixing 2 μl of ligase buffer, 0.5 μl T4 ligase, 0.5 μl of the selected adaptor, and 0.5 μl diluted digestion. The reaction is incubated for 10 min at 25° C. The reaction is stopped by incubating 10 min at 65° C. Finally, analytical or quantitative PCR is performed as detailed above.

### Competitor Experiments

The competitor consists of two complementary oligonucleotides, which are annealed to create a double-stranded DNA. The competitor sequences are OB196 5′-AGCCTGTGGTTCCTGAAGATCGCGTCCGAT-3′ (SEQ ID NO: 59) with 5′-CTGAAG-3′ the Acu1 motif, and OB197 5′-ATCGGACGCGATCTTCAGGAACCACAGGCT-3′ (SEQ ID NO: 60) with 5′-CTTCAG-3′ the complementary Acu1 motif. Unlike the Acu1 competitor, the control competitor does not contain an Acu1 motif. The sequences of the two oligonucleotides to make the control competitor are 5′-AGCCTGTGGTTCAAAGTCATCGCGTCCGAT-3′ (SEQ ID NO: 61) and 5′-ATCGGACGCGATGACTTTGAACCACAGGCT-3′ (SEQ ID NO: 62).

To produce the competitors, each oligonucleotide is resuspended at a concentration of 100 μM in TE (10 mM Tris and 0.5 mM EDTA). The annealing reactions are composed of 2.5 μl of each complementary oligonucleotide and 1× ligase buffer. The reactions are incubated for 5 min at 95° C. to remove any potential secondary structures, followed by a gradual temperature decrease from 95° C. to 15° C. at a ramp rate of 1° C./s. Then, the competitor is diluted at 5 μM. Competitors are stored at −20° C.

### Optimizations of the Single-Step DTECTv3 Protocol

The one-pot DTECTv3 protocol merges DNA ligation and Acu1 digestion in a single tube. It utilizes an optimized quantity of Acu1-tagged amplicon compatible with the one-pot digestion-ligation reaction. The Acu1-tagging PCRs are conducted as described above. The reactions are conducted in a single tube but separated in two independent steps as follows: 0.005 pmol of Acu1 tagged amplicon is digested in a 7 μl reaction by mixing 1 μl Cutsmart buffer, 1.25 μl of diluted Acu1 (Acu1 was diluted 1/10th in 1× Cutsmart buffer) and completed with H₂O. The digestion is incubated for 1 min at 37° C. and 1 min at 65° C. in a thermocycler. Then, 2 μl ligase buffer, 0.5 μl of the selected adaptor, and 0.5 μl T4 ligase were added to the reaction and incubated for 10 min at 25° C. The ligation was stopped by incubation at 65° C. for 10 min. Finally, analytical or quantitative PCR is performed as detailed above.

### DTECTv3 Protocol and Master Mixes for All-In-One Capture and Detection.

DTECTv3 only requires an Acu1-tagged amplicon. A 2× DTECTv3 master mix is prepared as follows (recipe to prepare 400 DTECTv3 reactions): 290 μl H₂O, 400 μl 5× ligase buffer, 200 μl competitor (1 pmol/μl), 10 μl Acu1 (10 u/μl) and 100 μl T4 ligase (1 u/μl). The capture is conducted in a 5 μl reaction as follows: 2.5 μl 2× DTECTv3 master mix, 0.25 μl adaptor, and 0.005 pmol Acu1 tagged amplicon. The digestion is incubated in a thermocycler at 25° C. for 1 min, 10 min or 1 hour. The reaction is then stopped by incubating the reaction at 65° C. for 30 s. The captured material is then detected either using quantitative PCR, analytical PCR or DTECT-LAMP.

For detection of the captured product by quantitative PCR, a qPCR master mix is prepared. The recipe to prepare 100 DTECTv3-qPCR reactions (900 μl total) is as follows: 500 μl of 2× SYBR Green master mix, 380 μl H₂O, 10 μl of primer OB1 (100 μM), and 10 μl of primer OB2 (100 μM). 9 μl of qPCR master mix is added in each qPCR well and 1 μl of DTECTv3 is added.

### DTECT-LAMP Protocol

An oligo pool containing LAMP oligos F3, FIP, B3, BIP and LB is prepared. The recipe to prepare 100 μl of oligo pool master mix for LAMP detection is as follows: 20 μl H₂O, 4 μl F3 (100 μM), 32 μl FIP (100 μM), 4 μl B3 (100 μM), 32 μl BIP (100 μM), 8 μl LB (100 μM). Sequences of oligonucleotides are in Table 1.

The LAMP detection reaction is prepared as follows: 5 μl 2× WarmStart colorimetric LAMP (NEB #M1800), 0.4 μl H₂O, 1.6 μl betaine (5 M), 0.5 μl oligo pool, and 1 μl of DTECTv3 capture (diluted 1/1000th in H₂O), added in a WarmStart colorimetric LAMP 2× Master mix (NEB #M1800) in a 10 μl reaction and incubated at 65° C. until the red change turned yellow.

To quantify the LAMP reaction, Spectra Max iD3 was used to measure the absorbance levels at wavelengths 415 and 560 nm by incubating the reaction for 2 hours at 65° C.

### Quantification and Statistical Analysis

A standard curve to determine the efficiency of the qPCR amplification and the linearity of the amplification was generated with a plasmid that contains a DTECT ligation product (Addgene #139333) using primers OB18 and OB19 (sequences in Table 1). The linearity of the standard curve has the mathematical formula: y=−3.3245×+7.5504.

Each sample analyzed by qPCR is tested in technical duplicates, and the mean Ct for each sample is calculated. The capture score is defined as the concentration of the captured material for each sample multiplied by 10\hat{ }6. It is measured as follow: Capture score=(10\hat{ }[(Mean Ct−7.5504)/−3.3245])×10\hat{ }6. The reported capture score corresponds to the mean of two independent experiments and is shown as LOG10.

The efficiency score corresponds to the absolute value of the difference in Ct between the specific and non-specific adaptors. It was calculated using the formula: “=ABS(Ct specific adaptor−Ct non-specific adaptor)”. Thus, the reported efficiency score corresponds to the mean of two independent experiments.

The embodiments described herein are intended to be examples only. Alterations, modifications and variations can be effected to the particular embodiments by those of skill in the art. The scope of the claims should not be limited by the particular embodiments set forth herein, but should be construed in a manner consistent with the specification as a whole.

All publications, patents and patent applications mentioned in this Specification are indicative of the level of skill those skilled in the art to which this invention pertains and are herein incorporated by reference to the same extent as if each individual publication patent, or patent application was specifically and individually indicated to be incorporated by reference.

The invention being thus described, it will be obvious that the same may be varied in many ways. Such variations are not to be regarded as a departure from the spirit and scope of the invention, and all such modification as would be obvious to one skilled in the art are intended to be included within the scope of the following claims.

1-79. (canceled)

80. A method of detecting a dinucleotide sequence in a target polynucleotide containing sample from a subject, the method comprising:
(a) contacting the target polynucleotide containing sample with (i) at least one Acu1 tagging primer, and (ii) a at least one reverse Acu1 primer, under condition to generate an Acu1-tagged amplicon;
(b) contacting the Acu1-tagged amplicon with Acu1, one or more variant adaptors at a concentration of about 250 uM, and a ligase, to generate a reaction mixture,
(c) subjecting the reaction mixture to a reaction time and reaction temperature, to generate a ligation product, and
(d) detecting said ligated product.

81. The method of claim 80, further comprising a competitor DNA, optionally OB196 5′-AGCCTGTGGTTCCTGAAGATCGCGTCCGAT-3′ (SEQ ID NO: 59) or OB197 5′-ATCGGACGCGATCTTCAGGAACCACAGGCT-3′ (SEQ ID NO: 60).

82. A method of detecting a dinucleotide sequence in a target polynucleotide containing sample from a subject, the method comprising:
(a) contacting the target polynucleotide containing sample with (i) at least one Acu1 tagging primer, and (ii) a at least one reverse Acu1 primer, under condition to generate an Acu1-tagged amplicon;
(b) contacting the Acu1-tagged amplicon with Acu1, one or more variant adaptors at a concentration of about 250 uM, a competitor DNA, optionally OB196 5′-AGCCTGTGGTTCCTGAAGATCGCGTCCGAT-3′ (SEQ ID NO: 59) or OB197 5′-ATCGGACGCGATCTTCAGGAACCACAGGCT-3′ (SEQ ID NO: 60), and a ligase, to generate a reaction mixture, and
(c) subjecting the reaction mixture to a reaction time and reaction temperature, to generate a ligation product, and
(d) detecting said ligated product.

83. The method of claim 80, wherein said Acu1 tagging primer comprises an Acu1 motif polynucleotide (5′-CTGAAG-3′) positioned 14 bases from the 3′ end of the Acu1 tagging primer.

84. The method of claim 80, wherein the Acu1 tagging primer comprises a detection handle positioned at the 5′ end of the Acu1 tagging primer.

85. The method of claim 84, wherein the detection handle comprises or consists of the sequence 5′-GCAATTCCTCACGAGACCCGTCCTG-3′ (SEQ ID NO: 53).

86. The method of claim 81, wherein the concentration of the competitor DNA is about 1 pmol.

87. The method of claim 80, wherein the ligase is a T4 ligase or a T3 ligase.

88. The method of claim 87, wherein the T4 ligase is a heat resistant (Hi-T4) T4 ligase, a salt-tolerant (Salt-T4) T4 ligase or, a highly concentrated (T4-HC) T4 ligase.

89. The method of claim 80, wherein said detecting is quantitative, semi-quantitative, analytical, or visual.

90. The method of claim 80, wherein the sample is from a eukaryote, a prokaryote, or a virus.

91. The method of claim 80, wherein the subject is a mammal, a plant, a bacterium, a fungus, a protest, or a virus.

92. The method of claim 80, wherein the sample is isolated from a cell, a cell pellet, a cell extract, a tissue, a biopsy, or biological fluid, obtained from the subject.

93. The method of claim 80, wherein the target polynucleotide is a severe acute respiratory syndrome coronavirus 2 (SARS-CoV-2) polynucleotide sample.

94. The method of claim 80, wherein the target polynucleotide is the PIK3R1 gene, a DNA repair gene, or PCNA.

95. The method of claim 80, wherein the dinucleotide is a mutation, or a reference sequence.

96. The method of claim 95, wherein the mutation is a transition, transversion, insertion, or deletion.

97. The method of claim 80, wherein the sample is from a cancer sample.

98. The method of claim 80, wherein the sample is from a nasal swab, a sample from an oropharyngeal swab, a sputum sample, a lower respiratory tract aspirate, a bronchoalveolar lavage, a nasopharyngeal wash/aspirate or a nasal aspirate.

99. The method of claim 80, wherein the subject is a human.

100. A method of detecting a dinucleotide sequence in a target polynucleotide containing sample from a subject, the method comprising:
(a) contacting the target polynucleotide containing sample with (i) at least one LAMP Acu1 tagging primer, and (ii) a at least one reverse LAMP Acu1 primer, under condition to generate a LAMP Acu1-tagged amplicon;
(b) contacting the LAMP Acu1-tagged amplicon with Acu1, one or more LAMP-specific adaptors, and a ligase, to generate a reaction mixture,
(c) subjecting the reaction mixture to a reaction time and reaction temperature, to generate a LAMP ligation product, and
(d) detecting said LAMP ligated product.

101. The method of claim 100, wherein said LAMP-Acu1 tagging primer comprises an Acu1 motif polynucleotide (5′-CTGAAG-3′) positioned 14 bases from the 3′ end of the Acu1 tagging primer.

102. The method of claim 100, wherein the Acu1 tagging primer comprises a F2 and F3 LAMP sequence at the 5′ end of the LAMP Acu1 tagging primer.

103. The method of claim 100, wherein the ligase is a T4 ligase or a T3 ligase.

104. The method of claim 103, wherein the T4 ligase is a heat resistant (Hi-T4) T4 ligase, a salt-tolerant (Salt-T4) T4 ligase or, a highly concentrated (T4-HC) T4 ligase.

105. The method of claim 100, wherein the reaction temperature is between about 16° C. and about 37° C.

106. The method of claim 100, wherein the reaction temperature is about room temperature.

107. The method of claim 100, wherein the reaction time is between 1 min to 1 hour.

108. The method of claim 100, wherein said detecting is quantitative, semi-quantitative, analytical, or visual.

109. The method of claim 100, wherein the sample is from a eukaryote, a prokaryote, or a virus.

110. The method of claim 100, wherein the subject is a mammal, a plant, a bacterium, a fungus, a protest, or a virus.

111. The method of claim 100, wherein the sample is isolated from a cell, a cell pellet, a cell extract, a tissue, a biopsy, or biological fluid, obtained from the subject.

112. The method of claim 100, wherein the target polynucleotide is a severe acute respiratory syndrome coronavirus 2 (SARS-CoV-2) polynucleotide sample.

113. The method of claim 100, wherein the target polynucleotide is the PIK3R1 gene, a DNA repair gene, or PCNA.

114. The method of claim 100, wherein the dinucleotide is a mutation, or a reference sequence.

115. The method of claim 114, wherein the mutation is a transition, transversion, insertion, or deletion.

116. The method of claim 100, wherein the sample is from a cancer sample.

117. The method of claim 100, wherein the sample is from a nasal swab, a sample from an oropharyngeal swab, a sputum sample, a lower respiratory tract aspirate, a bronchoalveolar lavage, a nasopharyngeal wash/aspirate or a nasal aspirate.

118. The method of claim 100, wherein the subject is a human.

119. (canceled)
